(19) United States
(12) Patent Application Publication
(10) Pub. No.: US 2016/0361340 A1
(43) Pub. Date: Dec. 15, 2016

(54) TREATMENT OF CARDIAC DISEASES WITH MODULATORS OF THE HIPPO PATHWAY

(71) Applicants: INSTITUT PASTEUR, Paris (FR); INSTITUT NATIONAL DE LA SANTE ET DE LA RECHERCHE MEDICALE (INSERM), Paris (FR)

(72) Inventors: Sigolene MEILHAC, Le Kremlin-Bicetre (FR); Chiara RAGNI, Sanguinetto (IT); Jean-Francois LE GARREC, Boulogne-Billancourt (FR); Nicolas DIGUET, Paris (FR)

(73) Assignees: Inetitut Pasteur, Paris (FR); Institut National de la Sante et de la Recherche Medicale (INSERM), Paris (FR)

(21) Appl. No.: 15/117,674

(22) PCT Filed: Feb. 11, 2015
(86) PCT No.: PCT/EP2015/052904

§ 371 Date: Aug. 9, 2016

Related U.S. Application Data
(60) Provisional application No. 61/938,480, filed on Feb. 11, 2014.

(51) Int. Cl. A61K 31/7088; A61K 31/407; G01N 33/68; C12Q 1/68; G01N 33/50; C12N 15/113; A61K 49/00

(52) U.S. Cl. CPC A61K 31/7088; C12N 15/113; A61K 31/407; A61K 49/0008; C12Q 1/6883; G01N 33/5061; G01N 33/5026; G01N 33/5023; G01N 33/6893; C12N 2310/14; C12N 2310/11; C12N 2320/30; C12Q 2600/158; C12Q 2600/156; G01N 2333/705; G01N 2333/4703; G01N 2333/47; G01N 2800/32

(57) ABSTRACT

The present invention provides methods of treating and preventing cardiac hypertrophy and heart failure. Further provided are transgenic animals exhibiting altered expression of the atypical cadherin Fat4 and methods using said transgenic animals, or cells isolated therefrom, for the detection of compounds having therapeutic activity toward cardiac hypertrophy or regeneration. Embodiments of the present invention provide methods and composition for therapeutic intervention in cardiac hypertrophy or heart repair by modulating Fat4 and/or Amotl1 (angiomotin-like1). Treatment may include deleting Yap or administering verteporfm. Embodiments of the present invention define the molecular events linking Fat4 and Amotl1 to cardiac growth, and show that Fat4 is required to restrict cardiomyocyte hypertrophy and cardiomyocyte proliferation and that this is mediated by Amotl1.

### BACKGROUND OF THE INVENTION

Many forms of heart diseases display cardiac hypertrophy. This disease is characterized by an increase in the size of terminally differentiated cardio-myocytes and/or by cardio-myocyte enhanced cell proliferation, ultimately leading to the enlargement of the heart size. Cardiac hypertrophy occurs as a result of intrinsic haemodynamic stress, e.g., as a result of diminished heart function in myocardial infarction, or in response to extrinsic biomechanical stress or as a result of genetic variations^{42,43}.

Although an hypertrophic cardiac response may initially be viewed as a beneficial adaptation to pathological stress due to a cardiovascular disease, in the longer term this response becomes de-compensated and can lead to heart failure at least in part through apoptotic and necrotic cell death. Thus, hypertrophy increases the risk of cardiac morbidity and mortality. More particularly, the presence of cardiac hypertrophy is often associated with increases in the incidence of heart failure, ventricular arrhythmias, death following myocardial infarction, decreased LV (left ventricular) ejection fraction, sudden cardiac death, aortic root dilation and a cerebro-vascular event. Cardiac hypertrophy also carries an increased risk for cardiac events such as angina, myocardial infarction, heart failure, serious ventricular arrhythmias and cardiovascular death.

One of the typical signs of cardiac hypertrophy is an increase in the mass of the left ventricle (LV). This can be secondary to an increase in wall thickness and/or an increase in cavity size. Cardiac hypertrophy as a consequence of hypertension usually occurs with an increase in wall thickness, with or without an increase in cavity size. The normal LV mass in men is 135 g and the mass index often is about 71 g/m². In women, the values are 99 g and 62 g/m², respectively. Left ventricle hypertrophy is usually defined as two standard deviations above normal. The typical echo-cardiographic criteria for left ventricle hypertrophy are ≧134 and 110 g/m² in men and women respectively (see Albergel Am. J. Cardiol. 1995, 75:498). In the clinical practice, the presence of left ventricle hypertrophy is more commonly defined by wall thickness values (obtained e.g. from M-mode or 2D images from the parasternal views). Hypertension associated cardiac hypertrophy may also result in interstitial fibrosis. Both factors contribute to an increase in left ventricular stiffness, resulting in diastolic dysfunction and an elevation in left ventricular end diastolic pressure.

In the last years, many efforts have been made to find compounds for treating these kinds of cardiac diseases. Traditional approaches to suppress cardiac hypertrophy have focused on outside-in signaling, e.g., blocking neurohormones (catecholamines, angiotensin, aldosterone), or calcium triggers (L-type Ca²⁺-channel blockers) or target pathological load (vasodilators and diuretics). However, these treatments vary in effectiveness. For example, antihypertensive agents alone are not an effective treatment. And calcium channel blockers may increase risk in patients with abdominal aortic aneurysm.

Therefore, there remains a need of efficient treatments that would be able to prevent and/or reduce cardiac hypertrophy in adults as well as in newborns or children.

It was well-known that Hippo kinases¹ and Hippo effectors^{2,3} are required to regulate heart growth during development. These molecules can also be manipulated to re-activate cardiomyocyte division in the postnatal heart, thus improving heart repair after injury^{14,15}. However, upstream regulators of the Hippo pathway in mammals remained unknown.

Myocardial infarction (i.e., heart attack) is the irreversible necrosis of heart muscle secondary to prolonged ischemia. Within a matter of hours, it results in the death of a billion of cardiomyocytes (heart muscle cells) in the left ventricle, which are replaced with an avascular fibrotic scar. Although various medical interventions have augmented survival rates after myocardial infarction, the fibrotic myocardium mitigates cardiac contractility, leading to a poor long-term prognosis in these patients (Papizan et al., 2014). Infarcts remain a significant cause of mortality and morbidity, owing to the limited regenerative capacity of the mammalian heart. Damage to cardiac function can be progressive and often leads to congestive heart failure (Addis et al., 2013). The prevalence of heart failure in industrialized nations has reached epidemic proportions and continues to rise. It is the leading cause of death in the industrialized world. Despite significant therapeutic advances, the prognosis for patients who are admitted to the hospital with heart failure remains poor, with a 5-year mortality of about 50%, which is worse than that for patients with breast or colon cancer. In the United States, heart failure affects nearly 6 million persons, kills more than 300 000 people per year, and is directly responsible for more than $40 billion in healthcare expenditures (Sanganalmath et al., 2013). Thus, heart failure is a common, lethal, disabling, and expensive disorder.

Despite significant therapeutic advances, the prognosis of patients with heart failure remains poor, and current therapeutic approaches are palliative in the sense that they do not address the underlying problem of the loss of cardiac tissue.

Cellbased therapies for heart repair have the potential to fundamentally transform the treatment of heart failure by eliminating the underlying cause, not just achieving damage control, with improvement of cardiac function and reduction of infarct size.

They represent a promising alternative to heart transplantation which suffers from a lack of matched donor organs. Early attempts of stem cell-based therapies utilized a number of different cell types, including myoblasts and cells from the bone marrow. Although some of these treatments have shown measurable improvements in cardiac function, the transplanted cells failed to transdifferentiate into cardiac muscle and, in some cases, did not electrically integrate into the heart, leading to arrhythmias (Alexander et al., 2010).

Therefore, production of genuine cardiomyocytes is required for long-term improvement of cardiac function. Potential sources of replacement cells include autologous cardiomyocytes derived from induced pluripotent stem (iPS) cells or direct reprogramming (transdifferentiation) to change one terminally differentiated cell type into induced cardiomyocytes (Lescroart and Meilhac, 2012).

In contrast to the resistance of the adult mammalian heart to regeneration, the neonatal heart displays remarkable regenerative potential. Regeneration of the neonatal mouse heart in response to apical amputation or myocardial infarction seems to occur primarily through proliferation of cardiomyocytes rather than activation of a stem cell population (Porrello et al., 2011). Thus, enhancing cardiomyocyte proliferation by exploiting the young heart's innate ability to regenerate during later stages of adulthood seems particularly attractive as an approach for cardiac repair (Papizan et al., 2014).

Multiple signaling molecules have been shown in mouse models to positively regulate cardiomyocyte proliferation, including neuregulin, fibroblast growth factor, insulin-like growth factor (IGF)1, and periostin. However, these agents also cause cardiomyocyte hypertrophy, which may be deleterious to heart function in the long run. More recently, the Hippo signalling pathway has been shown to exert powerful control of the growth of the myocardium during in utero development (Heallen et al., 2011; Xin et al., 2011; Von Gise et al., 2012). When compared with the other signalling pathways involved in cardiac repair, the Hippo pathway stands out by promoting cardiomyocyte proliferative growth and enhancing myocardial recovery after myocardial infarction without stimulating cardiomyocyte hypertrophy. Modulation of the Hippo pathway in the neonatal heart prolongs the neonatal regenerative window, highlighting the potential for enhancing cardiac regeneration (Heallen et al., 2013; Xin et al., 2013).

In this context, the present inventors identified new effectors of the Hippo pathway that participate, in mammals, in heart growth and/or its restriction. Their role in heart growth has been highlighted for the first time in mammals.

More precisely, the present inventors have shown that:

i) Fat4 mutant myocardium is thicker, with increased cardiomyocyte size and proliferation.

ii) The atypical cadherin Fat4 inhibits the Hippo signaling pathway in cardiomyocytes, thereby reducing their proliferation and hypertrophy, and restricting the growth of the heart. In other words, Fat4 is an inhibitor of the Hippo signaling pathway in cardiomyocytes.

iii) The cardiomyocyte hyperproliferation observed in Fat4 mutant animals is mediated by an up-regulation of the transcriptional activity of Yap1, an effector of the Hippo pathway, which was known to affect cell proliferation, size and survival¹¹. The co-transcription factor Yap1 is thus an activator of the Hippo signaling pathway in mammals, which acts downstream of Fat4.

iv) Yap1 is known to physically interact with Angiomotin-like1 (Amotl1), a member of the Angiomotin family. Amot, another member of the family, can translocate to the nucleus together with Yap1, where the complex modulates transcription²². Amotl1 also interacts physically with Fat4. It is translocated to the nucleus when Fat4 is absent.

Conversely, when Fat4 is present, Amotl1 is impaired from entering the nucleus by sequestration in a Fat4 complex. This sequestration prevents Yap1 mediated tissue growth. Amotl1 is thus an activator of the Hippo signaling pathway, which acts downstream of Fat4.

v) Reducing Yap1 or Amotl1 expression leads to the suppression of Fat4 dependent hyperproliferation and therefore to restricted heart growth.

vi) Conversely, reducing Fat4 expression may facilitate the reactivation of cardiomyocyte proliferation induced by phospho-resistant Yap1¹⁵ or Hippo kinase deficiency¹⁴.

These findings have major therapeutic implications for the repair of the failing human heart.

Thus, in one aspect, the present invention provides methods of treating and preventing cardiac hypertrophy and heart failure. These methods involve either the down-regulation of an activator of the Hippo signalling pathway, namely Yap1 and/or Amotl1, or the up-regulation of an inhibitor of the Hippo signalling pathway, namely Fat4. These treatments may include deleting Yap or administering an inhibitor of Yap1 such as verteporfin.

Further provided are screening methods using transgenic animals exhibiting altered expression of Fat4, or cells isolated therefrom, for the detection of compounds having therapeutic activity toward cardiac hypertrophy or regeneration or of compounds increasing heart growth or cardiomyocyte proliferation.

Other screening methods may involve following the subcellular localisation (nuclear translocation) of Amotl1 as an indication of the activation of cell proliferation.

In another aspect, the present invention provides methods for diagnosing cardiac hypertrophy in a subject in need thereof, comprising the detection of the expression level of Fat4, Yap1 and/or Amotl1 in cardiomyocytes of said subjects.

In a final aspect, the present invention provides methods for stimulating cardiomyocyte proliferation so as to increase the heart size and/or to induce heart growth in a subject in need thereof or to amplify populations of cardiomyocytes, for example derived from stem cells (ES, iPS, etc.) or from patient biopsies.

### DETAILED DESCRIPTION OF THE INVENTION

The present inventors identified the molecular events linking Fat4 and Amotl1 to cardiac growth, and showed that Fat4 is required to restrict cardiomyocyte hypertrophy and cardiomyocyte proliferation, and that this restriction involves two activators of the Hippo signalling pathway, namely Amotl1 and Yap1.

More precisely, they show that Fat4 is required to organise cell junctions and sequester Amotl1, preventing excessive heart growth. In the absence of Fat4, Amotl1 is released and, in a complex with Yap1, translocates to the nucleus, bypassing the Hippo kinases. Resulting variations in gene expression promote proliferation and hypertrophy of cardiomyocytes, leading to excessive growth of the myocardium.

Treating methods, diagnosis methods as well as screening methods can be contemplated in light of these new findings.

The present invention proposes to use Fat4-dependent Hippo pathway modulators in cardiac repair. Fat4-dependent Hippo pathway modulators are for example Amotl1 or Yap1, which have been shown to activate cardiac cell hypertrophy and regeneration, or Fat4 itself, which conversely restricts heart growth (see experimental part below).

More precisely, the results of the present inventors highlight that it is possible to:

i) Prevent or reduce heart growth, heart regeneration, and/or cardiomyocyte proliferation by down-regulating the expression of Fat4 dependent Hippo pathway activators, namely Yap1 or Amotl1, or by up-regulating the expression of Fat4 in cardiomyocytes,

ii) Reactivate cardiomyocyte proliferation or enhance heart size by down-regulating the expression of Fat4 or by up-regulating the expression of Fat4 dependent Hippo pathway activators, namely Yap1 or Amotl1 in cardiomyocytes, or by targeting Amotl1 to the nucleus or by preventing the sequestration of Amotl1 at cell junction or in a complex with Fat4.

Fat4 (or FAT Atypical Cadherin 4 or protocadherin Fat4) is encoded by the Fat4 cDNA of SEQ ID NO:1 in mouse (NM_183221.3), SEQ ID NO:2 in human (NM_001291303.1) and SEQ ID NO:3 in rat (NM_001191705.1). The encoded polypeptide is a member of the protocadherin family, involved in planar cell polarity.

These cDNA encode the Fat4 polypeptide of SEQ ID NO:4 (mouse Fat4, NP_899044.3), SEQ ID NO:5 (human Fat4, NP_001278232.1) and SEQ ID NO:6 (rat Fat4, NP_001178634.1), respectively.

Yap1 (or Yes-associated protein 1, also known as YAP65) is encoded by the Yap1 cDNA of SEQ ID NO:7 in mouse (NM_001171147.1), SEQ ID NO:8 in human (NM_001130145.2) and SEQ ID NO:9 in rat (NM_001034002.2). The Yap1 gene is known to play a role in the development and progression of multiple cancers as a transcriptional regulator of this signaling pathway and may function as a potential target for cancer treatment. It encodes the Yap1 polypeptide of SEQ ID NO:10 (mouse Yap1, NP_001164618.1), SEQ ID NO:11 (human Yap1, NP_001123617.1) and SEQ ID NO:12 (rat Yap1, NP_001029174.2), respectively.

Angiomotin-like protein 1 (Amotl1) is a peripheral membrane protein that is a component of tight junctions (TJs). TJs form an apical junctional structure and act to control paracellular permeability and maintain cell polarity. This protein is related to angiomotin, an angiostatin binding protein that regulates endothelial cell migration and capillary formation (Nishimura M, Kakizaki M, Ono Y, Morimoto K, Takeuchi M, Inoue Y, Imai T, Takai Y (February 2002). “JEAP, a novel component of tight junctions in exocrine cells”. J Biol Chem 277 (7): 5583-7). It is encoded by the Amotl1 cDNA having the SEQ ID NO:13 (NM_001081395.1) in mouse, SEQ ID NO:14 in human (NM_130847.2), and SEQ ID NO:15 (XM_008766026.1) in rat. These cDNAs encode the Amotl1 polypeptide of SEQ ID NO:16 (mouse Amotl1, NP_001074864.1), SEQ ID NO:17 (human Amotl1, NP_570899.1) and SEQ ID NO:18 (rat Amotl1, XP_008764248.1), respectively.

Methods for Preventing Cardiac Hypertrophy

In a first aspect, the present invention therefore relates to a method for preventing and/or treating cardiac hypertrophy by reducing heart growth in a mammal, comprising down-regulating the Fat4-dependent activator of the Hippo pathway Yap1 and/or Amotl1 or up-regulating Fat4 in said mammal.

Cardiomyocyte hyperproliferation induces an increase of the heart size that is usually designated as “cardiac hypertrophy” or “mitogenic cardiomyopathy”. Thus, as used herein, the terms “cardiac hypertrophy” and “mitogenic cardiomyopathy” are equivalent.

In a particular embodiment, said method comprises the step of down-regulating Yap1 expression or transcriptional activity in said mammal, more particularly in the cardiomyocytes of said mammal.

Said down-regulation may be carried out by administering an effective amount of an anti-sense nucleotide inhibiting specifically Yap1 gene expression. Said anti-sense nucleotide is for example a siRNA (or dsRNA), a miRNA, a shRNA, a ddRNAi. Nuclease-based technologies such as Zn-finger nuclease, TALE nuclease or Cas9/Crispr systems can also be used to inhibit gene expression.

More specifically, these anti-sense nucleotides have approximately 15 to 30 nucleotides, 19 to 25 nucleotides, or preferably around 19 nucleotides in length. They are for example complementary (strand 1) and identical (strand 2) to a fragment of SEQ ID NO:7, SEQ ID NO:8 or SEQ ID NO:9.

siRNAs are described for example in WO 02/44 321 (MIT/MAX PLANCK INSTITUTE). This application describes a double strand RNA (or oligonucleotides of same type, chemically synthesized) of which each strand has a length of 19 to 25 nucleotides and is capable of specifically inhibiting the post-transcriptional expression of a target gene via an RNA interference process in order to determine the function of a gene and to modulate this function in a cell or body. Also, WO 00/44895 (BIOPHARMA) concerns a method for inhibiting the expression of a given target gene in a eukaryote cell in vitro, in which a dsRNA formed of two separate single strand RNAs is inserted into the cell, one strand of the dsRNA having a region complementary to the target gene, characterized in that the complementary region has at least 25 successive pairs of nucleotides. Persons skilled in the art may refer to the teaching contained in these documents to prepare the siRNAs of the invention.

MicroRNAs (hereafter referred to as miRNAs) are small non-coding RNA molecule (ca. 22 nucleotides) found in plants and animals, which functions in transcriptional and post-transcriptional regulation of gene expression. miRNAs function via base-pairing with complementary sequences within mRNA molecules, usually resulting in gene silencing via translational repression or target degradation.

The skilled person may also use ddRNAi molecules such as those described generic fashion in application WO 01/70949 (Benitec).

Designing anti-sense nucleotides that are efficient in down-regulating Yap1 expression in the targeted cells is well-known in the art.

Numerous programmes are available for designing siRNAs:
“siSearch Program” at:
   http://sonnhammer.cgb.ki.se/siSearch/siSearch_1.6.html (Improved and automated prediction of effective siRNA”, Chaml A M, Wahlesdelt C and Sonnhammer E L L, Biochemical and Biophysical research Communications, 2004).
“SiDirect” at:
   http://design.mai.jp/sidirect/index.php (Direct: highly effective, target-specific siRNA design software for mammalian RNA interference, Yuki Naito et al, Nucleic Acids Res, Vol. 32, N° Web Server Issue ©Oxford University Press, 2004).
“siRNA Target Finder” by Ambion at the address http://www.ambion.com/techlib/misc/siRNA_tools.html
“siRNA design tool” by Whitehead Institute of Biomedical research at the MIT at the address http://jura.wi.mit.edu/pubint/http://iona.wi.mit.edu/siRNAext/

Examples of anti-Yap1 siRNAs that can be used in the methods of the invention are provided in the enclosed listing sequence, as SEQ ID NO:24, SEQ ID NO:25 and SEQ ID NO:27, that are specific of rat Yap1.

In a particularly preferred embodiment, the present invention relates to an anti-sense nucleotide (e.g., a siRNA) inhibiting specifically the expression of Yap1, for use for preventing and/or treating cardiac hypertrophy by reducing heart growth in a mammal.

In another particularly preferred embodiment, the present invention relates to the use of an anti-sense nucleotide (e.g., a siRNA) inhibiting specifically the expression of Yap1, in the manufacture of a medicament that is useful for preventing and/or treating cardiac hypertrophy by reducing heart growth in a mammal.

By “inhibiting specifically”, it is herein meant compounds having an IC50 on the Yap1 protein expression of less than 1 μM, preferably 100 nM, whereas it has an IC50 on any other protein of more than 5 μM or 10 μM.

Said down-regulation may also be carried out by administering an effective amount of a chemical compound that inhibits Yap1 transcriptional activity. Such compound is for example verteporfin or cardiac glycoside digitonin⁴⁴. Alternatively, down-regulation may also be carried out by administering an effective amount of a chemical compound that inhibits Yap1 expression.

In a particularly preferred embodiment, the present invention relates to verteporfin for use for preventing and/or treating cardiac hypertrophy by reducing heart growth in a mammal.

In another particularly preferred embodiment, the present invention relates to the use of verteporfin, in the manufacture of a medicament that are useful for preventing and/or treating cardiac hypertrophy by reducing heart growth in a mammal.

In another particular embodiment, said method comprises the step of down-regulating Amotl1 expression or biological activity in said mammal, more particularly in the cardiomyocytes of said mammal.

As shown in the experimental part below, Amotl1 biological activity is dependent on its translocation to the nucleus, where it transports the transcription co-factor Yap1 in the absence of Fat4 (in the presence of Fat4, Amotl1 is sequestered at cell junctions in a complex involving Fat4). Thus, dowregulating Amotl1 biological activity may be achieved by favoring the interaction of Amotl1 and Fat4, or of Amotl1 to cell junctions, thereby leading to its sequestration out of the nucleus. It is possible to assess this biological activity directly by detecting the subcellular localisation of Amotl1, e.g., by immunohistochemistry or any conventional means, or indirectly by measuring the expression of Amotl1-dependent genes (e. g. , Aurkb, Ccna2, Birc2, Birc5, Cdkn1b, Lyh6, or Acta1).

Thus, down-regulation of Amotl1 biological activity may be carried out by administering inhibitors (e.g., peptides) of Amotl1-Fat4 interaction or of Amotl1-Yap1 interaction, or any compounds (either chemical or peptides) that would sequester Amotl1 out of the cardiomyocyte nucleus. In this aim, it would be possible to use for example cardiac glycoside digitonin⁴⁴.

Preferably, said down-regulation is carried out by administering an effective amount of an anti-sense nucleotide inhibiting specifically Amotl1 gene expression. Said anti-sense nucleotide is for example a siRNA (or dsRNA), a miRNA, a shRNA, a ddRNAi. Nuclease-based technologies such as Zn-finger nuclease, TALE nuclease or Cas9/Crispr systems can also be used to inhibit gene expression.

Designing anti-sense nucleotides that are efficient in down-regulating Amotl1 expression in the targeted cells is well-known in the art.

These anti-sense nucleotides have preferably 15 to 30 nucleotides, 19 to 25 nucleotides, or more preferably around 19 nucleotides in length. They are for example complementary (strand 1) and identical (strand 2) to a fragment of SEQ ID NO:13, SEQ ID NO:14 or SEQ ID NO:15.

Examples of anti-Amotl1 siRNAs that can be used in the methods of the invention are provided in the enclosed listing sequence, as SEQ ID NO:21 to 23, that are specific of rat Amotl1.

In a particularly preferred embodiment, the present invention relates to an anti-sense nucleotide (e.g., a siRNA) inhibiting specifically the expression of Amotl1, for use for preventing and/or treating cardiac hypertrophy by reducing heart growth in a mammal.

In another particularly preferred embodiment, the present invention relates to a compound inhibiting the nuclear translocation of Amotl1or increasing the sequestration of Amotl1 out of the nucleus, for use for preventing and/or treating cardiac hypertrophy by reducing heart growth in a mammal.

In another particularly preferred embodiment, the present invention relates to the use of an anti-sense nucleotide (e.g., a siRNA) inhibiting specifically the expression of Amotl1, in the manufacture of a medicament that is useful for preventing and/or treating cardiac hypertrophy by reducing heart growth in a mammal.

The results of the inventors show that overexpression of an intracellular domain of Fat4 prevents the nuclear localisation of Yap1 and therefore reduces cardiomyocyte proliferation (FIG. 3).

In another particular embodiment, said method comprises the step of up-regulating Fat4 expression or biological activity in said mammal, more particularly in the cardiomyocytes of said mammal.

As shown in the experimental part below, Fat4 biological activity in cardiomyocytes is based on the sequestration of Amotl1 at cell junctions, i.e., out of the nucleus where Amotl1 may induce transcription of many proliferation genes. Thus, upregulating Fat4 biological activity may be achieved by favouring the interaction of Amotl1 and Fat4, thereby leading to the sequestration of Amotl1 out of the nucleus. It is possible to assess this biological activity directly by detecting the colocalisation of Amotl1 with Fat4, e.g., by immunohistochemistry (or any other conventional means), or indirectly by detecting the subcellular localisation of Amotl1 in cardiomyocytes or by measuring the expression of Amotl1-dependent genes (e.g., Aurkb, Ccna2, Birc2, Birc5, Cdkn1b, Lyh6, or Acta1).

Preferably, said up-regulation is achieved by administering a gene therapy vector encoding the Fat4 polypeptide or a fragment of the Fat4 polypeptide or by administering any compound activating the expression of the Fat4 polypeptide.

This vector is for example a viral vector encoding a fragment of the Fat4 polypeptide.

More precisely, this vector can be an AAV vector (e.g., an AAV9 vector, which has a good affinity for cardiomyocytes) encoding Fat4 or a fragment of the Fat4 polypeptide.

Preferably, said fragment contains the intracellular domain of Fat4.

In a preferred embodiment, said mammal is a human. Preferably, said human suffers from cardiac hypertrophy, as defined above.

In another preferred embodiment, said mammal is embryonic or newborn. If it is newborn, it is more preferably one month or less of age, one week or less of age, or one day or less of age.

In a particular embodiment, the present invention relates to a method for reducing heart growth in a mammal, comprising downregulating Yap1 or upregulating Fat4 in the mammal sufficient to restrict heart growth in the mammal, wherein the mammal is embryonic or newborn.

Methods for Inducing Cardiac Regeneration

The growth of the mammalian heart is critical for its contractile function. During development, cardiomyocyte proliferation underlies most of the growth, whereas increase in cell size (hypertrophy) predominates after birth (Li et al., 1996). Although resident stem cells of cardiomyocytes have been detected in the adult heart (Beltrami et al., 2003; Hsieh et al. 2007), their number and contribution to heart regeneration remains anecdotal. By clonal analysis, it was shown that growth of the embryonic myocardium follows an exponential mode of growth, indicating that symmetrical divisions of myocardial precursors or cardiomyocytes underlie heart growth, and that there is no major pool of cardiac stem cells (Meilhac et al., 2003). 1 week after birth in the mouse and about 10 years in human (Li et al., 1996; Bergmann 2009), cardiomyocytes lose their potential of proliferation. This has been shown to be directly associated with the loss of the regeneration potential of the heart (Porrello et al., 2011). However, adult cardiomyocytes retain some potential of proliferation (Bergmann et al., 2009; Senyo et al., 2013; Villa Del Campo et al., 2014). Thus, enhancing cardiomyocyte proliferation in situ in the more mature heart by exploiting the developmental pathways controlling heart growth seems particularly attractive as an approach for cardiac repair.

In another aspect, the present invention relates to a method to induce heart growth in a mammal, comprising down-regulating Fat4 in said mammal. In particular, said method comprises the down-regulation of Fat4 in the cardiomyocytes of said mammal.

In another particular embodiment, said method comprises the step of down-regulating Fat4 expression or biological activity in said mammal, more particularly in the cardiomyocytes of said mammal.

Downregulating Fat4 biological activity may be achieved by impairing the interaction of Amotl1 and Fat4, thereby leading to the liberation of Amotl1 and its translocation in the nucleus. It is possible to assess this biological activity directly by detecting the colocalisation of Amotl1 with Fat4, e.g., by immunohistochemistry (or any other conventional means), or indirectly by detecting the subcellular localisation of Amotl1 in cardiomyocytes or by measuring the expression of Amotl1-dependent genes (e.g., Aurkb, Ccna2, Birc2, Birc5, Cdkn1b, Lyh6, or Acta1).

Alternatively, down-regulating Fat4 expression can be carried out by administering an effective amount of an anti-sense nucleotide inhibiting specifically Fat4 gene expression. Said anti-sense nucleotide is for example a siRNA (or dsRNA), a miRNA, a shRNA, a ddRNAi. Nuclease-based technologies such as Zn-finger nuclease, TALE nuclease or Cas9/Crispr systems can also be used to inhibit gene expression.

Designing anti-sense nucleotides that are efficient in down-regulating Fat4 expression in the targeted cells is well-known in the art.

These anti-sense nucleotides have preferably 15 to 30 nucleotides, 19 to 25 nucleotides, or more preferably around 19 nucleotides in length. They are for example complementary (strand 1) and identical (strand 2) to a fragment of SEQ ID NO:1, SEQ ID NO:2 or SEQ ID NO:3.

Examples of siRNAs that can be used with this respect are provided in the enclosed listing sequence, as SEQ ID NO:19, SEQ ID NO: 20 and SEQ ID NO:26, that are specific of rat Fat4.

In a particularly preferred embodiment, the present invention relates to an anti-sense nucleotide (e.g., a siRNA) inhibiting specifically the expression of Fat4, for use for inducing heart growth in a mammal or for amplifying a population of cardiomyocytes.

In another particularly preferred embodiment, the present invention relates to the use of an anti-sense nucleotide (e.g., a siRNA) inhibiting specifically the expression of Fat4, in the manufacture of a medicament that is useful for inducing heart growth in a mammal.

In a preferred embodiment, said mammal is a human.

The above-mentioned anti-sense nucleotides can be injected into the cells or tissues by lipofection, transduction or electroporation or viral infection (e.g., by using an AAV9 vector). They can be used to specifically destroy the mRNAs encoding Yap1, Fat4 or Amotl1 thereby entailing the possible therapeutic applications mentioned above.

Methods for Producing High Amounts of Cardiomyocytes In Vitro

Enhancing cardiomyocyte proliferation in vitro by exploiting the developmental pathways controlling cardiomyocyte proliferation is also particularly attractive for producing cardiac tissues that could be grafted in a patient.

In another aspect, the present invention relates to an in vitro method for producing high amounts of cardiomyocytes, said method involving the upregulation of Amotl1 or Yap1 in the nucleus of said cells or the down-regulation of Fat4 in the cytoplasm of said cells.

In a preferred embodiment, said method comprises the following steps:

a) obtaining or generating cardiomyocytes,

b) contacting said cardiomyocytes with a compound down-regulating Fat4, or with a compound upregulating nuclear Amotl1 and/or Yap1,

so as to induce proliferation of said cardiomyocytes.

Upregulation of Amotl1 or of Yap1 can be performed for example by transfecting cardiomyocytes with a vector encoding the Amotl1 or the Yap1 polypeptide. Said vector preferably contains a nuclear localisation signal, so that the encoded polypeptide is forced to translocate to the nucleus of the transfected cells. In a preferred embodiment, said vector is an adenovirus. Adequate vectors are disclosed in the experimental part below (nlsAmotl1).

Downregulation of Fat4 can be performed by any of the above-mentioned means.

The in vitro method of the invention can be carried out on primary cardiomyocyte cells that have been extracted from a cardiac tissue (after a biopsy or cardiac surgery, for example).

In a more preferred embodiment, cardiomyocytes are generated by transforming stem cells (either Embryonic stem cells or iPS cells) into cardiomyocytes by a conventional mean (Goumans M. J. et al, Stem Cell Res. 2007; Laflamme M. A. et al, Nat. Biotechnol. 2007; Van Laake et al, Stem Cell Res. 2007; Blin G. et al, The Journal of Clinical Investigation, 2010; Blin et al, Curr Stem Cell Res Ther 2010; Christine L. et al, Science Translational Medicine, 2010).

Diagnosing Methods

In another aspect, the present invention relates to an in vitro method for diagnosing cardiac hypertrophy in a mammal, comprising analyzing the expression level of Fat4 or Amotl1 or Yap1 or detecting inactivating mutations in the polypeptide sequence of Fat4, Yap1 or Amotl1, in a tissue sample from said mammal.

Preferably, if Fat4 expression level is reduced as compared with a reference value, or if the Fat4 polypeptide contains at least one inactivating mutation, then said mammal is suffering from or will develop cardiac hypertrophy.

Preferably, if Yap1 expression level is enhanced as compared with a reference value, then said mammal is suffering from or will develop cardiac hypertrophy.

Preferably, if Amotl1 expression level is enhanced as compared with a reference value, then said mammal is suffering from or will develop cardiac hypertrophy.

In a preferred embodiment, said tissue sample contains cardiomyocytes.

Detection of reduced Fat4, Yap1 or Amotl1 expression level may be achieved by any conventional means (qPCR, ELISA, Immunohistochemistry, etc.).

The term “reference value”, as used herein, refers to the expression level of the Fat4, Yap1 or Amotl1 gene in a reference sample. A “reference sample”, as used herein, means a sample obtained from subjects, preferably two or more subjects, known not to suffer from cardiac hypertrophy. The suitable reference expression levels of Fat4, Yap1 or Amotl1 can be determined by measuring the expression levels of Fat4, Yap1 or Amotl1 in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference value is preferably based on a large number of samples.

As used herein, “Fat4 inactivating mutations” designate any mutations altering the polypeptide sequence of the Fat4 protein that significantly reduce its biological activity. These mutations can be non-sense mutation or missense mutations, leading to the generation of truncated Fat4 polypeptide to an inactive polypeptide (e.g., a mutation in the binding domain to Amotl1). Some inactivating mutations have been disclosed in Cappello et al, 2013 and in Alders et al.

As used herein, “Yap1 or Amotl1 inactivating mutations” are for example any mutations altering their nuclear localisation (e.g., mutations in the interacting domain with Fat4). More precisely, these mutations may prevent their exit from the nucleus or may induce their translocation in the nucleus.

Preferably, if the Yap1 polypeptide contains a mutation that enhances its nuclear localisation, then said mammal is suffering from or will develop cardiac hypertrophy.

Preferably, if the Amotl1 polypeptide contains a mutation that enhances its nuclear localisation, then said mammal is suffering from or will develop cardiac hypertrophy.

When, according to the method of the invention, a mammal is diagnosed as suffering from cardiac hypertrophy, any appropriate treatment reducing heart growth or heart size can be provided.

Traditional treatments that may be used involve e.g., blocking neurohormones (catecholamines, angiotensin, aldosterone), or calcium triggers (L-type Ca²⁺-channel blockers) or target pathological load (vasodilators and diuretics).

Alternatively, it will be advantageous to treat said mammal by upregulating Fat4 or down-regulating Yap1 and/or Amotl1 , as proposed in the above treating methods of the invention.

Accordingly, said down-regulation can be carried out by administering an effective amount of a siRNA targeting Yap1 (such as those having the sequence SEQ ID NO:24, SEQ ID NO: 25 or SEQ ID NO:27) and/or Amotl1 (such as those having the SEQ ID NO:21 to 23).

Alternatively, Yap1 down-regulation can be carried out by administering an effective amount of verteporfin or of any chemical compound inhibiting Yap1 biological activity.

In a preferred embodiment, said mammal is a human. In another preferred embodiment, said human is suspected of suffering from cardiac hypertrophy (for example, its left ventricle has an abnormal increased size, or an increased thickness or an increased cavity size).

The normal LV mass in men is 135 g and the mass index often is about 71 g/m². In women, the values are 99 g and 62 g/m², respectively. Left ventricle hypertrophy is usually suspected when it presents two standard deviations above normal. The typical echo-cardiographic criteria for suspecting left ventricle hypertrophy are thus ≧134 and 110 g/m² in men and women respectively (see Albergel Am. J. Cardiol. 1995, 75:498).

In another preferred embodiment, said mammal is embryonic or newborn. If it is newborn, it is more preferably one month or less of age, one week or less of age, or one day or less of age.

In another aspect, the present invention relates to a method, comprising analyzing a tissue sample from a mammal for a Fat4 mutation, wherein, if the mutation is present, treating the mammal to prevent or reduce cardiac hypertrophy or heart failure.

In a preferred embodiment, said treatment comprises upregulating Fat4, deleting Yap, or administering an effective amount of verteporfin.

Said mutation is for example the “inactivating mutation” disclosed above.

Screening Methods

In other aspects, the present invention relates to methods, comprising administering compounds to a Fat4 mutant mammal, monitoring cardiac hypertrophy or regeneration in the Fat4 mutant mammal, and selecting a compound demonstrating reduction or prevention of cardiac hypertrophy or regeneration or repair in the Fat4 mouse mutant or amplification of cardiomyocyte populations.

In a preferred embodiment, the Fat4 mutant mammal is a Fat4 mouse mutant.

In another preferred embodiment, said Fat4 mutant mammal is embryonic or newborn. If it is newborn, it has more preferably one month or less of age, one week or less of age, or one day or less of age.

In a preferred embodiment, said monitoring comprises quantifying cell proliferation and/or cell shape.

More precisely, the present invention relates to a screening method for identifying compounds that are useful for preventing and/or treating cardiac hypertrophy, said method comprising the following steps:
a) administering a candidate compound to a transgenic mammal being deficient for the Fat4 gene,
b) monitoring cardiac hypertrophy or regeneration in said transgenic mammal before and after step a), and
c) selecting the candidate compound if it induces the reduction of cardiac hypertrophy in said transgenic mammal.

In a preferred embodiment, said step b) involves the monitoring of the expression of Yap1 dependent genes, such as Aurkb, Ccna2, Birc2, Birc5, Cdkn1b, Lyh6, or Acta1. In this case, it is concluded that the candidate compound leads to the “reduction of hypertrophy” when the expression of Yap1 dependent genes is reduced in its presence (as compared with the expression of the same genes prior to its administration). Conversely, it is concluded that the candidate compound leads to “cardiac growth or regeneration” when the expression of Yap1 dependent genes is enhanced in its presence (as compared with the expression of the same genes prior to its administration).

In another preferred embodiment, said step b) comprises quantifying cardiomyocyte proliferation and/or shape. In this case, reduction of hypertrophy is observed when cardiomyocyte proliferation is decreased or when cardiomyocyte size is reduced in the presence of the tested compound. Conversely, an enhanced cardiomyocyte proliferation or size will be a sign of cardiac growth or regeneration so that the candidate compound will not be useful for preventing and/or treating cardiac hypertrophy.

In a preferred embodiment, the transgenic mammal used in the screening method of the invention is a Knock-out Fat4^{-/-} or Fat4^{flox/flox} mammal. Preferably, said mammal is any mammal with the exception of human. For example, it is a Knock-out Fat4^{-/-} mouse or a Knock-out Fat4^{-/-} rat.

In a preferred embodiment, said transgenic mammal is embryonic or newborn, and is preferably having one month or less of age, one week or less of age, or one day or less of age.

In another embodiment, the screening method of the invention is not carried out on a whole animal but rather on cells extracted therefrom.

In this case, the screening method of the invention comprises the following steps:
a) contacting in vitro a candidate compound to at least one cell which is deficient for the Fat4 gene,
b) monitoring the proliferation and/or the size of said at least one cell before and after step a), and
c) selecting the candidate compound if it is able to reduce the proliferation and/or the size of said at least one cell.

In a preferred embodiment, said at least one cell is a cardiomyocyte.

In another preferred embodiment, said at least one cell is a Fat4^{-/-} or Fat4^{flox/flox} human, mouse or rat cardiomyocyte.

The candidate compound is useful for preventing and/or treating cardiac hypertrophy if the proliferation of said at least one cell is decreased or if its size is reduced in its presence (as compared with in its absence). Conversely, an enhanced proliferation or size will be a sign of cardiac growth or regeneration so that the candidate compound will not be useful for preventing and/or treating cardiac hypertrophy.

Cell proliferation and/or size may be assessed by any conventional means, such as microscopy analysis, cell counting, labeling of proliferation markers by immunohistochemistry or flow cytometry etc. or monitoring the expression of cell cycle genes.

In another embodiment, the screening method of the invention involves the monitoring of the expression level of the modulators of the Hippo pathway (Yap1, Amotl1 and/or Fat4) in cardiomyocyte cells. As a matter of fact, if, in cardiomyocytes contacted with a candidate compound, Fat4 expression is enhanced, or if Yap1 or Amotl1 expression is reduced, then said candidate compound is useful for preventing and/or treating cardiac hypertrophy.

One could also look for compounds which will enhance or suppress the proliferative effect of nlsAmotl1 (adenovirus cloned with a nuclear Amotl1).

In a preferred embodiment, the screening method of the invention therefore comprises the following steps:
a) contacting a candidate compound with at least one cardiomyocyte cell,
b) monitoring the expression level of Fat4 in said cell before and after step a), and
c) selecting the candidate compound if contacting said cell with said candidate compound enhances Fat4 expression.

Alternatively, the screening method of the invention may comprise the following steps:
a) contacting a candidate compound with at least one cardiomyocyte cell,
b) monitoring the expression level of Yap1 and/or Amotl1 or the Yap1 and/or Amotl1 subcellular localisation, in said cell before and after step a), and
c) selecting the candidate compound if contacting said cell with said candidate compound reduces Yap1 and/or Amotl1 expression or increase the sequestration of Amotl1 or Yap1 out of the nucleus.

The expression level of Fat4, Amotl1 and/or Yap1 or the subcellular localisation of these polypeptides may be assessed by any conventional means (e.g., by RT-qPCR, ELISA, Immunohistochemistry, etc.).

In another aspect, the present invention relates to a screening method for identifying compounds that are useful for increasing heart size or inducing heart regeneration or for amplifying cardiomyocyte populations, said method comprising the following steps:
a) contacting a candidate compound with at least one cardiomyocyte cell,
b) monitoring the expression level of Fat4 in said cell before and after step a), and
c) selecting the candidate compound if contacting said cell with said candidate compound reduces Fat4 expression.

Alternatively, this screening method requires the following steps:
a) contacting a candidate compound with at least one cardiomyocyte cell,
b) monitoring the expression level of Yap1 and/or Amotl1 in said cell before and after step a), or monitoring their subcellular localisation, and
c) selecting the candidate compound if contacting said cell with said candidate compound enhances Yap1 and/or Amotl1 expression or increases their nuclear translocation or reduces their sequestration out of the nucleus.

The expression level of Fat4, Amotl1 and/or Yap1 or the subcellular localisation of these polypeptides may be assessed by any conventional means (e.g., by RT-qPCR, ELISA, Immunohistochemistry, etc.). The subcellular translocation may be assessed by any conventional means (e.g., Immunohistochemistry, Imagestream, etc).

Kits and Uses Thereof

The present invention finally relates to kits comprising the means to detect the expression level of Fat4, Yap1 and/or Amotl1 in cells or the subcellular localisation of Yap1 and/or Amotl1.

These means can be primers or probes for the specific detection of the presence or absence of the mRNA of these markers.

These kits may also contain a heat-resistant polymerase for PCR amplification, one or more solutions for amplification and/or the hybridisation step, and any reagent with which to detect the said markers, preferably in cardiomyocytes.

According to an embodiment, these kits may alternatively or additionally contain antibodies that are specific of the Fat4, Yap1 and/or Amotl1 proteins.

In this case, the kits of the invention may also contain any reagent adapted for hybridisation or immunological reaction on a solid carrier.

These kits may be used in the screening and/or the diagnosing methods of the invention.

More precisely, they may be used for diagnosing cardiac hypertrophy in a mammal, or for identifying compounds that are useful for preventing and/or treating cardiac hypertrophy or for increasing heart size or inducing heart regeneration.

### FIGURE LEGENDS

FIG. 1. Excessive thickness of Fat4 mutant hearts. Whole mount views (a) and histological sections (b) of Fat4^{+/-} and Fat4^{-/-} neonatal (P0) hearts. The arrowhead points to the flattened apex and double arrows highlight ventricular wall and septum thickness. (c) Quantification of the thickening in Fat4^{+/+} (n=7), Fat4^{+/-} (n=5) and Fat4^{-/-} (n=6) mutants. Scale bar: 500 μm. RV, right ventricle; LV, left ventricle. In all figures, data are presented as means±standard deviations, normalised to the level of control hearts when appropriate. Statistical significance: * p<0.05, ** p<0.01, *** p<0.001.

FIG. 2. Fat4 restricts cell proliferation and hypertrophy. Immunodetection (a) and quantification (b) of the number of mitotic cardiomyocytes positive for phosphorylated histone H3 (PH3) (arrowheads) in Fat4^{+/+} (n=6), Fat4^{+/-} (n=6) and Fat4^{-/-} (n=5) hearts at P0. (c) Percentage of cardiomyocytes per total number of cells, analysed by ImageStream, in Fat4^{Flox/+} (controls, n=7) and Fat4^{Flox/-}; Mesp1^{Cre/+} (mutants, n=6) hearts at P14. (d) Relative transcript levels of cell cycle (Aurkb, Ccna2, Cdc20), cycle exit (Cdkn1b) or survival (Birc2/5) genes in Fat4^{+/+} (n=5), Fat4^{+/-} (n=5) or Fat4^{-/-} (n=7) hearts at P0. (e) Increased number of proliferating Ki67-positive cardiomyocytes, as counted by flow cytometry from primary cell cultures treated with Fat4 siRNA (n=20) compared to control (ctrl) cultures (n=20). (f) Immunodetection of cardiomyocyte cross-sectional area, with an antibody to Caveolin3. (g) Quantification of this in the interventricular septum indicates cell hypertrophy in Fat4^{-/-} (n=6) compared with Fat4^{+/+} (n=9) hearts at P0. (h) Expression of positive (Nppb and Acta1 (skeletal actin)) and negative (Myh6 (α myosin heavy chain)) hypertrophy markers and of a marker of wall stress (Nppa) quantified by RT-qPCR in Fat4^{+/+} (n=5), Fat4^{+/-} (n=5) or Fat4^{-/-} (n=7) hearts at P0. Scale bars: 10 μm.

FIG. 3. Fat4 modulates Hippo signalling. (a) Relative expression of Yap1 target genes in Fat4^{+/+} (n=5), Fat4^{+/-} (n=5) or Fat4^{-/-} (n=7) hearts at P0. (b) Immunodetection of Yap1 localisation in the heart of the indicated genotype at P0. Nuclei are encircled with dashed white lines. (c) Immunodetection of Yap1 localisation in primary cultures of cardiomyocytes treated with the indicated siRNA. (d) Quantification of the increased percentage of nuclei with a strong Yap1 signal when Fat4 is down-regulated (n=6 cultures) relative to the control (n=6). (e) Immunodetection of Yap1 (nuclear, arrows) localisation in primary cultures of cardiomyocytes transfected with the indicated plasmids. (f) Quantification of its nuclear to cytoplasmic localisation, which is decreased when Fat4 is overexpressed (n=14 cells) relative to control GFP (n=9). (g) The number of proliferating Ki67-positive cardiomyocytes, counted by flow cytometry from primary cell cultures, indicates a genetic rescue of Fat4 siRNA by Yap1 siRNA (n=20 ctrl, 20 Fat4, 8 Yap1 and 8 Fat4+Yap1 siRNA cultures). (h) Quantification of the number of mitotic cardiomyocytes positive for PH3 in Fat4^{Flox/+} (controls, n=5), Fat4^{Flox/-}; Mesp1^{Cre/+} (mutants, n=5) and Fat4^{Flox/-}; Yap1^{Flox/+}; Mesp1^{Cre/+} (rescue, n=4) hearts at P0. (i) Percentage of cardiomyocytes, analysed by ImageStream, in Fat4^{Flox/+} (controls, n=7), Fat4^{Flox/-}; Mesp1^{Cre/+} (mutants, n=6) and Fat4^{Flox/-}; Yap1^{Flox/+}; Mesp1^{Cre/+} (rescue, n=3) hearts at P14. (j) Western blot showing normal Yap1 phosphorylation at the Hippo kinase target site. (k) Corresponding quantification, from the blot shown in Supplementary FIG. 3b, in Fat4^{+/+} (n=4), Fat4^{+/-} (n=6) or Fat4^{-/-} (n=4) hearts at P0. (l) Immunodetection of the localisation of phospho-resistant Yap1 (Yap5SA) in primary cultures of cardiomyocytes treated with the indicated siRNA. (m) Quantification of its nuclear to cytoplasmic localisation, which is increased when Fat4 is down-regulated (n=6 cultures) relative to the control (n=6). Ctrl, control; ns, no significant difference. Scale bars: 5 μm in (b), 20 μm in (c), 10 μm elsewhere.

FIG. 4. Amotl1 mediates Fat4 signalling. (a) Immunodetection of cell junctions (arrowheads), marked by N-cadherin (Cadh2) and Plakophilin2 (Pkp2), in E18.5 hearts. They are disorganised in the absence of Fat4. (b) Transmission electron micrographs of Fat4^{+/+} and Fat4^{-/-} hearts at P0. Gap junctions (arrowhead) are absent in mutant hearts, whereas the electron-dense material of desmosomes (arrows) in the intercalated discs is abnormally spread. (c) Immunodetection of Amotl1 and Yap1 (white arrowheads) at similar positions near the cell membrane, marked by vinculin (Vcl), of cardiomyocytes, marked by α-actinin (Actn2) in P0 control hearts. Asterisks indicate the striation of the sarcomeres. (d) Amotl1 is relocalised to cardiomyocyte nuclei (arrowheads) in Fat4^{-/-} hearts at E18.5. *, non-cardiomyocyte nuclei. (e) Quantification of nuclear to cytoplasmic localisation of Amotl1 in Fat4^{+/+} (n=3), Fat4^{+/-} (n=3) or Fat4^{-/-} (n=3). (f) The number of proliferating Ki67-positive cardiomyocytes, counted by flow cytometry from primary cell cultures, indicates a genetic rescue of Fat4 siRNA by Amotl1 siRNA and lower proliferation with Amotl1 siRNA (n=20 ctrl, 20 Fat4, 16 Amotl1 and 16 Fat4+Amotl1 siRNA cultures). (g) Immunodetection of Yap1 localisation in primary cultures of cardiomyocytes infected with the indicated adenoviruses. The inset shows nuclear co-localisation with Ad-HA-(n1s)₃-Amotl1. (h) Increased number of proliferating Ki67-positive cardiomyocytes, as counted by flow cytometry from primary cell cultures infected with nuclear Amotl1 (Ad-HA-(n1s)₃-Amotl1, n=8), compared to controls (Ad-GFP, n=8). Treatment with Yap1 siRNA (n=8) is inhibitory. (i) Immunoprecipitation (IP) of Amotl1-HA from cells transfected or not with Amotl1-HA or Fat4-ΔECD-Flag, which is depleted for the extracellular domain (ECD). IB, immunoblot with the indicated antibodies. (j) Co-localisation of Amotl1-HA, Fat4-ΔECD-Flag and endogenous Yap1 in primary cultures of transfected cardiac cells. The inset shows an enlargement of the boxed region. Scale bars: 0.2 μm in (b), 5 μm in (c), 10 μm elsewhere. Ctrl, control.

FIG. 5. Model for the role of Fat4 in restricting heart growth. Fat4 is required to organise cell junctions and sequester Amotl1, preventing excessive heart growth. In the absence of Fat4, Amotl1 is released and, in a complex with Yap1, translocates to the nucleus, bypassing the Hippo kinases. Resulting variations in gene expression promote proliferation and hypertrophy of cardiomyocytes, leading to excessive growth of the myocardium. Darker red and yellow indicates high levels of Yap1 and Amotl1; lower levels are indicated by a paler colour.

FIG. 6. Fat4 does not impair the coordination of cell divisions in the embryonic heart. (a) Example of E10.5 cardiomyocytes, with labelled nuclei (blue), membranes (red) and cytoplasmic bridge (green). The dotted line indicates the axis of cell division. Scale bar: 10 μm. (b) Distribution of the angle between the axis of cell division and the local transmural axis. In both Fat4^{+/+} and Fat4^{-/-} hearts, the observed distribution (blue), which is significantly different (Wilcoxon U-test two-tailed, p<0.001, n=335 and 524 respectively) from a random spherical distribution (red), indicates a planar bias of the orientation of cell division. (c) Maps of the regions where the axes of cell divisions were significantly coordinated in the plane of the heart surface. The maps, shown as XY projections, summarize axial data from 4 Fat4^{+/+} and 3 Fat4^{-/-} hearts at E10.5 registered on a single template. Isolines delineate regions with an increasing quality of axial coordination (colour coded). The blue ellipse shows the average orientation of cell division in the most extensive region, with the corresponding elongation value 1-(E₂/E₁). The green bars indicate the local average direction per 100 μm×100 μm box, shifted every 20 μm. Nb, number; LV: left ventricle; RV: right ventricle.

FIG. 7. Proliferation of cardiac cells and RNA interference. (a) Quantification of the number of mitotic non-cardiomyocytes positive for PH3 in Fat4^{+/+} (n=6), Fat4^{+/-} (n=6) and Fat4^{-/-} (n=5) hearts at P0 (see FIG. 2a). (b) Immunodetection in Fat4^{+/+} and Fat4^{-/-} hearts at P0 of cardiomyocytes, marked by cardiac troponin I (Tnni3), undergoing cytokinesis, marked by cytoplasmic bridges (arrowheads) positive for AuroraB kinase (Aurkb) and acetylated tubulin (Ac-tub). (c) Quantification of the number of nuclei per cardiomyocyte in control Fat4^{Flox/+} (n=6) and mutant Fat4^{Flox/-}; Mesp1^{Cre/+} (n=6) hearts at P14. (d) Histological sections of control Fat4^{Flox/-} and mutant Fat4^{Flox/-}; Wt1^{Cre/+} hearts at P0. (e) Quantification of the number of mitotic cardiomyocytes positive for PH3 in Fat4^{Flox/+,} (controls, n=5) and Fat4^{Flox/-}; Wt1^{Cre/+} (mutants, n=5) hearts at P0. (f) Efficient down-regulation of Fat4 (n=6 ctrl, 4 Fat4 siRNA-1, 6 siRNA-2, 6 siRNA-3 cultures), Yap1 (n=6 ctrl, 6 Yap1 siRNA-1, 5 siRNA-2, 6 siRNA-3) or Amotl1 (n=5 ctrl, 6 Amotl siRNA-1, 6 siRNA-2, 4 siRNA-3) relative to Gapdh transcripts, in primary cultures of cardiomyocytes treated with 3 different siRNA for each gene. (g) Western blot showing efficient down-regulation of Yap1 and Amotl1 proteins relative to Gapdh in cultures treated with the indicated siRNA. (h) Corresponding quantifications (n=3 cultures in each condition). (i) Profile by flow cytometry of primary cell cultures stained with Ki67 and Tnnt2 (cardiac troponin T), using an isotype antibody as a negative control (green) or a specific primary antibody (red). (j) Number of proliferating Ki67-positive cardiomyocytes (Tnnt2-positive) after treatment with different Fat4, Yap1 and Amotl1 siRNAs (n=20 cultures for Ctrl and Fat4 siRNA-1, n=4 in each other condition). (k) Immunodetection of replicating EdU-positive cardiomyocytes when Fat4 is down-regulated, with the corresponding quantification in (l) (n=6 cultures in each condition). Ctrl, control. Scale bar: 5 μm in (b), 500 μm in (d), 10 μm elsewhere.

FIG. 8. Normal canonical Hippo signalling when Fat4 expression is impaired. (a) (m) Control of Yap1 immunodetection in cardiomyocytes treated with the indicated siRNA. (b) Western blot showing normal Yap1 phosphorylation at the Hippo kinase target site, in hearts at P0. See quantification in FIG. 3k. (c) The stability of Yap1 is decreased in Fat4^{-/-} (n=4) compared to control (n=4 Fat4^{-/+}, n=6 Fat4^{+/-}) hearts, suggesting feedback mechanisms. (d) Expression of Yap1 is not affected in Fat4^{-/-} (n=7) compared to control (n=5 Fat4^{-/+}, n=5 Fat4^{+/-}) hearts at P0. (e) Western blot of extracts of primary cultures of cardiomyocytes treated with Fat4 siRNA. (f) Normal Yap1 phosphorylation and Yap1 levels were quantified when Fat4 expression is down-regulated (n=7 cultures in each condition). (g) Western blot, quantified in (h), showing no change in the phosphorylation of the Hippo kinases Lats1,2, between Fat4^{+/-} (n=7) and Fat4^{-/-} (n=7) hearts at P0. (i) Western blot, quantified in (j), showing no change in the phosphorylation of the Hippo kinases Mst1,2 between Fat4^{+/-} (n=7) and Fat4^{-/-} (n=7) hearts at P0. Bands in the same lane of Western blots were from the same blot, retreated after washing (see FIG. S5). Ctrl, control; ns, no significant difference. Scale bars: 10 μm.

FIG. 9. Fat4 and Amotl1 expression and kinetics of the phenotype of Fat4 mutant hearts. (a) Expression of Fat4 in wild-type hearts at E10.5 (n=3), E16.5 (n=4) and P0 (n=4). Data are normalised to the level in E10.5 hearts. (b) Histological sections of control and mutant hearts at E14.5, E16.5 and E18.5. (c) Quantification of phosphorylated histone H3 (PH3)-positive cells at E10.5 (n=3 Fat4^{-/+}, 2 Fat4^{+/-}, 3 Fat4^{-/-}), E16.5 (n=2, 2, 2), E18.5 (n=3, 3, 5) and P0 (n=6, 6, 5). (d) Relative expression of the β-catenin target Snai2 in Fat4^{+/+} (n=5), Fat4^{+/-} (n=5) and Fat4^{-/-} (n=7) hearts at P0. (e) Immunodetection of active β-catenin (Ctnnb1) in Fat4^{+/+} and Fat4^{-/-} hearts at P0. (f) Quantification of the aspect ratio between the minor and major axes of cardiomyocytes indicates rounder cells in mutant Fat4^{Flox/-}; Mesp1^{Cre/+} (n=112,792 cells pooled from 6 hearts) compared to control Fat4^{Flox/+} (n=147,843 cells pooled from 7 hearts) samples at P14. (g) Relative transcript levels of genes of the angiomotin family in wild-type hearts (n=4) at E16.5. Scale bars: 500 μm.

FIG. 10. Original un-cropped Western blots.

FIG. 11. Nuclear Yap1 is not sufficient to promote the proliferation of neonate cardiomyocytes.

FIG. 12. The proliferative effect of nuclear Amotl1 is dependent on Yap1.

FIG. 13. Yap1 follows Amotl1 in cells. Colocalisation at the membrane of cultured caridomyocytes. The vesicular localisation of Amotl1 titrates Yap1 and prevents its nuclear translocation. The nuclear translocation of Amotl1 increases nuclear Yap1.

FIG. 14. How to use Amotl1 to stimulate cardiomyocyte proliferation? Amotl1 is required for cell proliferation. Amotl1 overexpression is not sufficient for its nuclear translocation.

FIG. 15. The Pdz binding domain is not involved in the sequestration of Amotl1 out of the nucleus.

FIG. 16.Phosphorylation by Lats2 is not involved in the sequestration of Amotl1 out of the nucleus.

FIG. 17. Amotl1 is sequestered out of the nucleus via its N-terminal domain.

### EXAMPLES

1. Material and Methods

1.1. Animal Models

The Fat4 mouse mutant line⁸ was maintained in a 129S1 genetic background. Fat4 conditional mutants⁸ were crossed to Mesp1^{Cre/+ 30}, Wt1^{Cre/+ 31} lines or Yap conditional mutants³² and backcrossed in the 129S1 genetic background. Fat4^{-/-} mutants die at birth, whereas Fat4^{flox/-}; Mesp1^{Cre/+} survive. Animal procedures were approved by the ethical committee of the Institut Pasteur and the French Ministry of Research. For histological analysis, hearts were excised, incubated in cold 250 mM KCl, fixed in 4% paraformaldehyde, embedded in paraffin in an automated vacuum tissue processor and sectioned on a microtome (10 μm). For immunofluorescence studies, hearts were fixed in 0.5% paraformaldehyde, embedded in gelatine/sucrose, frozen in cold isopentane and sectioned on a cryostat (10 μm). For the quantification of tissue growth, paraffin sections stained with Hematoxylin Eosin were imaged on a stereomicroscope. A polygonal mask was drawn in order to isolate the two ventricles from the atria. The green channel (with highest contrast) of the resulting image was inverted, thresholded and segmented using Connected Component analysis. The resulting regions were sorted, retaining the myocardial tissue and excluding blood speckles inside the ventricles, to compute the total area of the ventricles. The penetrance of the myocardial excessive growth was 75% (n=8). Unless otherwise specified, the image analysis was done using the Icy software³³.

1.2. RT-qPCR

cDNAs were reverse transcribed from RNAs extracted in TRIzol from cell cultures and isolated hearts using the Quanti-Tect kit (Qiagen) and Superscript II Reverse Transcriptase (Life Technologies) respectively. Quantitative PCR was carried out on a StepOne System (Life Technologies) using Fast Start SYBR Green Master (Roche). Quantification of gene expression was calculated as R=2^{ΔCt(control-target)}, with Gapdh used as a control. Primers were designed using the NCBI Primer-BLAST software. Primer sequences are listed in Supplementary Table 1.

SUPPLEMENTARY TABLE 1
List of primer sequences used in RT-qPCR.

| Species | Gene | Forward Primer | Reverse Primer |
|---|---|---|---|
| Mouse | Acta1 | GCATGCAGAAGGAGATCACA | ACATCTGCTGGAAGGTGGAC |
| Mouse | Amot | ACTGAGGGTCCTGCAAATCC | CAGGGGCATCTGGCTTATCT |
| Mouse | Amotl1 | CCCGCCTACTTCTACCCAGA | GGGTCCTCTACGCTTTTCCC |
| Mouse | Amotl2 | AAACTGCTTGCCCAGAGCTA | TCCAGCAGTTCAGCATGTCG |
| Mouse | Aurkb | GATTGCAGACTTTGGCTGGTC | ATTTCATTATGCATGCGCCCC |
| Mouse | Birc2 | TGAGAACTACAGGACCGTCAAT | TCTTCCGAATCAGTGATAAGTCA |
| Mouse | Birc5 | GAACCCGATGACAACCCGAT | TGGCTCTCTGTCTGTCCAGT |
| Mouse | Ccna2 | CCCGGAGCMGAAAACCACT | TCATTAACGTTCACTGGCTTGT |
| Mouse | Cdc20 | CGCATTTGGAACGTCTGCTC | GCAAAGCCGTGACCTGAGAT |
| Mouse | Cdkn1b | GTTTCAGACGGTTCCCCGAA | CTTAATTCGGAGCTGTTTACGTCT |
| Mouse | CTGF | AGAACTGTGTACGGAGCGTG | GTGCACCATCTTTGGCAGTG |
| Mouse | Cyr61 | TTGACCAGACTGGCGCTCTC | AGCGCAGACCTTACAGCAG |
| Mouse | Fat4 | AGGACTTTGGTGGCATTGAG | GGGTCTGTTTTGGAGATGGA |
| Mouse | Gapdh | ACCCAGAAGACTGTGGATGG | CACATTGGGGGTAGGAACAC |
| Mouse | Myh6 | CAAGCTGCAGACAGAGAACG | TGCTGGGTGTAGGAGAGCTT |
| Mouse | NppA | ATCTGCCCTCTTGAAAAGCA | GCTCCAATCCTGTCAATCCT |
| Mouse | Nppb | TCGGATCCGTCAGTCGTTTG | TTCAAAGGTGGTCCCAGAGC |
| Mouse | Snai2 | ACTGGACACACACACAGTTAT | TGCCGACGATGTCCATACAG |
| Rat | Amotl1 | ACAAAGCTGCAGAGAGCCAT | TGCCAGCTCCATTTCCAACT |
| Rat | Fat4 | GGGGACAGATGTCCTGTTGG | TGAACTGTGAGTTTCCACCGA |
| Rat | Gapdh | AAGTTCAACGGCACAGTCAAG | TACTCAGCACCAGCATCACC |
| Rat | Yap1 | CTGCCCGACTCCTTCTTCAA | TGGAGACGAGTGAGCTCGAA |

1.3. Primary Cell Culture

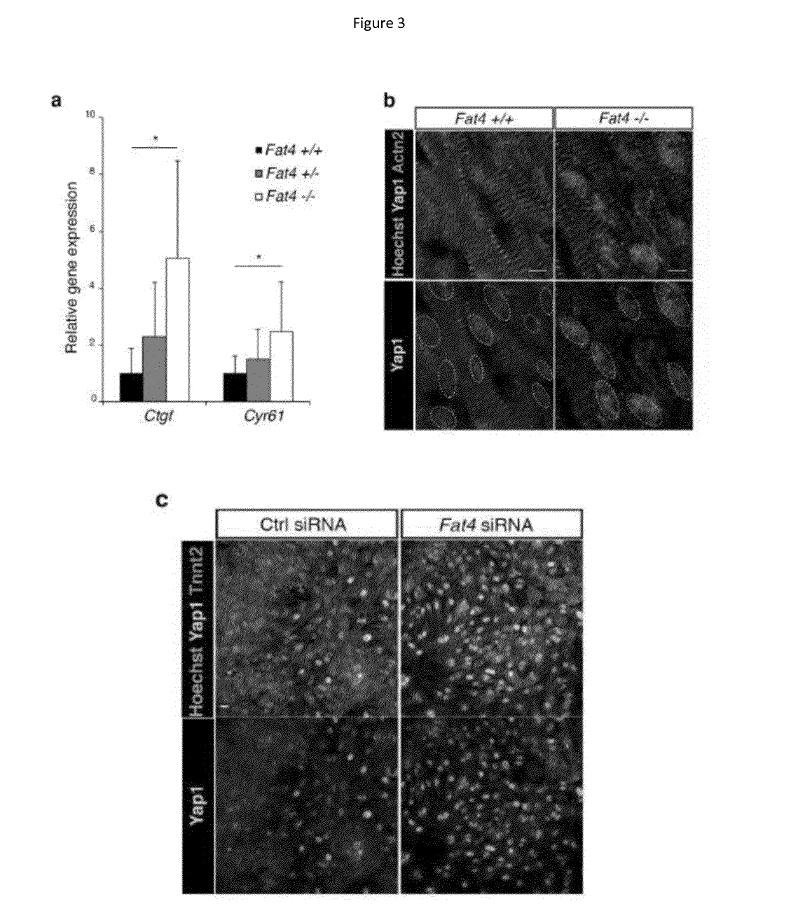

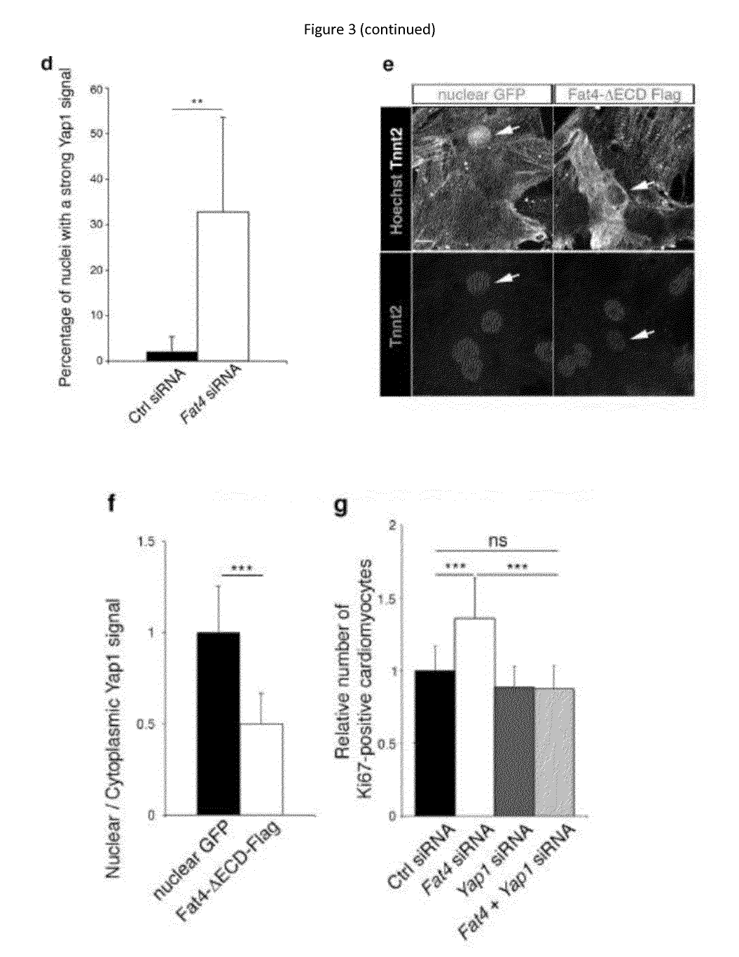

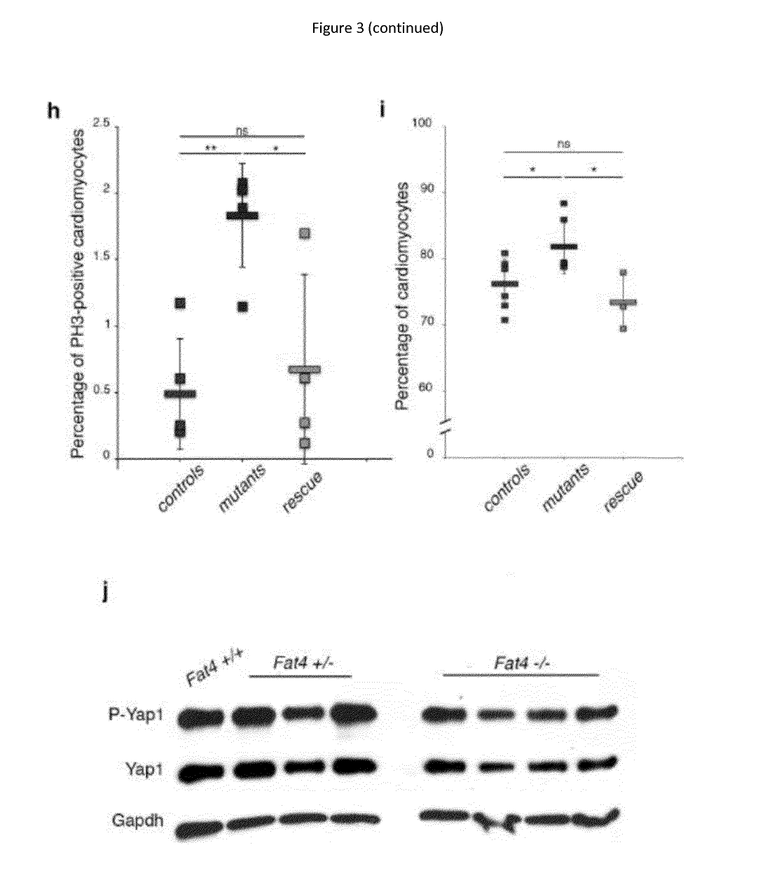

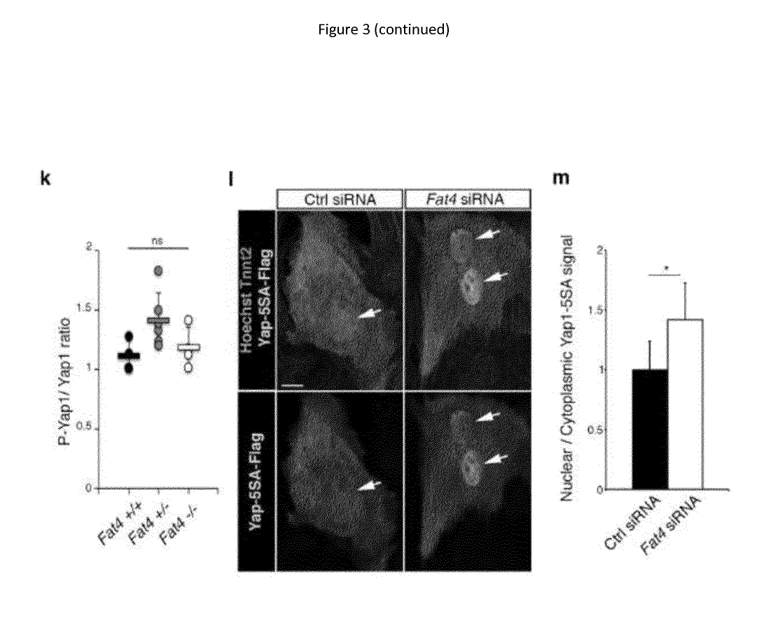

Primary cultures of newborn rat cardiomyocytes, performed as previously described³⁴, were transfected with siRNA at 10 pM using Lipofectamine RNAiMax with silencer-siRNA at 24 h and analysed at 72 h, or 96 h (FIG. 3c). Control (Ambion 4390843), Fat4 (siRNA-1: Ambion s172170, siRNA-2: CCUGUACCCUGAGUAUUGATT, siRNA-3 CCGUCCUUGUGUUUAACGUTT), Amotl1 (siRNA-1: AUCUCUACCAUUUGUUGGGTT, siRNA-2: GAGUAUCUCAGAGGCCUAUTT, siRNA-3: CAUCACAUGUCCCAGAAUATT), Yap1 (siRNA-1: Ambion s170200, siRNA-2: GUCAGAGAUACUUCUUAAATT, siRNA-3: GGAGAAGUUUACUACAUAATT) siRNA were used. Efficiency of the interference was controlled by RT-qPCR. In the figures, Fat4 siRNA is Fat4-siRNA-1, Yap1 siRNA is a pool of siRNA-1 to 3 and Amotl1 siRNA is a pool of siRNA-1 to 3.

For flow cytometry analyses, cultures were dissociated to single cell suspensions by trypsin, fixed and permeabilized in eBioscience buffer. Proliferating cardiomyocytes were detected by immunostaining with primary antibodies against Tnnt2 (ab64623) and Ki67 (BD 556027) and counted on a BD LSRFortessa Cell Analyzer cytometer. Gates were set according to isotype control antibodies (sc-3887). At least 900 cells were counted per condition. Alternatively, cardiomyocytes were exposed to EdU during 30 h and counted after immunofluorescence (at least 80 cells per condition). For overexpression experiments, cardiomyocytes were transfected using Lipofectamine 2000 with Fat4-DECD-Flag (encoding Fat4 depleted for the extracellular domain and for the last C-terminal 297 nucleotides, CB and HMN, unpublished data), HA-Amotl1²⁴, Yap1-5SA (Addgene 27371) or control nuclear GFP (pCIG³⁵) plasmids and analysed 24 h later. Alternatively, cardiomyocytes were infected with adenoviruses at a multiplicity of infection of 50 and analysed 24 h later, using control Ad-GFP³⁶ or newly generated HA-(nls)₃-Amotl1. It was cloned from human Amotl1³⁷ in the Adeno-X Expression System 3 (Clontech).

1.4. Immunofluorescence

Immuno fluorescence was performed as previously described¹⁸, using primary antibodies to acetylated tubulin (Sigma T6793), Actn2 (Sigma A7811), Amotl1 (Sigma HPA001196), Amotl1 (Covalab, gift from D. Lallemand), Aurkb (BD 611082), non-phosphorylated (Ser^{33/37}-Thr⁴¹) β-catenin (Ctnnb1, Cell signalling 8814), Cav3 (BD 610420), Cdh2 (Ab12221), Ki67 (BD 556003), MF20 (DSHB), PH3 (ab32107), Pkp2 (Progen 651167), Scrib (sc-28737), Tnni3 (ab47003), Tnnt2 (ab64623), Vcl (Sigma F7053), Yap1 (sc-101199 and sc-15407), HA (Roche, 3F10), Flag (Sigma, F7425), Alexa Fluor conjugated secondary antibodies and Hoechst nuclear staining Multi-channel 16-bit images were acquired with a Leica SP5 inverted confocal microscope and a 40/1.25 oil objective or with a Zeiss LSM 700 microscope and a 63×/1.4 oil objective.

Quantification of Cell Proliferation

The PH3 channel was thresholded and segmented using Connected Component analysis, filtering objects under a minimum size of 16 μm³ in order to eliminate non-specific signals. The myocardial volume of the multi-z scan was estimated by manually outlining the myocardial surface in the median Z-slice and computing the area. The total number of cardiomyocyte (a-actinin-positive) nuclei in the scan was estimated by manually counting the number of nuclei in a 200 pixels×200 pixels window extending over all the Z-slices, and extrapolating to the total myocardial volume. More than 1,500 nuclei were counted per genotype.

Quantification of Cell Size

Images of cardiomyocyte transverse sections labelled with Caveolin3 (Cav3) were acquired systematically in the interventricular septum. Cell contours were drawn manually to compute cell area using ImageJ. At least 40 cells were counted per genotype.

Quantification of Protein Localisation

The best in-focus Z-slice of the Hoechst channel was chosen for in vivo cells, whereas in vitro images were Z-projected. The analysis involved three image processing steps: 1) Segmentation of the myocardial (Tnnt2-positive) cells using Connected Component analysis applied after a Z-projection (sum) and thresholding of the Tnnt2 channel; alternatively, in vitro transfected cells were individually outlined manually; 2) Segmentation of the nuclei by thresholding after application of a Gaussian filter (in vivo), or by the “Active Contours” plugin (in vitro); 3) Measurement of the total intensity of the protein of interest (PI) in the Tnnt2-positive cells (PIₜₒₜ) and in their nuclei (PI_{nucl}) by multiplication of the PI channel with the respective binary images (1) and (2). Strong cells were defined as cells in which PI_{nucl} is higher than two standard deviations above the mean PI_{nucl} of control cells. The nuclear/cytoplasmic ratio was computed as: PI_{nucl}/PI_{cyto}=PI_{nucl}/(PIₜₒₜ−PI_{nucl}). For in vivo cells, which have a more pronounced 3D shape, total intensities were divided by the area of the segmented object. At least 200 cells were counted per condition.

1.5. Immunoprecipitation and Western Blots

HEK293 cells (Q-BIOgene AES0503) were transfected with Lipofectamine with the plasmids Amotl1-HA²⁴ and Flag-Fat4-ΔECD and cultured for 48 h. Proteins were extracted in a lysis buffer (150 mM NaCl, 5 mM EDTA, 10 mM Tris pH 7.5, 10% glycerol, 1% NP-40) in the presence of protease inhibitors. Immunoprecipitation of protein extracts was performed using a standard protocol based on magnetic beads coupled to bacterial protein G, an immunoglobulin-binding protein. Proteins were eluted in Laemmli buffer. An isotype antibody (IgG) was used as a negative control of immunoprecipitation.

Proteins from cell cultures and isolated hearts were extracted for western blots in RIPA (150 mM NaCl, 5 mM EDTA, 50 mM Tris pH 7.4, 0.1%SDS, 1% NP-40) and NP40 (150 mM NaCl, 50 mM Tris pH 8, 1% NP-40) buffers, respectively, in the presence of protease and phosphatase inhibitors. Equal amounts of proteins were separated on SDS-PAGE and transferred to nitrocellulose or PDVF membranes. Proteins were detected with the primary antibodies Flag (Sigma F1804), Gapdh (Cell signalling 3683), HA (Roche 3F10), Thr^{1079/1041} Phospho-Latsl/2 (Assay Biotech ref A8125), Lats11/2 (Bethyl A300-478A), Thr^{183/180} Phospho-Mst1/2 (Cell signalling 3681), Mst1 (Cell signalling 3682), Mst2 (Cell signalling 3952), Ser¹²⁷ Phospho-Yap1 (Cell signalling 4911),Yap1 (Cell signalling 4912) or Amotl1 (Sigma, HPA001196), followed by HRP-conjugated secondary antibodies (Jackson ImmunoResearch) and the ECL2 detection reagent. Protein quantification was obtained by densitometry analysis using a Typhoon laser scanner and normalized to Gapdh levels. Original un-cropped blots are shown in FIG. 10.

1.6. Image Registration

In order to obtain a full inferior view of the two ventricles at E10.5, confocal scans of the left ventricle, interventricular region and right ventricle were stitched together. The envelopes of the stitched images were computed by Active Mesh segmentation³⁸. Ten such envelopes were used to compute an average envelope (referred to as the template), minimising the deformation distances between the template and the envelopes, plus a residual mismatch cost. The metric distance was built on a group of smooth invertible deformations (i.e. diffeomorphisms³⁹). The axial data from each image were then transported through the deformation between the original envelope and the template, as described by the Jacobian matrix of the diffeomorphism (i.e. the matrix of partial derivatives of the deformation, a 3D generalization of the gradient). Using the polar part of the Jacobian was required to avoid improvement of the axial correlation.

1.7. Quantification of Tissue Polarity

Whole mount immunostaining was carried out on E10.5 isolated hearts using Scrib and Cadh2 antibodies to detect membranes and Aurkb antibody to detect cytoplasmic bridges. The nuclei and cytoplasmic bridges were segmented, sister cells were automatically detected and the axes of cell division were calculated as previously described^{18,40}. For each genotype, at least three E10.5 embryonic hearts were registered, in order to pool the axial data on a common template. The planar component of each axis of cell division was extracted by projection over the template envelope. The threshold eigenvalue for each region size, E_{1(5%)}, which was obtained by a bootstrap method¹⁸, was calculated both before and after the diffeomorphic transport of the axes, and the highest value was retained to compensate for any spurious improvement of the alignment due to the transport. Contour maps of axial coordination were produced as follows: 1) Selection of the region, containing at least 50 axes, with the highest eigenvalue E₁ (core region); 2) Listing all regions that both included the core region and had an eigenvalue E₁>E_{1(5%)}; 3) Drawing these regions on the template, with contour values equal to the ratio E_{1/}E_{1(5%)}.

1.8. Electron Microscopy

Neonate hearts were dissected in cold Krebs buffer without calcium, and fixed open with 2% glutaraldehyde in cacodylate buffer (Na Cacodylate 150 mmol/L, CaCl2 2 mmol/L, pH 7.3). The left ventricular papillary muscles were excised and fixed again in 2% gluteraldehyde in cacodylate buffer, post-fixed in 1% OsO4, contrasted in 1% uranyl acetate, dehydrated and embedded into Durcupan.Ultrathin (58-60 nm) longitudinal sections were cut by Power-Tome MT-XL (RMC/Sorvall, USA) ultramicrotome, placed on copper slot grids covered with formwar and stained with lead citrate. The sections were examined in a JEM 2000FX (Jeol, Japan) electron microscope and recorded using a Gatan DualVision 300W CCD camera (Gatan Inc., USA).

1.9. ImageStream

P14 hearts were collected, minced and flash frozen as previously described⁴¹. The defrosted tissue was fixed in 4% paraformaldehyde, digested with 3 mg/ml collagenase type II in HBSS and filtered using a 100 μm cell-strainer. Staining of isolated cells was performed with the BD Cytofix/Cytoperm Fixation/Permeabilization Kit, using anti-sarcomeric α-actinin (Sigma) and DRAQ5 nuclear stain. Data acquisition was performed using an ImageStreamX cytometer with INSPIRE software (Amnis). Files were collected with a cell classifier applied to the brightfield channel to capture events larger than 100 μm. At least 23,000 cell events were acquired for each sample and all images were captured with the 40× objective. Data analysis was performed with IDEAS software (v6.0, Amnis). Images were compensated using a matrix generated by single-stained samples acquired with identical laser settings in the absence of brightfield illumination. The analysis was restricted to in-focus single cells and to intact cardiomyocytes, selected as actinin and DRAQ5 double positive. An object mask was created on the brightfield channel and the aspect ratio was defined as the ratio between the minor and major cell axis. The number of nuclei per cell was assessed using the DRAQ5 images, in at least 350 cells per heart.

1.10. Statistics

Sample size was chosen in order to ensure a power of at least 0.8, with a type I error threshold of 0.05, in view of the minimum effect size that was looked for. The sample size was calculated using the observed variance of the wild-type mice for the phenotype considered. Sample outliers were excluded according to the Thompson Tau test. The experiments were not randomized and the investigators were not blinded to allocation during experiments and outcome assessment.

Comparisons of center-values were done on either the average or the geometrical mean when ratios were compared. An ANOVA was systematically calculated when more than two center-values were compared, and Tukey-Kramer's test was used for the assessment of bilateral significance. Otherwise, a Student test was used. When n>10, normality was checked by a Kolmogorov-Smirnov test or by visualisation of the distribution. When the test was not positive, a Wilcoxon U test was used.

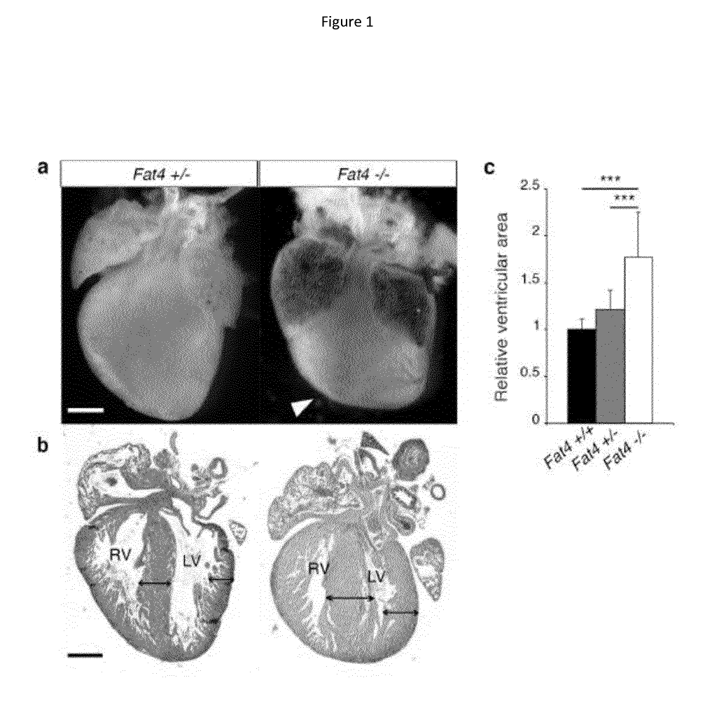

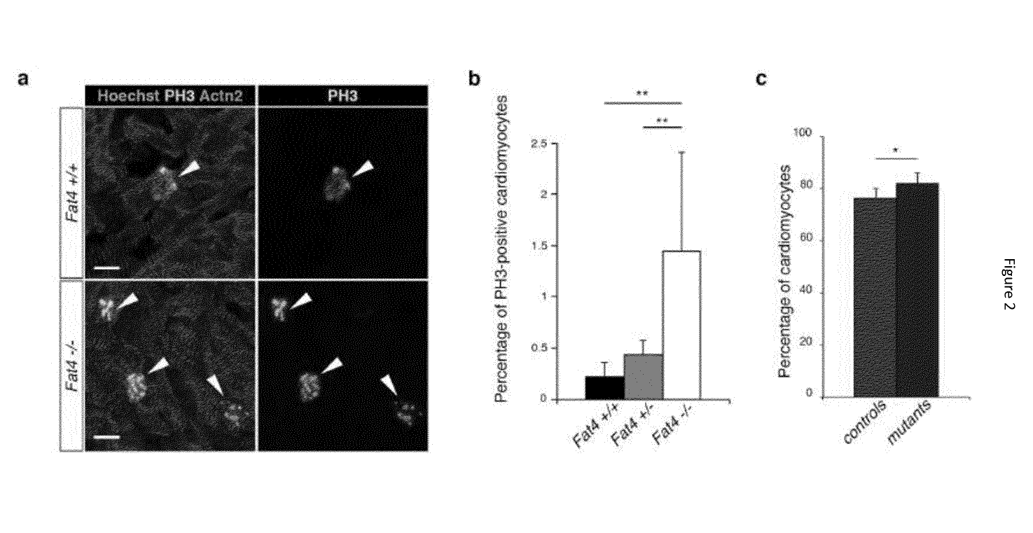

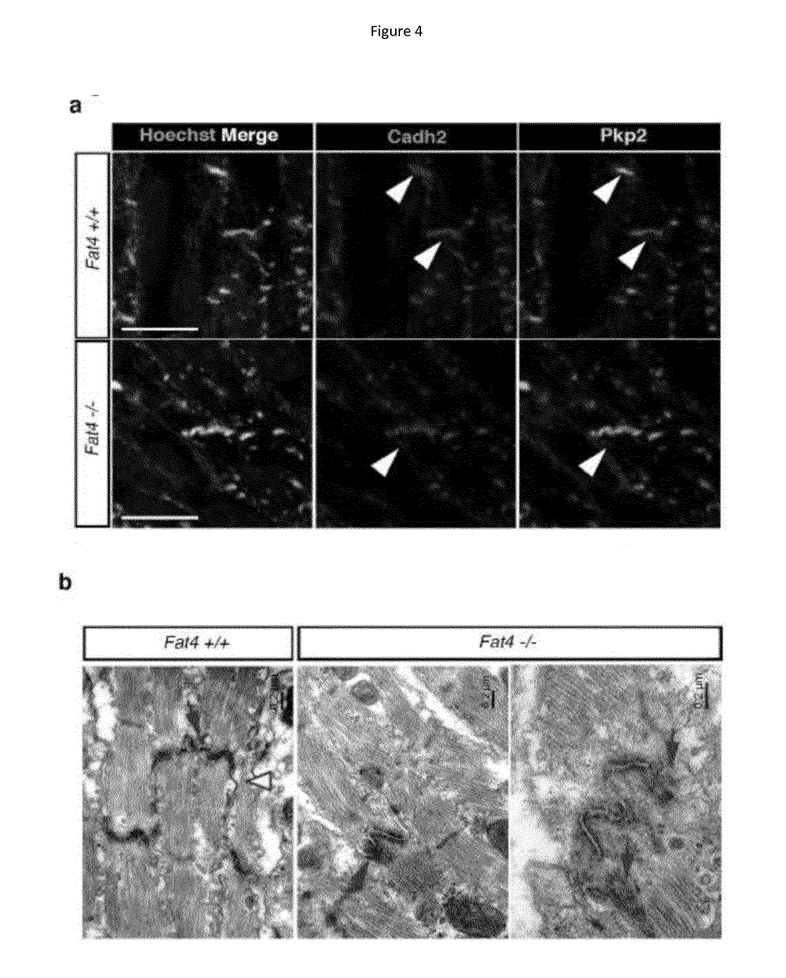

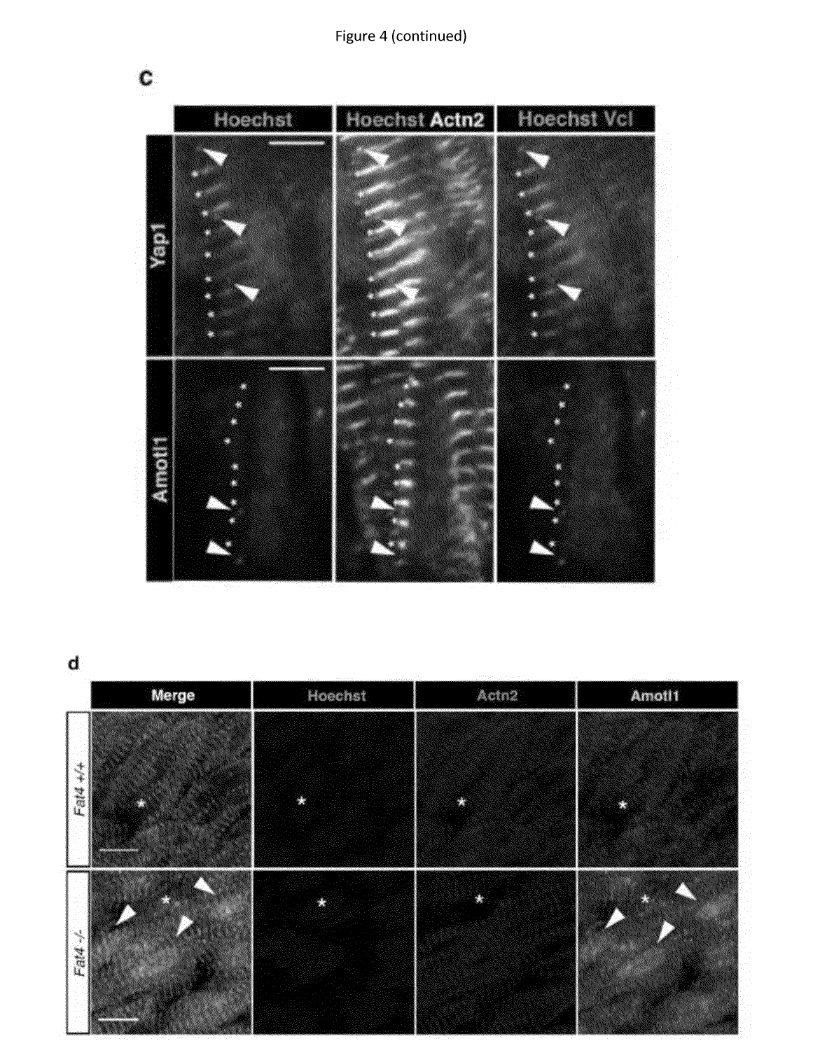

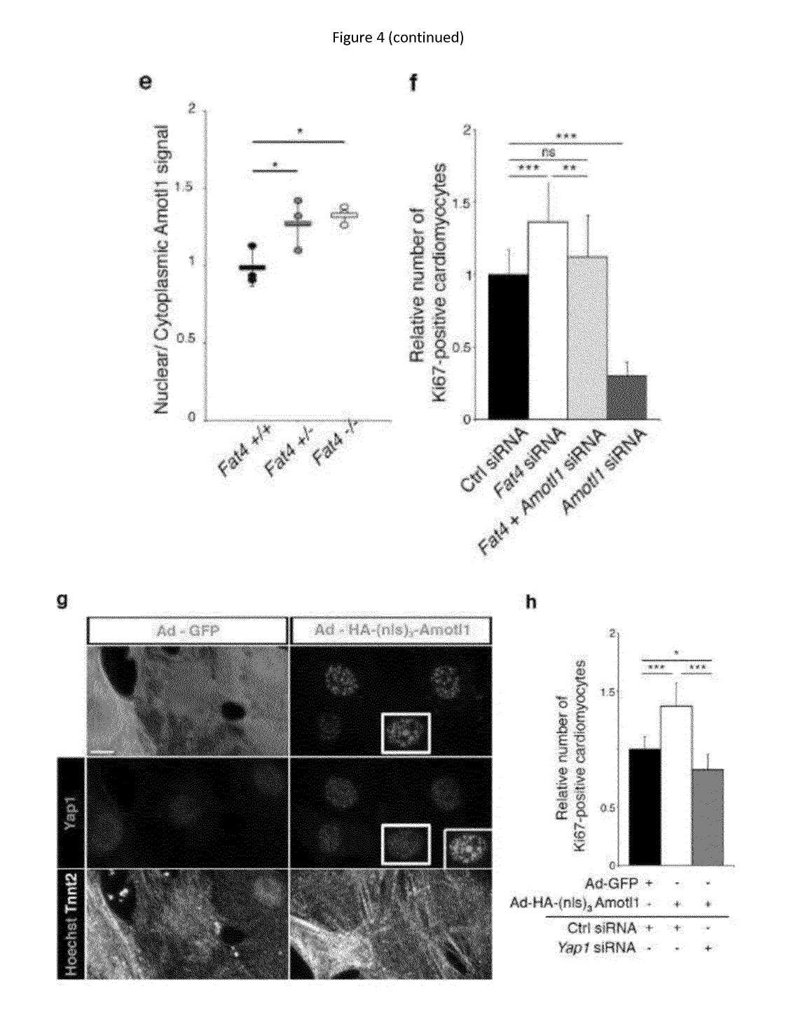

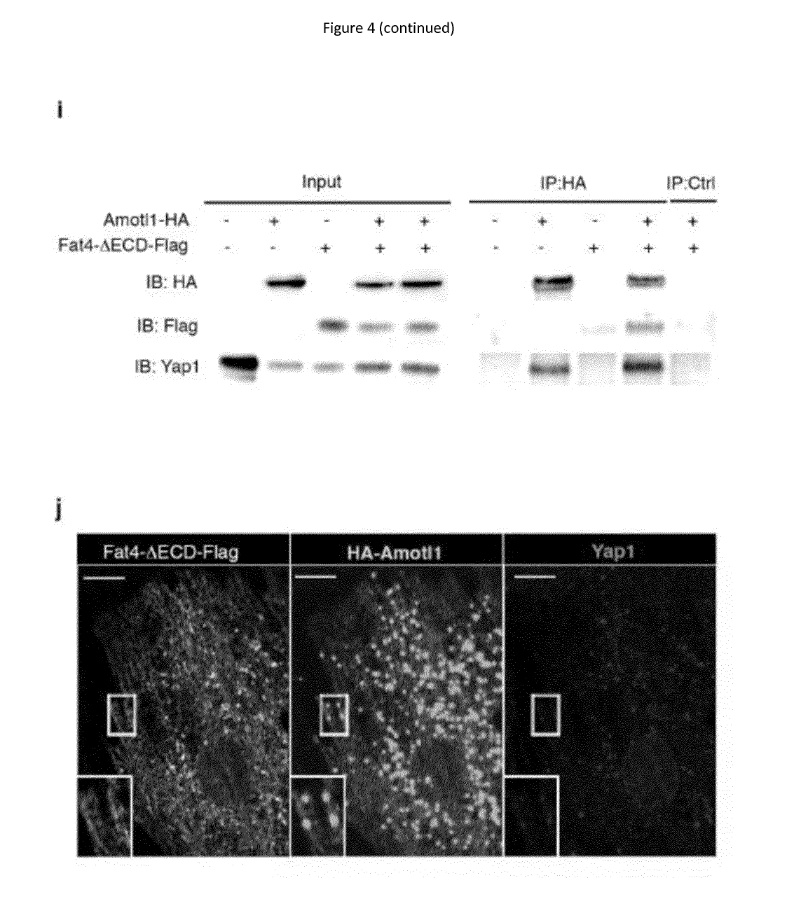

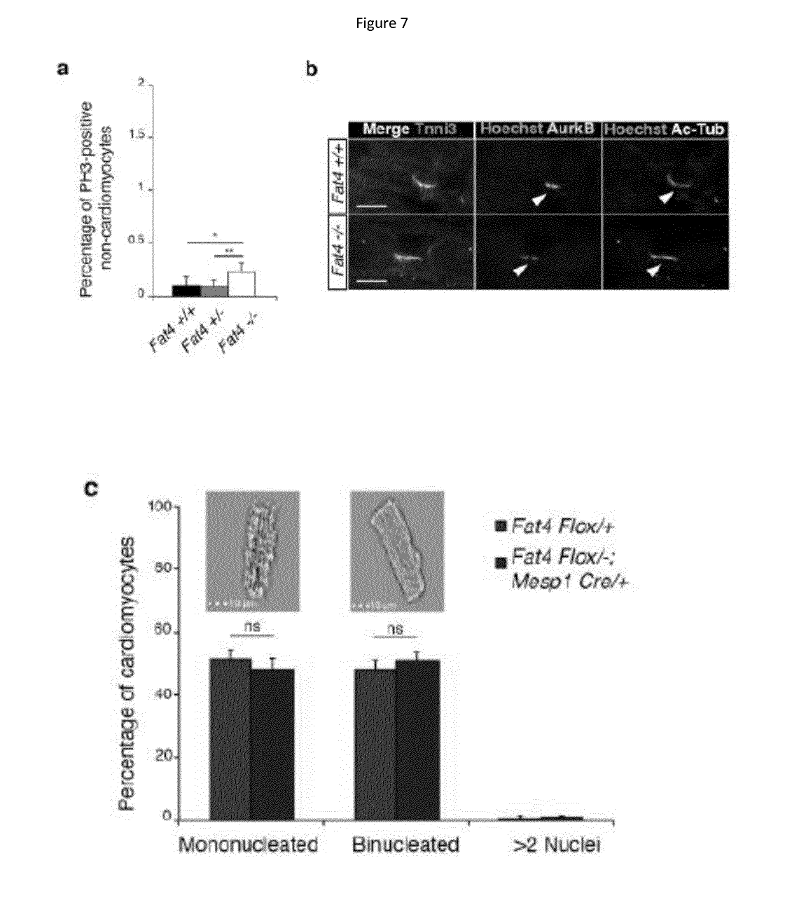

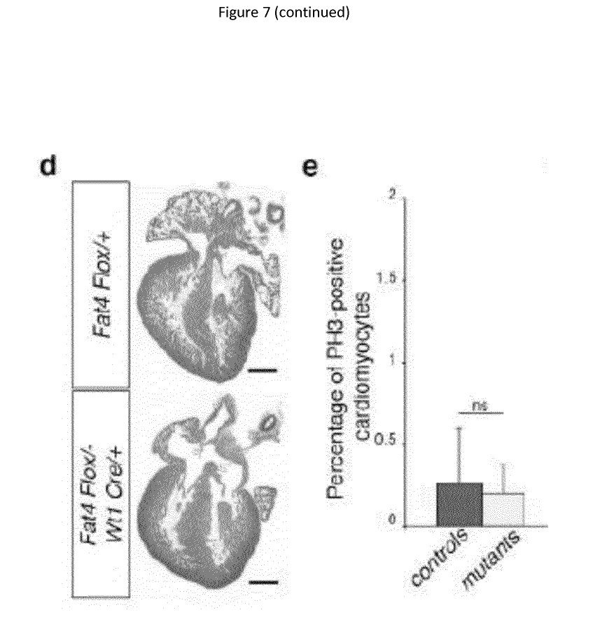

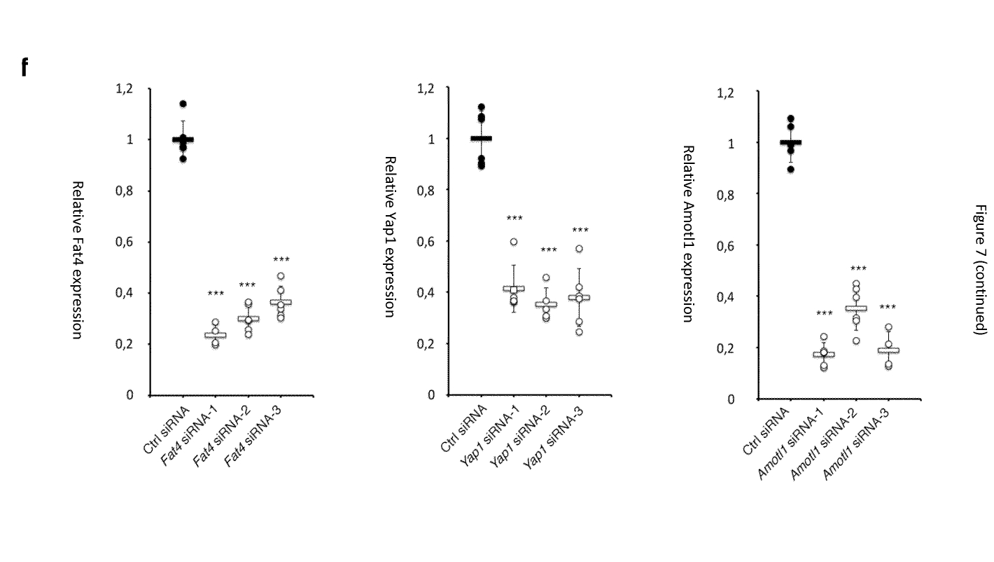

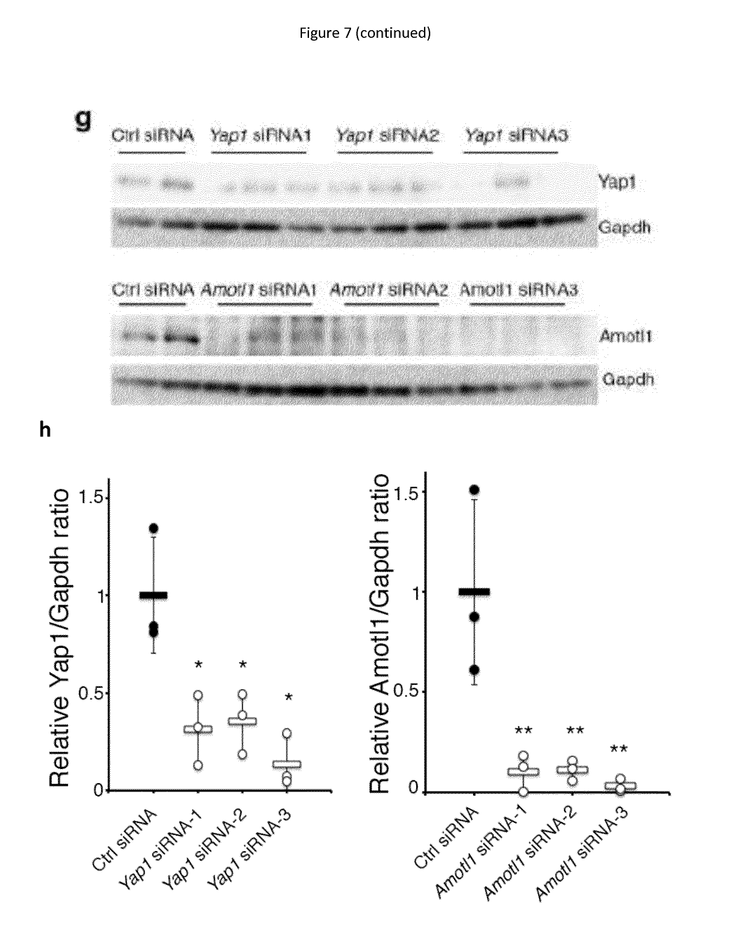

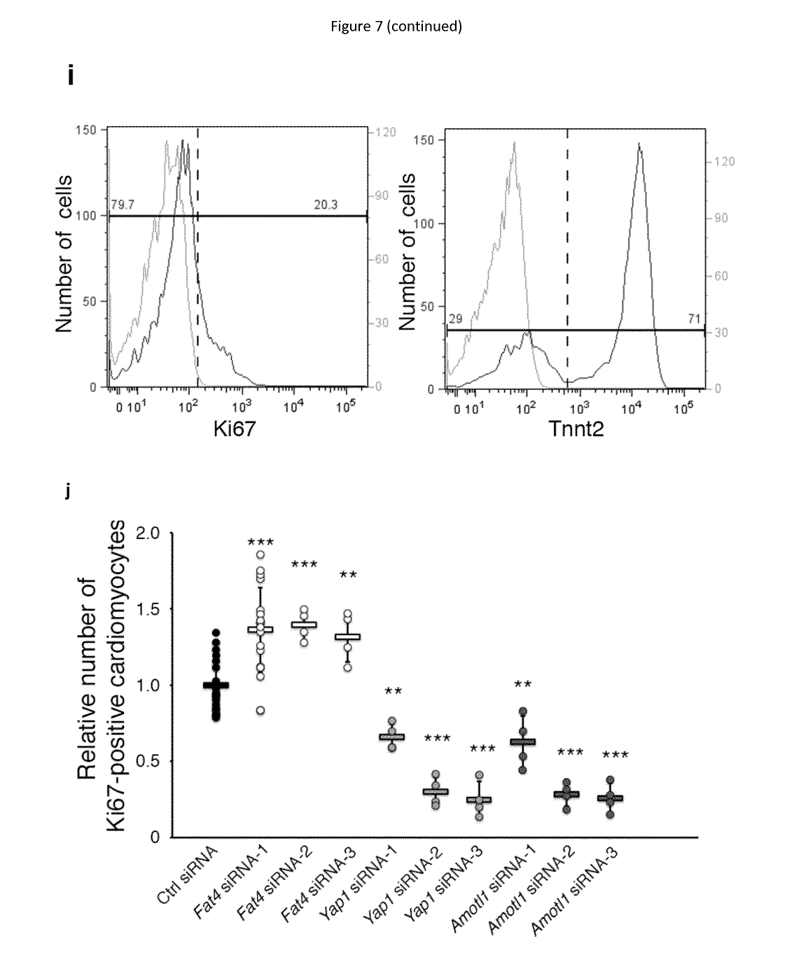

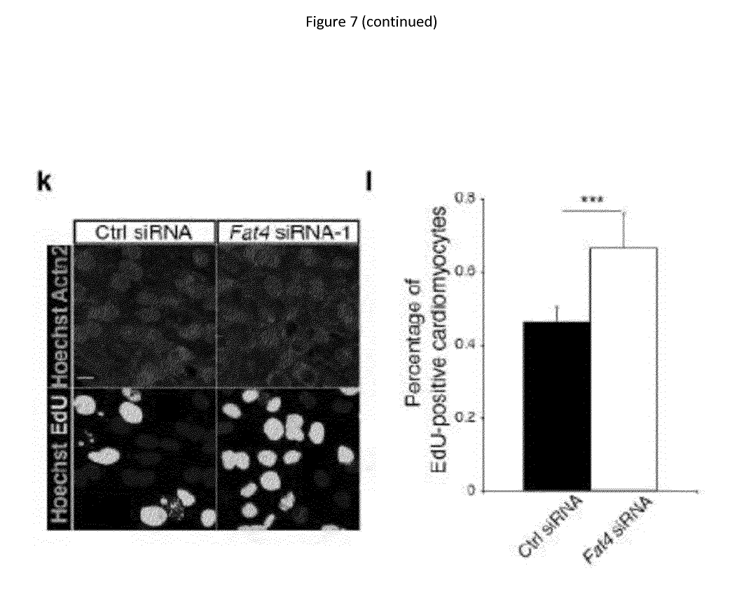

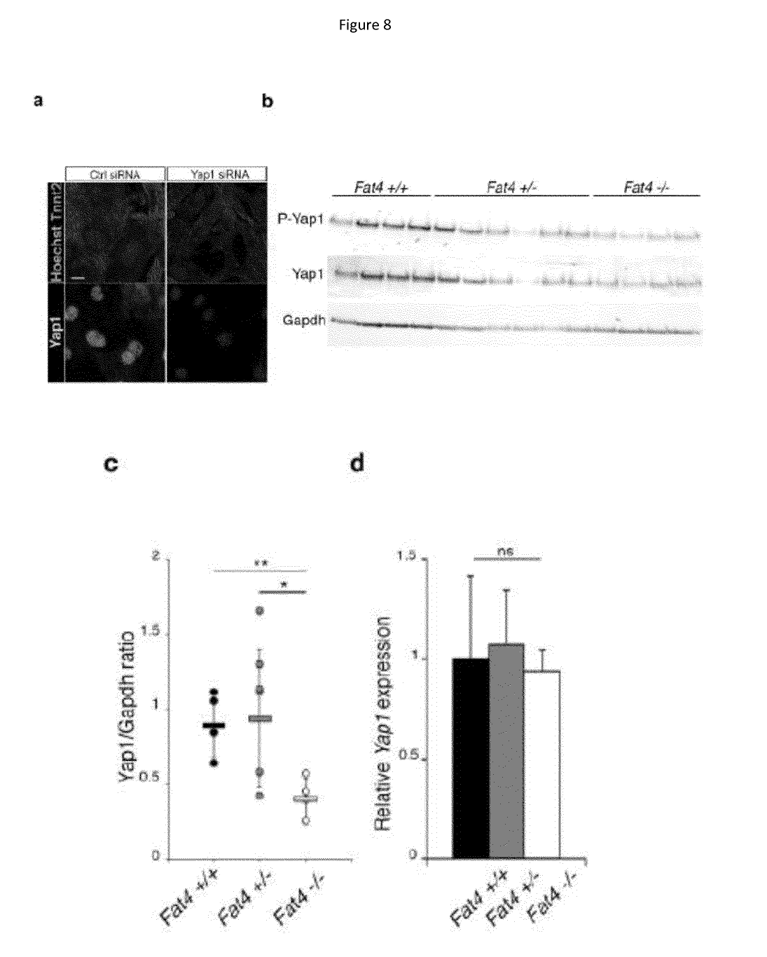

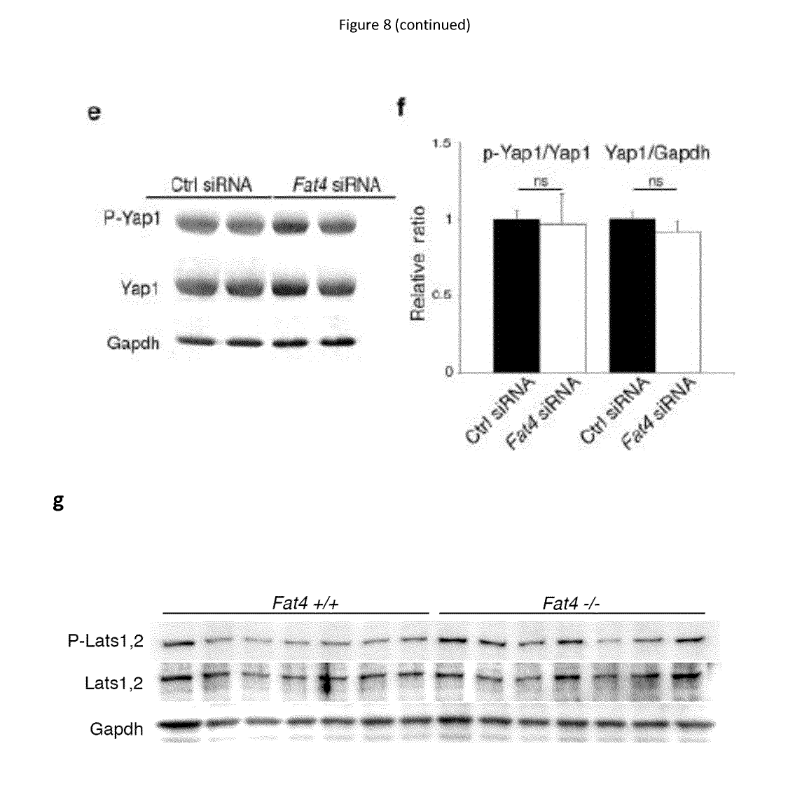

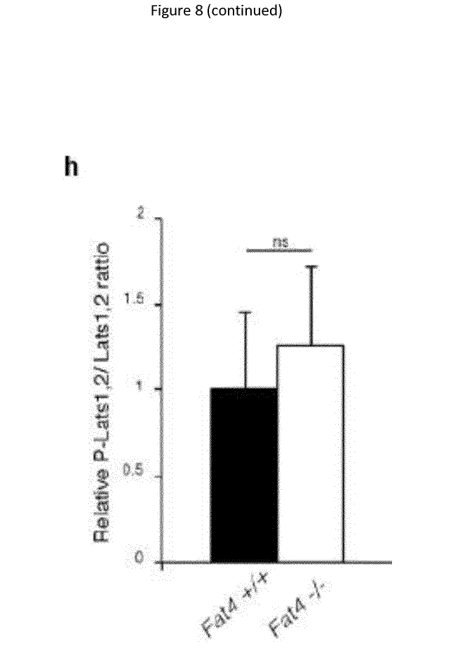

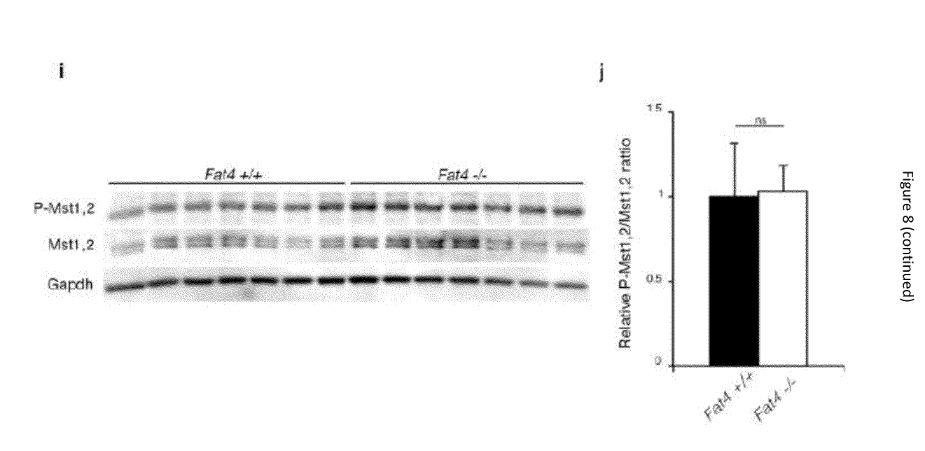

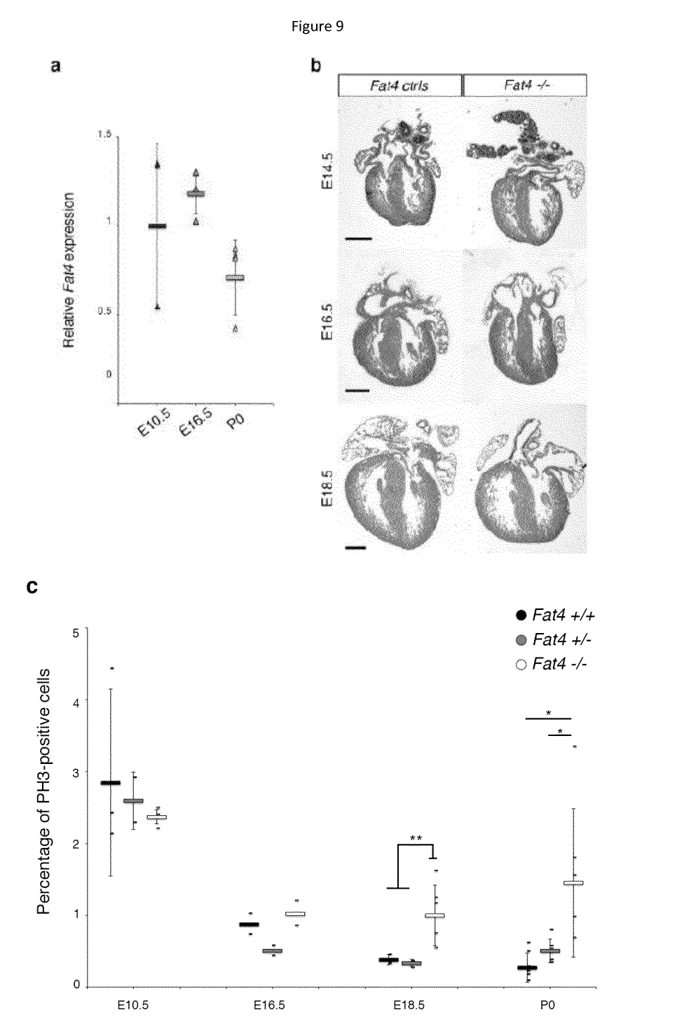

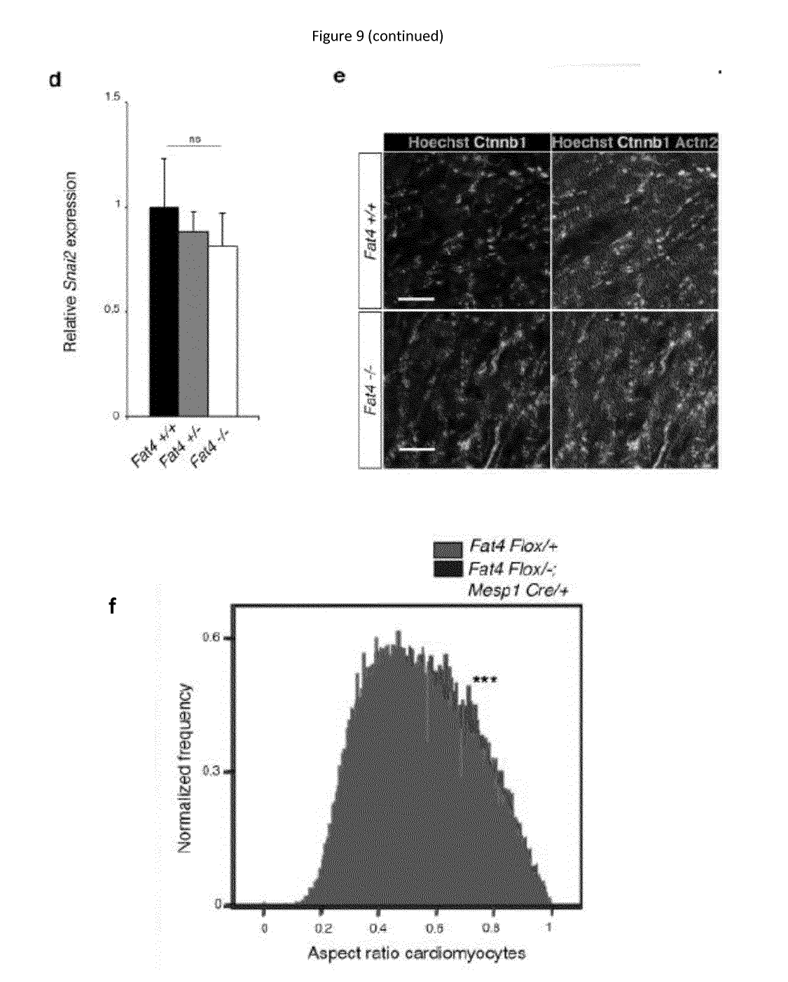

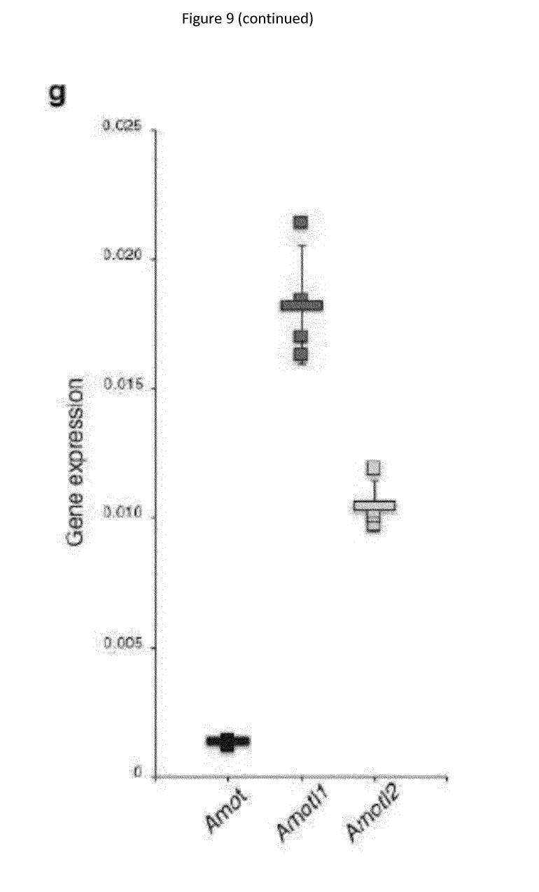

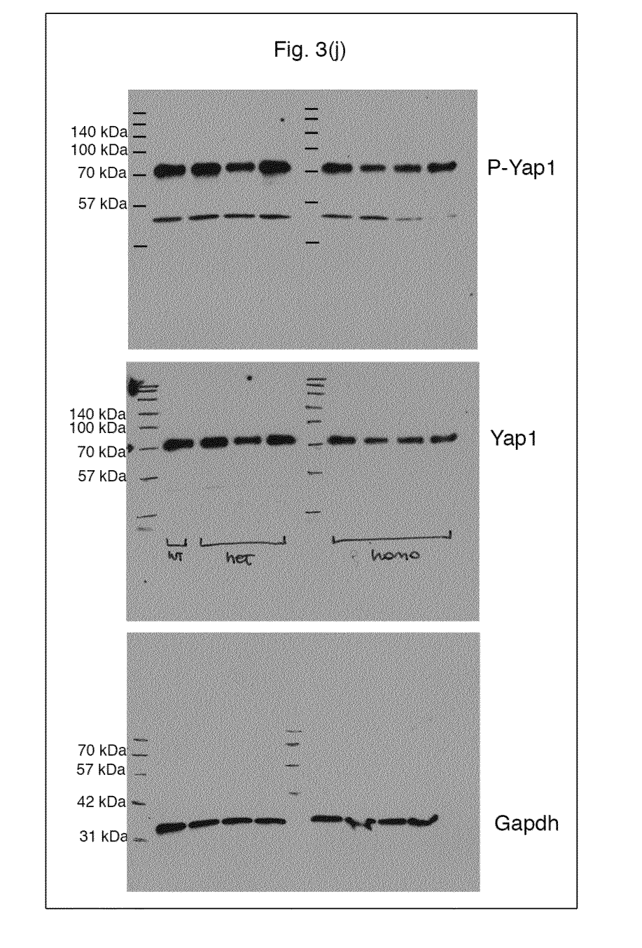

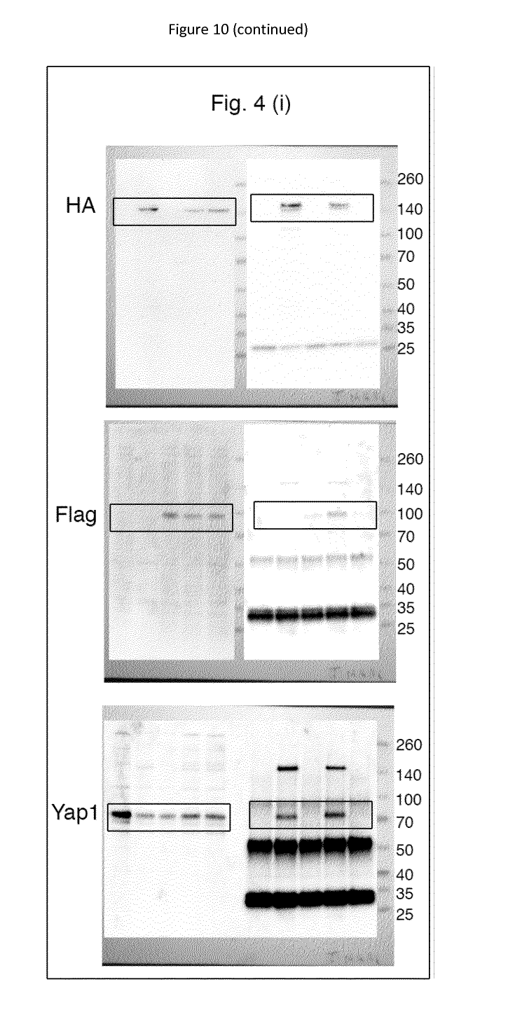

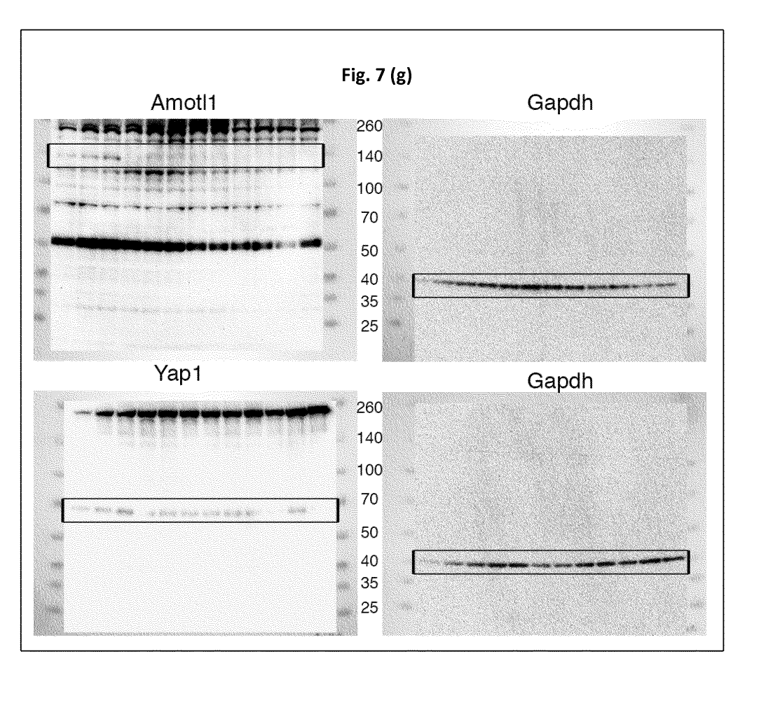

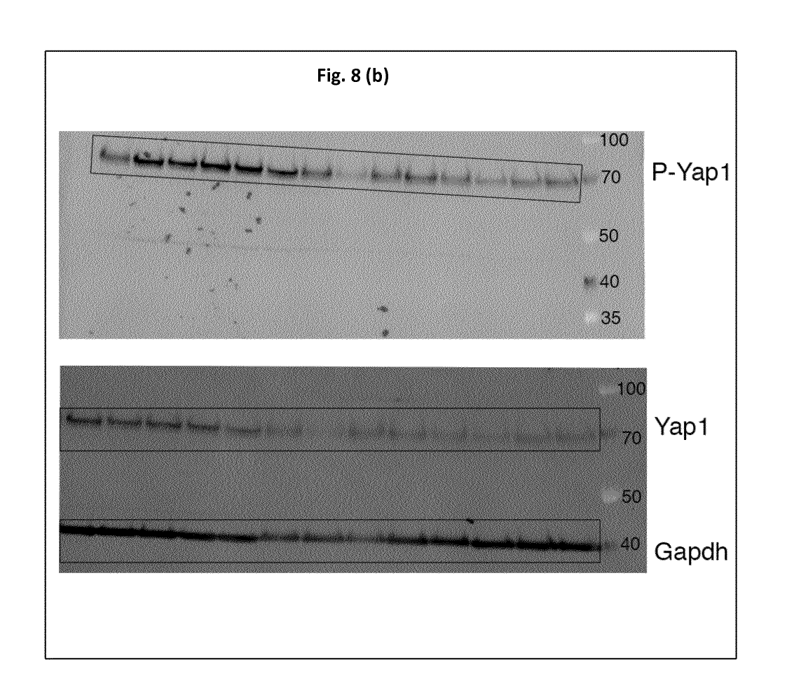

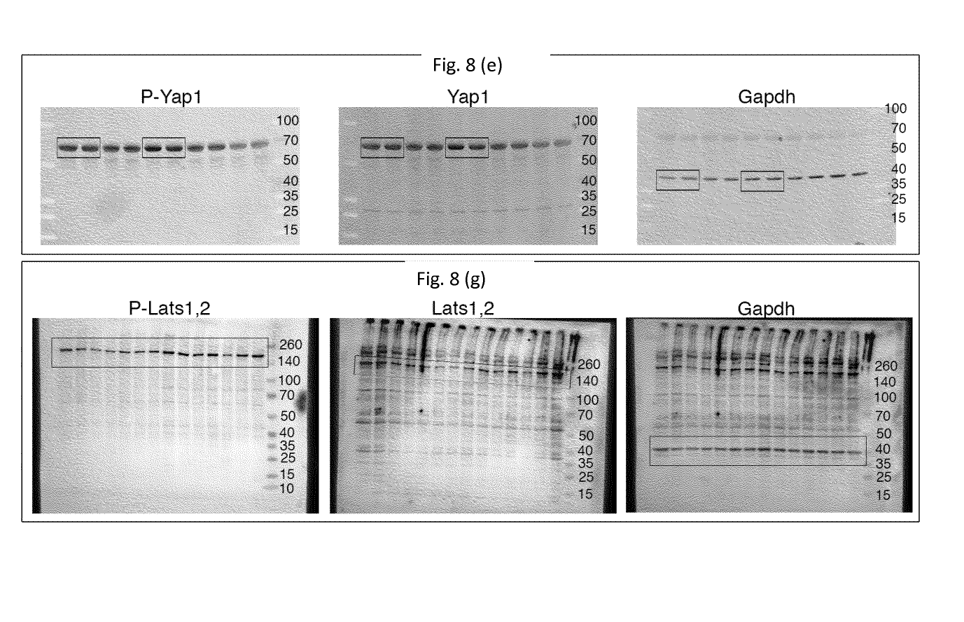

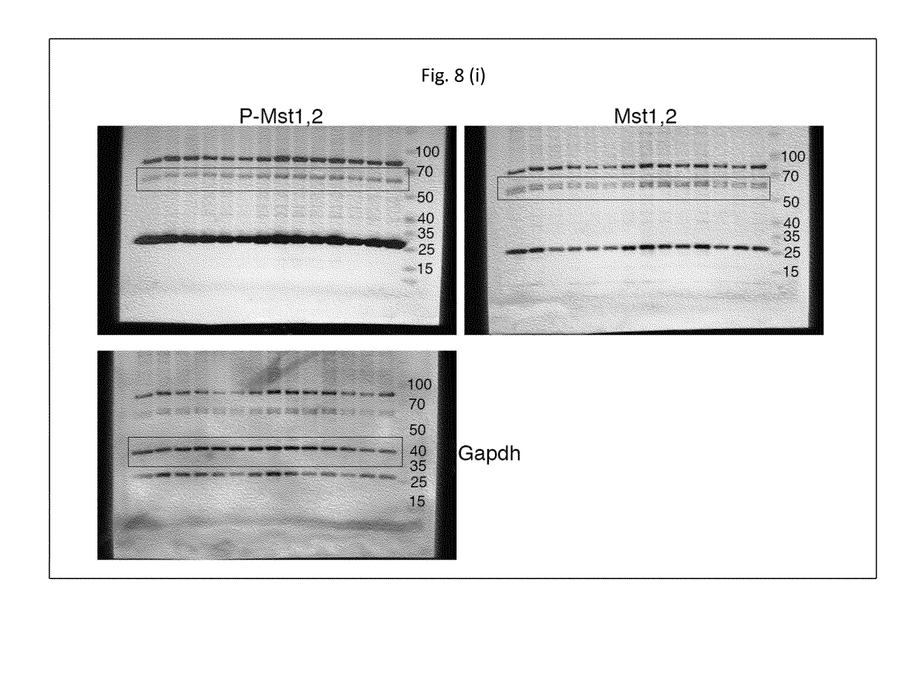

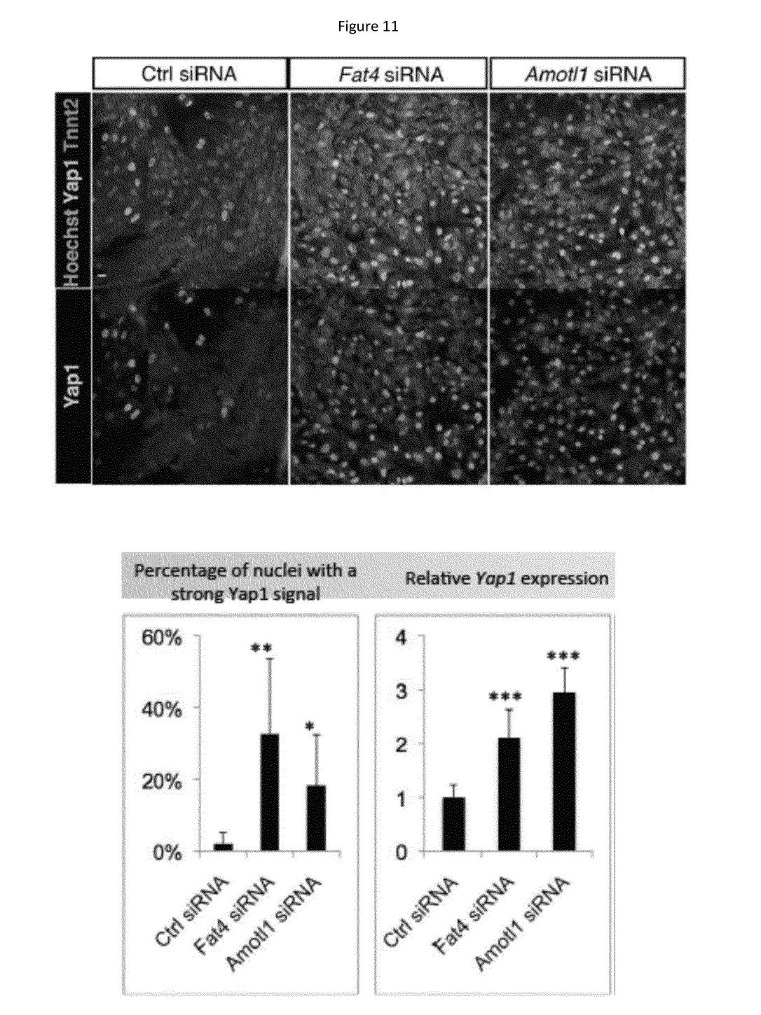

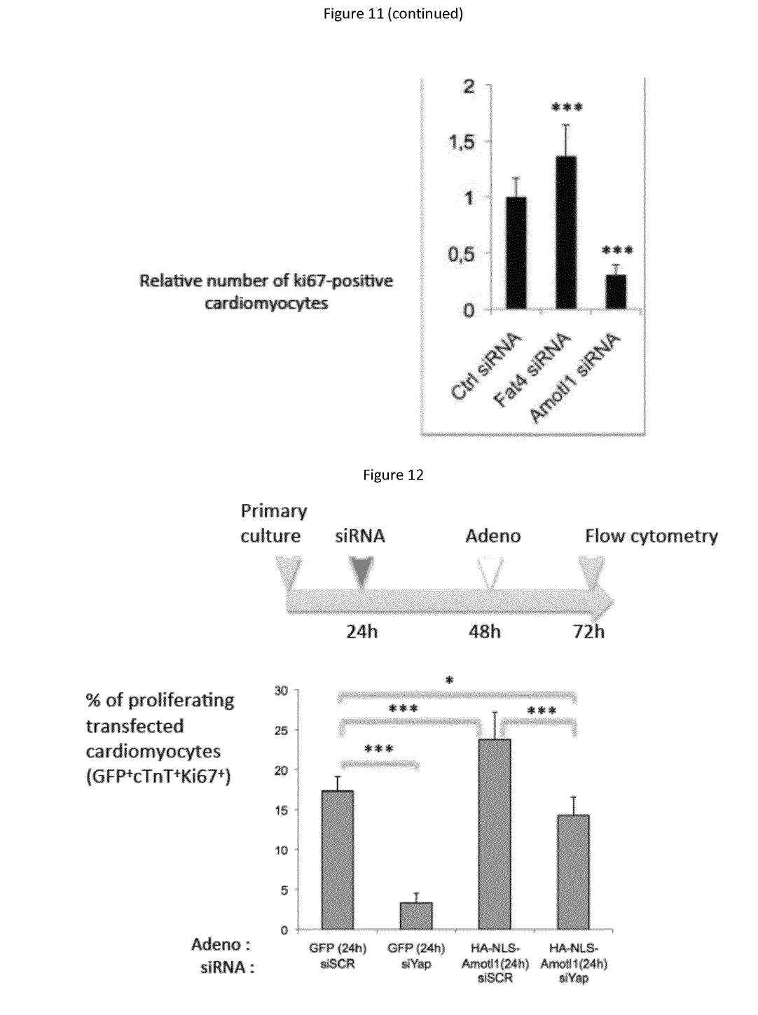

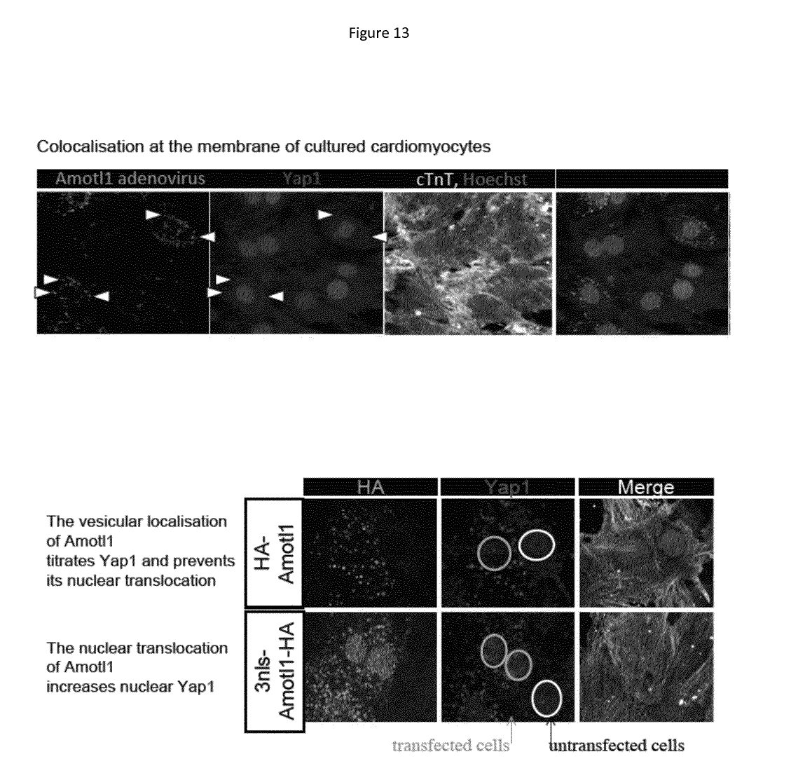

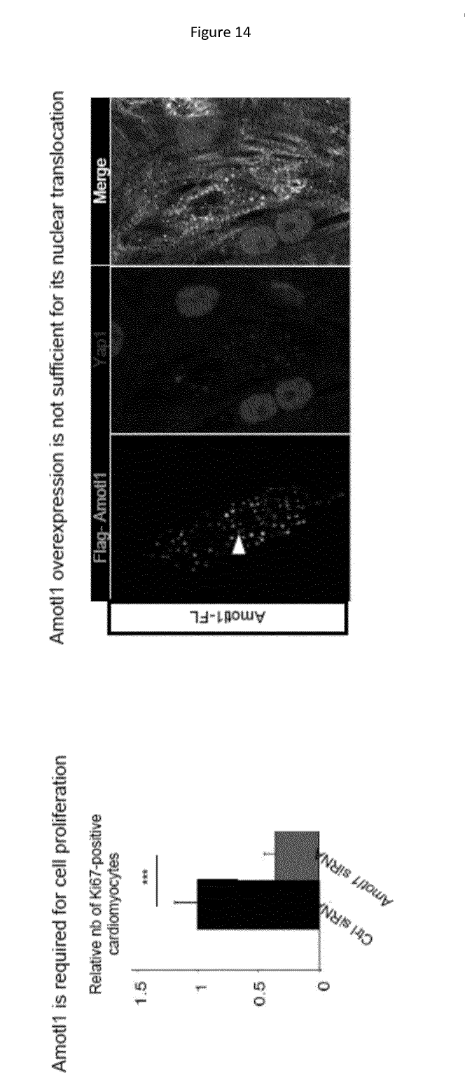

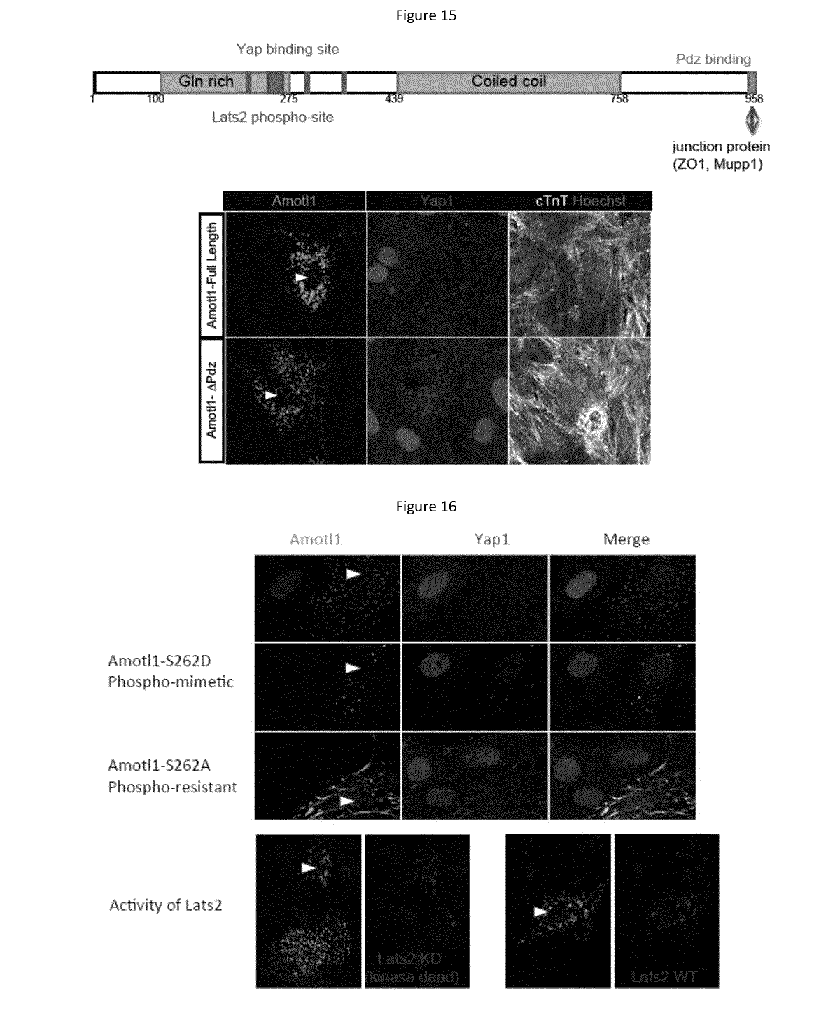

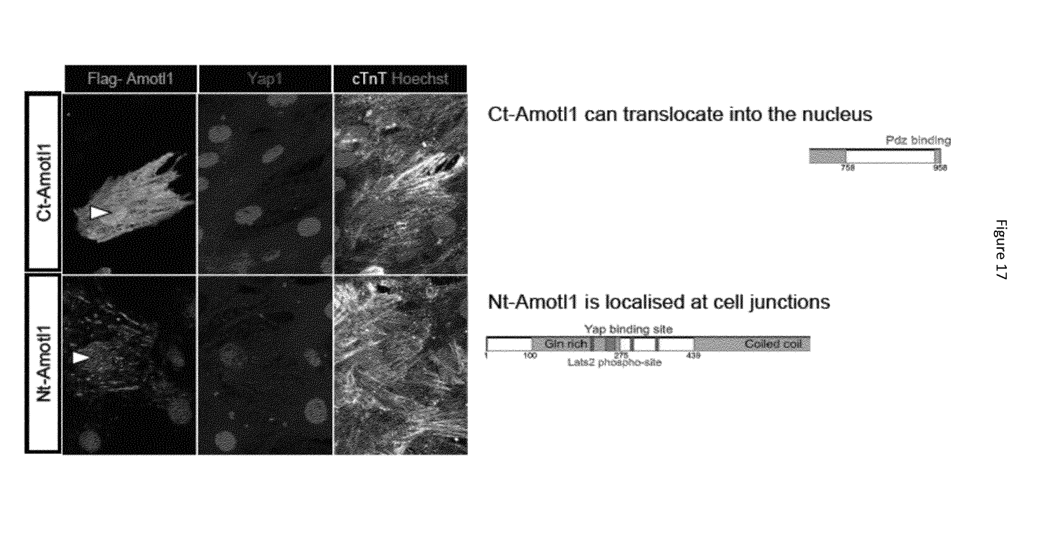

For quantitative data, when the number of observations n<5, the figures display all data points. For data shown as representative images, the number of replications of the experiments are: 1 experiment (FIG. 4b [n=1 Fat4+/+, 2 Fat4+/− and 2 Fat4−/− hearts], 4g [3 cultures], S4e [n=2 Fat4+/+ and 3 Fat4−/− hearts]), 2 experiments (FIG. 3b [n=3 Fat4+/+ and 3 Fat4−/− hearts], 4a [n=2 Fat4+/+ and 2 Fat4−/− hearts], 4c [n=3 Fat4+/+ hearts], S2b [n=4 Fat4+/+, 2 Fat4+/− and 2 Fat4−/− hearts]), 3 experiments (FIGS. 4i, 4j), 4 experiments (FIG. 8a), 50 litters (FIG. 1a), 3 litters (FIG. 7d), 1 litter at E14.5, 3 litters at E16.5, 2 litters at E18.5 (FIG. 9b).

2. Results

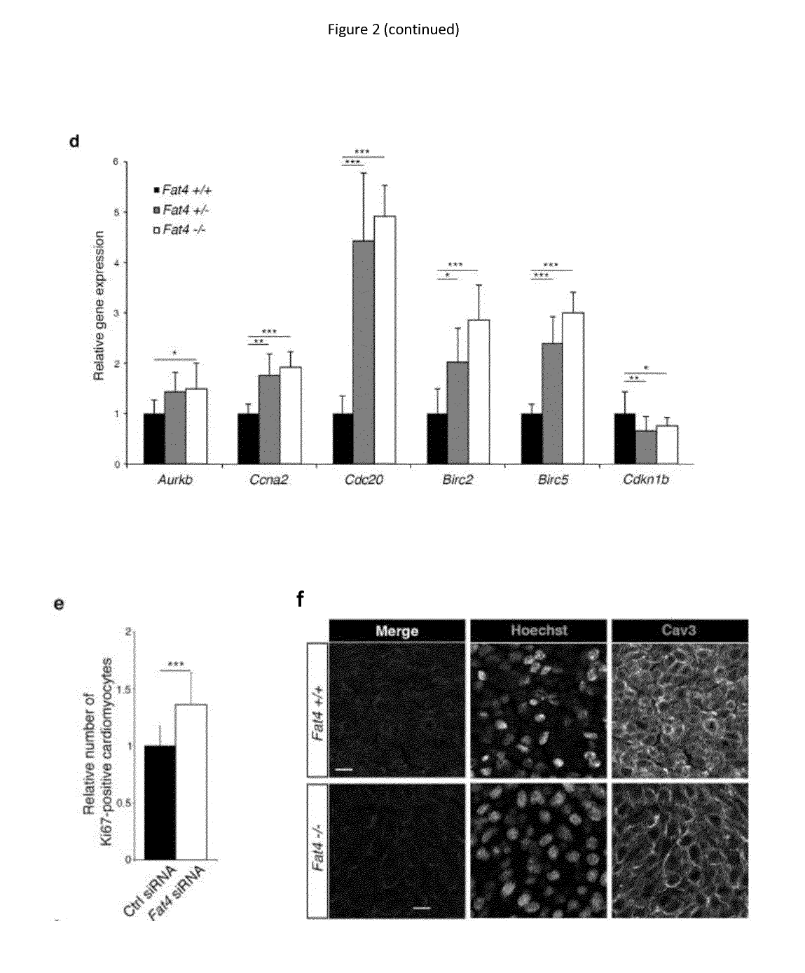

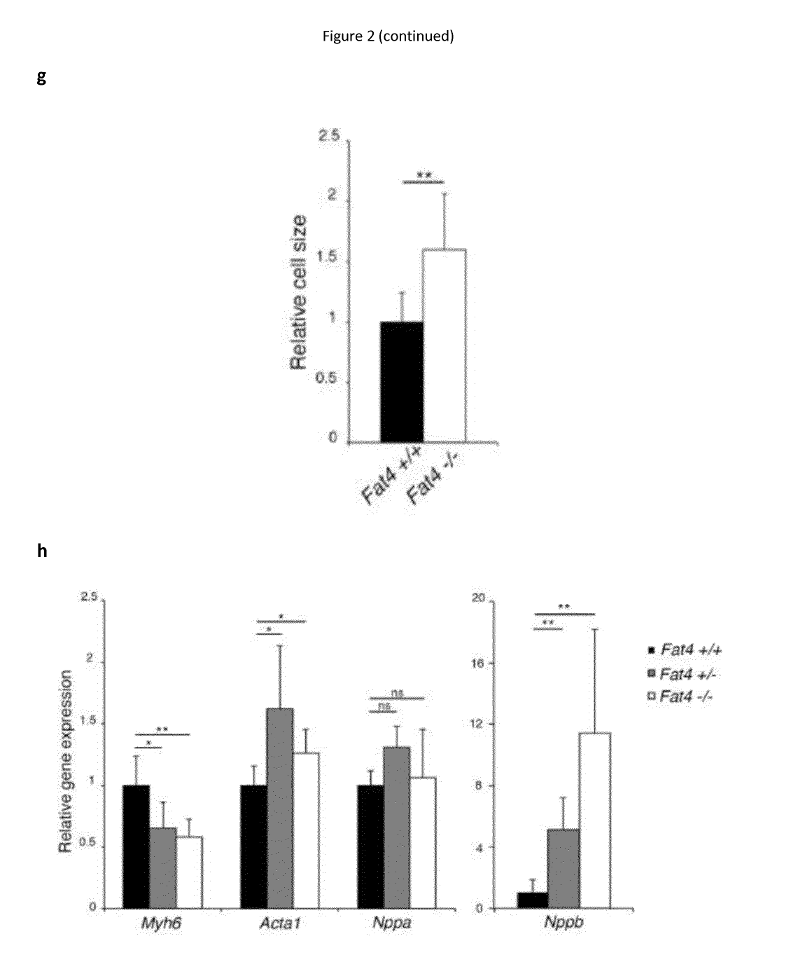

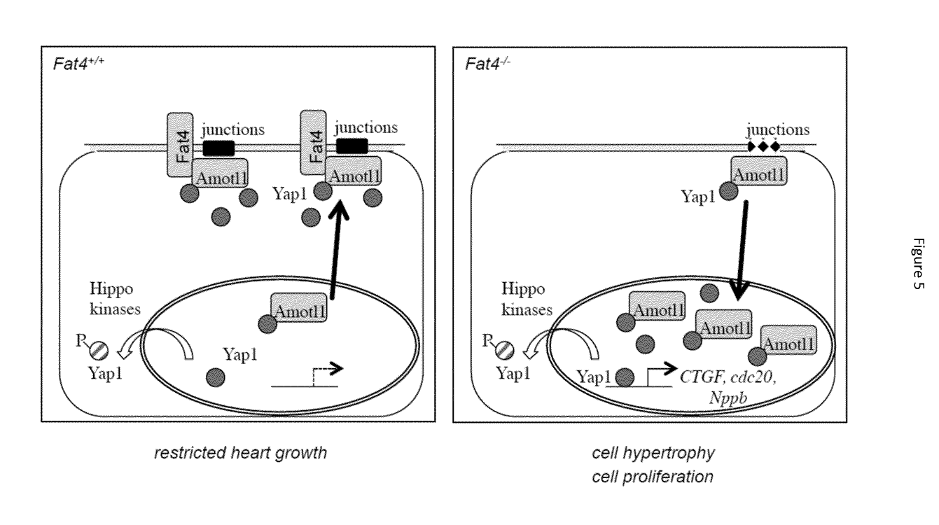

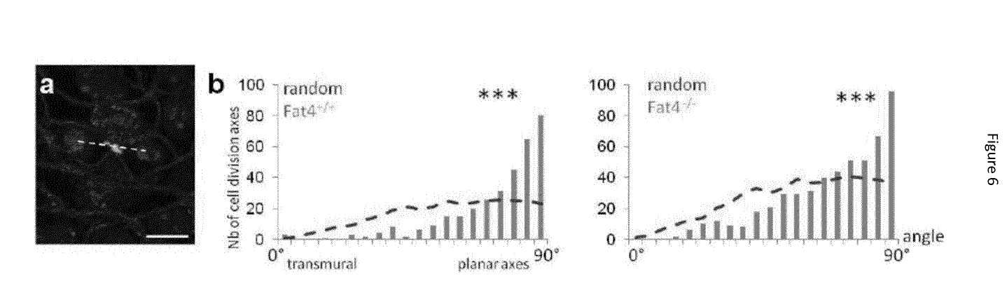

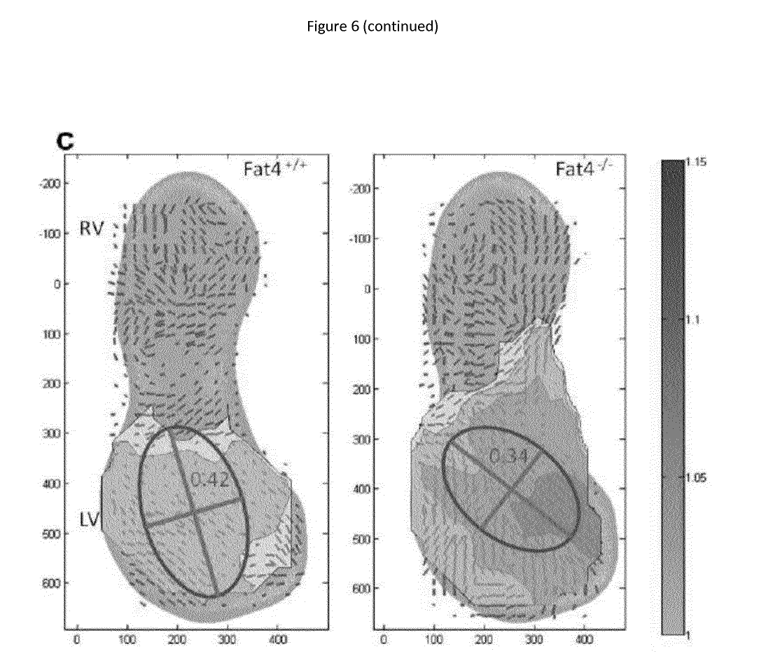

In the heart, the phenotype of Fat4^{-/-} mutants was assessed at birth. An abnormal flattened apex (FIG. 1a) and 1.5 fold thicker ventricular myocardium and interventricular septum (FIG. 1b-c) was observed, compared to control hearts. This excessive tissue thickness may result from defective orientation or excessive rate of growth. As it has been shown previously that clonal growth of the heart is oriented^{17,18}, the orientation of cell division was therefore analysed in Fat4^{-/-} mutants. Whereas in the mouse kidney orientation of cell division is disrupted when Fat4 is absent⁸, cell division was found still biased in the plane of the heart surface and still coordinated in the left ventricle of Fat4^{-/-} hearts as in controls (FIG. 6). It was then investigated whether the excessive growth of Fat4 mutant hearts was due to increased cell proliferation, and found that the number of cardiomyocytes positive for the mitotic marker, phosphorylated histone H3, was significantly higher in the absence of Fat4 (FIG. 2a-b), whereas the effect was milder in non-cardiomyocytes (FIG. 7a). Mutant cardiomyocytes undergo cytokinesis, as revealed by Aurkb staining (FIG. 7b). Conditional deletion of Fat4, in cardiac progenitors that express Mesp1, resulted in an increased percentage of cardiomyocytes, whereas their number of nuclei was unchanged (FIG. 2c, FIG. 7c). Conditional deletion of Fat4 in non-cardiomyocytes, using Wt1-Cre, did not change the number of mitotic cells (FIG. 7d-e), indicating that Fat4 is required in cardiomyocytes. Analysis of gene expression showed an increase for cell cycle (Ccna2, Cdc20), cytokinesis (Aurkb) and anti-apoptotic (Birc2/5) genes and a decrease of Cdkn1b transcripts implicated in cell cycle exit, in Fat4^{-/-} compared to control hearts (FIG. 2d).

Heterozygotes also show transcript upregulation, although they do not have a detectable heart phenotype, indicating compensation at the level of the proliferation gene network dependent on Fat4 dosage.

In primary cultures, knock-down of Fat4 (FIG. 7f-j) significantly enhanced the number of proliferating Ki67-positive and replicating EdU-positive cardiomyocytes (FIG. 2e and FIG. 7k-l). It was next examined whether Fat4 also affects cell size. By measuring the cross sectional area of cardiomyocytes, a significant increase of cell size was found in Fat4^{-/-} compared to control hearts (FIG. 2f-g). In agreement with a hypertrophic phenotype, RT-qPCR revealed a de-regulation of classical markers of heart hypertrophyl⁹, corresponding to the activation, in Fat4^{-/-} mutant hearts, of genes normally expressed at fetal stages (Acta1), whereas genes normally expressed at adult stages (Myh6) are down-regulated (FIG. 2h). The early marker of heart hypertrophy, Nppb²⁰, was strikingly increased (11 fold) in Fat4^{-/-} mutant hearts, whereas the marker of wall stress, Nppa, was not. These data show that Fat4 is required to restrict heart growth at birth, with an effect on the proliferation and hypertrophy of cardiomyocytes.

Since misexpression of genes (FIG. 2d) previously shown to be targets of the Hippo pathway in the control of cardiomyocyte proliferation^{1,3} was observed, it was examined whether Hippo signalling was impaired in Fat4 mutant hearts. The classical targets of Yap1, CTGF and Cyr61, were significantly overexpressed (FIG. 3a), showing that the transcriptional activity of Hippo effectors is increased in Fat4^{-/-} mutant hearts. Consistent with this, Yap1 was relocalised to the nucleus of cells in which Fat4 was down-regulated, both in vivo (FIG. 3b) and in primary cell cultures (FIG. 3c-d, FIG. 8a), and reduced in the nucleus of cells in which Fat4 was overexpressed (FIG. 3e-f). Knock-down of Yap1 transcripts by RNA interference (FIG. 7f-j) rescued the increased cell proliferation observed when Fat4 is down-regulated (FIG. 3g). Deletion of one copy of Yap1 similarly rescued the excessive number of mitotic cells in Mesp1-Cre conditional Fat4 mutant hearts, as well as the percentage of cardiomyocytes (FIG. 3h-i). These rescue experiments are consistent with the proposition that Fat4 acts upstream of Yap1. To examine whether canonical Hippo signalling was activated, as in the fly model, the phosphorylation of Yap1 at the Ser¹²⁷ target site of Hippo kinases was analysed. Neither in vivo, nor in primary cell cultures (FIG. 3j-k, FIG. 8b-f), could be detected a significant change in the ratio of phosphorylated Yap1, over total Yap1, when Fat4 was down-regulated. The phosphorylation of the Hippo kinases Lats and Mst was also unaffected when Fat4 is absent (FIG. 8g-j). A phospho-resistant form of Yap1 was also relocalised to the nucleus when Fat4 was down-regulated (FIG. 3l-m), further supporting the conclusion that the nuclear translocation of Yap1 downstream of Fat4 is not mediated by a change of phosphorylation. The phenotype of Fat4 mutants differs from that resulting from impairment of the canonical Hippo pathway. The onset of excessive myocardial growth in Yap1 gain-of-function mutants or in Hippo kinase-deficient hearts¹ is already seen at embryonic stages (E10.5-E11.5). In contrast, the phenotype of Fat4^{-/-} hearts is detected much later, from E18.5, although Fat4 is expressed throughout heart development (FIG. 8a-c). The Wnt pathway, which was previously shown to interact with the canonical Hippo pathway, was not found activated in Fat4^{-/-} mutants (FIG. 8d-e). These observations show that Fat4 is a later modulator of the Hippo pathway and suggest that a mechanism other than that of phosphorylation by the Hippo kinases, regulates the nuclear localisation of Yap1, downstream of Fat4.

Hippo signalling is modulated by cell junction proteins²¹. When cardiomyocytes were labelled with junction markers, abnormal cell junctions were observed in Fat4^{-/-} hearts. N-cadherin (Cadh2) or Plakophilin2 (Pkp2) staining were broader and less focalised than in control hearts (FIG. 4a). By electron microscopy, the electron dense desmosomal material was more diffuse and no gap junctions were detected in cardiomyocytes of Fat4^{-/-} hearts (FIG. 4b). In agreement with abnormal cell junctions, cardiomyocytes had a rounder shape in Mesp1-Cre conditional Fat4 mutant hearts (FIG. 8f). These observations suggest that a junctional protein may be involved in the effect of the atypical cadherin Fat4 on Hippo effectors. It was focussed on the adaptor protein, Angiomotin, which had been shown to interact with Yap1 and to co-translocate to the nucleus, where the complex modulates transcription²². This co-translocation can occur independently of Yap1 phosphorylation²³. In the heart, it was observed that Angiomotin-like 1 (Amotl1), another member of the Angiomotin family, is predominantly expressed (FIG. 8g). It is present near the membrane of cardiomyocytes, in clusters where Yap1 is also detected, between the z-lines of the sarcomere (FIG. 4c). Strikingly, Amotl1 was relocalised to the nucleus when Fat4 was absent (FIG. 4d-e). When it was interfered with Amotl1 (FIG. 7f-j) as well as Fat4 expression, the increased cell proliferation observed when Fat4 expression alone is down-regulated was reversed (FIG. 4f). Interference with Amotl1 expression alone shows that it is required for the proliferation of cardiomyocytes. On the contrary, forcing Amotl1 to translocate to the nucleus, by addition of a nuclear localisation signal, resulted in co-accumulation of Yap1 in the nucleus and stimulation of cardiomyocyte proliferation (FIG. 4g-h). Interference with Yap1 expression shows that the proliferative effect of nuclear Amotl1 is dependent on Yap1. Both Fat4 and Amotl1 are known to interact with the scaffold multi-PDZ domain protein, Mpdz, also known as Mupp1^{24,25}. It is now shown, in transfected HEK293 cells, that Amotl1 interacts physically with Fat4, as well as Yap1 (FIG. 4i), and that the three proteins co-localise in transfected cardiac cells (FIG. 4j). This is consistent with Amotl1 release from cell junctions when Fat4 is absent. These results identify Amotl1 as a modulator of the Hippo pathway in cardiomyocytes, and show that it is prevented from entering the nucleus by sequestration in a Fat4 complex, thus restricting Yap1 mediated tissue growth.

These observations of Yap1 activity in Fat4 mutants, as well as the suppression of Fat4 dependent hyperproliferation by reduced Yap1 or Amotl1 expression, argue that Fat4 is an upstream regulator of Yap1 in the mouse heart, and that it triggers a non-canonical modulation of Hippo signalling. This pathway probably implicates a non-phosphorylated form of Yap1 bound to Amotl1 in the cytoplasm. When Amotl1 is not sequestered at cell junctions with Fat4, it was shown that it is an intermediate, that bypasses the Hippo kinases, to regulate the nuclear translocation of Yap1. Amotl1 may also directly contribute to the transcriptional activation of target genes, by analogy with Amot in the liver²². It remains to be seen whether Tead, a transcription factor that interacts with Yap1, is implicated in this context. This model is shown in FIG. 5. Amotl1 has no homologue in flies, which explains why the intracellular domain of Fat4 cannot rescue the growth phenotype of fat mutant flies¹². The function of mouse Amotl1 is similar to that of Drosophila Expanded, a FERM-domain protein which requires Fat for its localisation at the membrane⁵ and which can directly sequester Yorkie out of the nucleus, independently of canonical Hippo signalling²⁶. The mammalian homologue of Expanded, Frmd6, has lost the C-terminal domain of interaction with Hippo effectors, which supports an evolutionary switch in the regulation of Hippo signalling by Fat¹⁶. Although Fat signalling is implemented differentially between mouse and fly, the function of this cadherin is well conserved, with a dual effect on tissue polarity⁸ and also, as the present inventors show, on tissue growth. The effect of Fat4 depends on the cellular context. In the heart, it was shown that Fat4 regulates tissue growth, rather than polarity. This has also been observed in the cortex¹¹, whereas in other organs, such as the kidney or the cochlea, Fat4 is a regulator of tissue polarity^{8,9}.

These findings on Fat4 mutants uncover a mechanism that restricts heart growth at birth. Central to this mechanism is the adaptor protein Amotl1, which can shuttle from cell junctions to the nucleus, transporting the transcription co-factor Yap1. Whereas the Hippo pathway was shown to be required at embryonic stages of heart development^{1,2}, Fat4 is a later modulator exerting its role at birth. It remains to be established how the Fat4/Amotl1 dependent pathway is activated and what is its relative importance to regulate Yap1, in comparison with canonical Hippo signalling. Canonical Hippo signalling is also modulated by cell junctions in cardiomyocytes, where remodeling of the intercalated discs activates Hippo signalling, with pathological consequences leading to arrhythmogenic cardiomyopathy²⁷. Fat4 mutants display hypertrophy, in addition to increased cell proliferation. Although hypertrophy can potentially be induced by Yap1^{4,28}, other studies^{2,3} would suggest that this is an indirect effect. Due to its positive effect on cardiomyocyte proliferation, Hippo signalling has been shown to be important for prolonging the regenerative potential of the mouse heart^{14,15}, which normally ceases during the first week after birth²⁹. However, Yap1 is less efficient in promoting cardiomyocyte proliferation at postnatal stages than it is during development, which suggests that other factors block Yap1 activity at later stages. It was now identified upstream regulators of Yap1 activity in the heart and it can be anticipated that manipulating the Fat4 pathway will facilitate the reactivation of cardiomyocyte proliferation induced by phospho-resistant Yap1¹⁵ or Hippo kinase deficiency¹⁴. This has major therapeutic implications for the repair of the failing human heart.

### REFERENCES

1. Heallen, T. et al. Hippo pathway inhibits Wnt signaling to restrain cardiomyocyte proliferation and heart size. Science 332, 458-461 (2011).
2. Xin, M. et al. Regulation of insulin-like growth factor signaling by Yap governs cardiomyocyte proliferation and embryonic heart size. Sci Signal 4, ra70 (2011).
3. von Gise, A. et al. YAP1, the nuclear target of Hippo signaling, stimulates heart growth through cardiomyocyte proliferation but not hypertrophy. Proc Natl Acad Sci USA 109, 2394-2399 (2012).
4. Del Re, D. P. et al. Yes-associated protein isoform 1 (Yap1) promotes cardiomyocyte survival and growth to protect against myocardial ischemic injury. J Biol Chem 288, 3977-3988 (2013).
5. Silva, E., Tsatskis, Y., Gardano, L., Tapon, N. & McNeill, H. The tumor-suppressor gene fat controls tissue growth upstream of expanded in the hippo signaling pathway. Curr Biol 16, 2081-2089 (2006).
6. Fanto, M. et al. The tumor-suppressor and cell adhesion molecule Fat controls planar polarity via physical interactions with Atrophin, a transcriptional co-repressor. Development 130, 763-774 (2003).
7. Ma, D., Yang, C. H., McNeill, H., Simon, M. A. & Axelrod, J. D. Fidelity in planar cell polarity signalling. Nature 421, 543-547 (2003).
8. Saburi, S. et al. Loss of Fat4 disrupts PCP signaling and oriented cell division and leads to cystic kidney disease. Nat Genet 40, 1010-1015 (2008).
9. Mao, Y. et al. Characterization of a Dchs1 mutant mouse reveals requirements for Dchs1-Fat4 signaling during mammalian development. Development 138, 947-957 (2011).
10. Bossuyt, W. et al. An evolutionary shift in the regulation of the Hippo pathway between mice and flies. Oncogene (2013).
11. Cappello, S. et al. Mutations in genes encoding the cadherin receptor-ligand pair DCHS1 and FAT4 disrupt cerebral cortical development. Nat Genet 45, 1300-1308 (2013).
12. Pan, G. et al. Signal transduction by the Fat cytoplasmic domain. Development 140, 831-842 (2013).
13. Li, F., Wang, X., Capasso, J. M. & Gerdes, A. M. Rapid transition of cardiac myocytes from hyperplasia to hypertrophy during postnatal development. J Mol Cell Cardiol 28, 1737-1746 (1996).
14. Heallen, T. et al. Hippo signaling impedes adult heart regeneration. Development 140, 4683-4690 (2013).
15. Xin, M. et al. Hippo pathway effector Yap promotes cardiac regeneration. Proc Natl Acad Sci USA110, 13839-13844 (2013).
16. Huang, J., Wu, S., Barrera, J., Matthews, K. & Pan, D. The Hippo signaling pathway coordinately regulates cell proliferation and apoptosis by inactivating Yorkie, the Drosophila Homolog of YAP. Cell 122, 421-434 (2005).
17. Meilhac, S. M., Esner, M., Kerszberg, M., Moss, J. E. & Buckingham, M. E. Oriented clonal cell growth in the developing mouse myocardium underlies cardiac morphogenesis. J Cell Biol 164, 97-109 (2004).
18. Le Garrec, J. F. et al. Quantitative analysis of polarity in 3D reveals local cell coordination in the embryonic mouse heart. Development 140, 395-404 (2013).
19. Izumo, S., Nadal-Ginard, B. & Mandavi, V. Protooncogene induction and reprogramming of cardiac gene expression produced by pressure overload. Proc Natl Acad Sci USA 85, 339-343 (1988).
20. Nakagawa, O. et al. Rapid transcriptional activation and early mRNA turnover of brain natriuretic peptide in cardiocyte hypertrophy. Evidence for brain natriuretic peptide as an “emergency” cardiac hormone against ventricular overload. J Clin Invest 96, 1280-1287 (1995).
21. Enderle, L. & McNeill, H. Hippo gains weight: added insights and complexity to pathway control. Sci Signal 6, rel (2013).
22. Yi, C. et al. The p130 isoform of angiomotin is required for Yap-mediated hepatic epithelial cell proliferation and tumorigenesis. Sci Signal 6, ra77 (2013).
23. Wang, W., Huang, J. & Chen, J. Angiomotin-like proteins associate with and negatively regulate YAP1. J Biol Chem 286, 4364-4370 (2011).
24. Sugihara-Mizuno, Y. et al. Molecular characterization of angiomotin/JEAP family proteins: interaction with MUPP1/Patj and their endogenous properties. Genes Cells 12, 473-486 (2007).
25. Ishiuchi, T., Misaki, K., Yonemura, S., Takeichi, M. & Tanoue, T. Mammalian Fat and Dachsous cadherins regulate apical membrane organization in the embryonic cerebral cortex. J Cell Biol 185, 959-967 (2009).
26. Badouel, C. et al. The FERM-domain protein Expanded regulates Hippo pathway activity via direct interactions with the transcriptional activator Yorkie. Dev Cell 16, 411-420 (2009).
27. Chen, S. N. et al. The hippo pathway is activated and is a causal mechanism for adipogenesis in arrhythmogenic cardiomyopathy. Circ Res 114, 454-468 (2014).
28. Wang, P. et al. The alteration of Hippo/YAP signaling in the development of hypertrophic cardiomyopathy. Basic Res Cardiol 109, 435 (2014).
29. Porrello, E. R. et al. Transient regenerative potential of the neonatal mouse heart. Science 331, 1078-1080 (2011).
30. Saga, Y. et al. MesP1 is expressed in the heart precursor cells and required for the formation of a single heart tube. Development 126, 3437-3447 (1999).
31. Wessels, A. et al. Epicardially derived fibroblasts preferentially contribute to the parietal leaflets of the atrioventricular valves in the murine heart. Dev Biol 366, 111-124 (2012).
32. Reginensi, A. et al. Yap- and Cdc42-dependent nephrogenesis and morphogenesis during mouse kidney development. PLoS Genet 9, e1003380 (2013).
33. de Chaumont, F. et al. Icy: an open bioimage informatics platform for extended reproducible research. Nature methods 9, 690-696 (2012).
34. Diguet, N. et al. Muscle creatine kinase deficiency triggers both actin depolymerization and desmin disorganization by advanced glycation end products in dilated cardiomyopathy. J Biol Chem 286, 35007-35019 (2011).
35. Megason, S. G. & McMahon, A. P. A mitogen gradient of dorsal midline Wnts organizes growth in the CNS. Development 129, 2087-2098 (2002).
36. Relaix, F. et al. Pax3 and Pax7 have distinct and overlapping functions in adult muscle progenitor cells. J Cell Biol 172, 91-102 (2006).
37. Pei, Z., Bai, Y. & Schmitt, A. P. PIV5 M protein interaction with host protein angiomotin-like 1. Virology 397, 155-166 (2010).
38. Dufour, A., Thibeaux, R., Labruyere, E., Guillen, N. & Olivo-Marin, J. C. 3-D Active Meshes: Fast Discrete Deformable Models for Cell Tracking in 3-D Time-Lapse Microscopy. IEEE Transactions on Image Processing 20, 1925-1937 (2011).
39. Charon, N. & Trouve, A. The varifold representation of non-oriented shapes for diffeomorphic registration. SIAM Journal on Imaging Sciences 6, 2547-2580 (2013).
40. Pop, S. et al. Extracting 3D cell parameters from dense tissue environments: application to the development of the mouse heart. Bioinformatics 29, 772-779 (2013).
41. Mollova, M. et al. Cardiomyocyte proliferation contributes to heart growth in young humans. Proc Natl Acad Sci USA 110, 1446-1451 (2013).
42. Moak J P1, Kaski J P. Hypertrophic cardiomyopathy in children. Heart. 2012 July; 98(14):1044-54.
43. Tariq M1, Ware S M1. Importance of genetic evaluation and testing in pediatric cardiomyopathy. World J Cardiol. 2014 Nov. 26; 6(11):1156-65
44. Sudol M1, Shields D C, Farooq A. Structures of YAP protein domains reveal promising targets for development of new cancer drugs. Semin Cell Dev Biol. 2012 September; 23(7):827-33.

1. A method for preventing or treating cardiac hypertrophy by reducing heart growth in a mammal, comprising down-regulating the Amotl1 and/or Yap1 Fat4-dependent activator(s) of the Hippo pathway, or up-regulating Fat4 in said mammal.

2. The method of claim 1, wherein said down-regulation is carried out by administering an effective amount of a siRNA inhibiting specifically Yap1 or Amotl1 expression.

3. The method of claim 1, wherein said down-regulation is carried out by administering an effective amount of verteporfin.

4. A method for diagnosing cardiac hypertrophy in a mammal, comprising analyzing the expression level of Amotl1 or detecting inactivating mutations in Amotl1 polypeptide sequence in a tissue sample from said mammal.

5. The method of claim 4, wherein, if Amotl1 expression level is enhanced as compared with a reference value, or if the Amotl1 polypeptide contains at least one inactivating mutation, then said mammal is suffering from or will develop cardiac hypertrophy.

6. The method of claim 5, further comprising the step of treating said mammal by upregulating Fat4 or by down-regulating Yap1 and/or Amotl1, or by increasing the nuclear localisation of Yap1 and/or Amotl1 or by reducing the sequestration of Yap1 and/or Amotl1 out of the nucleus.

7. The method of claim 6, wherein said down-regulation is carried out by administering an effective amount of a siRNA inhibiting specifically Yap1 or Amotl1 expression.

8. The method of claim 6, wherein said Yap1 down-regulation is carried out by administering an effective amount of verteporfin.

9. A method to induce heart growth or cardiomyocyte proliferation in a mammal, comprising down-regulating Fat4 in cardiomyocytes of said mammal.

10. The method of claim 9, wherein said down-regulation is carried out by administering an effective amount of a siRNA inhibiting specifically Fat4 expression.

11. The method of any one of claims 1 to 10, wherein said mammal is human.

12. A screening method for identifying compounds that are useful for preventing and/or treating cardiac hypertrophy, said method comprising the following steps:
a) administering a candidate compound to a transgenic mammal being deficient for the Fat4 gene,
b) monitoring cardiac hypertrophy or regeneration in said transgenic mammal before and after step a), and
c) selecting the candidate compound if it induces the reduction of cardiac hypertrophy in said transgenic mammal.

13. The method of claim 12, wherein step b) involves the monitoring of Yap1 dependent genes, such as Aurkb, Ccna2, Birc2, Birc5, Cdkn1b, Lyh6, or Acta1.

14. The method of claim 12, wherein step b) comprises quantifying cardiomyocyte proliferation and/or shape.

15. The method of any one of claims 12 to 14, wherein said transgenic mammal is a Knock-out Fat4^{-/-} mouse or Fat4^{-floxflox} mouse.

16. A screening method for identifying compounds that are useful for preventing and/or treating cardiac hypertrophy, said method comprising the following steps:
a) contacting a candidate compound to at least one cell which is deficient for the Fat4 gene,
b) monitoring the proliferation and/or the size of said at least one cell before and after step a), and
c) selecting the candidate compound if it is able to reduce the proliferation and/or the size of said at least one cell.

17. The method of claim 16, wherein said at least one cell is a cardiomyocyte.

18. The method of claim 16 or 17, wherein said at least one cell is a Fat4^{-/-} mouse cardiomyocyte.

19. A screening method for identifying compounds that are useful for preventing and/or treating cardiac hypertrophy, said method comprising the following steps:
a) contacting a candidate compound with at least one cardiomyocyte cell,
b) monitoring the expression level of Fat4 in said cell before and after step a), and
c) selecting the candidate compound if contacting said cell with said candidate compound enhances Fat4 expression.

20. A screening method for identifying compounds that are useful for preventing and/or treating cardiac hypertrophy, said method comprising the following steps:
a) contacting a candidate compound with at least one cardiomyocyte cell,
b) monitoring the expression level or the subcellular localisation of Yap1 and/or Amotl1 in said cell before and after step a), and
c) selecting the candidate compound if contacting said cell with said candidate compound reduces Yap1 and/or Amotl1 expression or increases their sequestration out of the nucleus.

21. A screening method for identifying compounds that are useful for increasing heart size or inducing heart regeneration, said method comprising the following steps:
a) contacting a candidate compound with at least one cardiomyocyte cell,
b) monitoring the expression level of Fat4 in said cell before and after step a), and
c) selecting the candidate compound if contacting said cell with said candidate compound reduces Fat4 expression.

22. A screening method for identifying compounds that are useful for increasing heart size, said method comprising the following steps:
a) contacting a candidate compound with at least one cardiomyocyte cell,
b) monitoring the expression level or the subcellular localisation of Yap1 and/or Amotl1 in said cell before and after step a), and
c) selecting the candidate compound if contacting said cell with said candidate compound enhances Yap1 or Amotl1 expression or increases their nuclear translocation or reduces their sequestration out of the nucleus.


SEQUENCE LISTING (27 sequences)

SEQ ID NO: 1 - DNA, 16109 bp, mus musculus
  misc_feature: mouse Fat4 cDNA
ggttcctggaaactaggaccatgaacttggccgcaaaccgggctcctggccgcaggcggctcccgctaccttctccatctctgtgccagctcctgagagtctgggggctgctatcactgctccccggctccgcccgagtccaagcggccgaacagcgccaggtgttccaggtgatggaagagcagcccccagggactctggtgggcaccatcccaacgcgtcctggctttacctatcgtctcagtgaaagccacgccctgttcgcgattaacagcagcaccggagcgctgtacaccaccgctaccatcgaccgcgagagcctgcccagcgatgtggtcaacctggtggtcctttccagctcccccacataccctaccgaggtgcgagtgctggtgcgtgacctcaatgacaacgccccagtcttcccagacccttcgatcgtggtcaccttcaaggaggacagtggtagcgggcgtcaggtcattctggacaccgccacggactctgacatcggctccaacggtgtggaccaccactcctaccgcatcgtcagcggcaacgaggcgggccgctttcggctggacatcaccttgaaccccagtggcgagggagcgttcctgcacctggtatccaagggcgggctggaccgcgaagtcacacctcagtaccagctcctggtggaggtggaggacaagggtgagccaaagcggcggggttaccttcaggtcaacgtaactgtacaggacattaacgacaatcccccggtgtttggcagttcccattaccaggcgggggtgcctgaagacgcagttgtgggctccagtgtcctgcaggtggcggcggcggacgcagacgagggcaccaatgcagacatccgttaccggctgcaagatgagggaacccccttccagatggacccagagacggggctcatcaccgtgagagagccgctagatttcgaggcccggcgacagtattcgctaacagttcaggccacggaccggggcgtgccctctctcaccgggcgcgccgaagcgtttatccagttgttagatgtcaatgacaacgacccggtagtgaagtttcgctacttccctgccacctcgcgctatgcttcggtggatgagaacgcgcaggtgggtacagtggtggctctgctcaccgtgactgacgctgactccccagcagccaacggaaacatttcagtgcagattctcgggggcaatgagcagcgccactttgaggtgcagaggagcaaggtgcccaacctgagcctcatcaaggtggccagcgccctggaccgggagcgcatcccttcatacaacctcacagtctcggtctcggataactccggggcgccccctaccgccgaggtccaggctcgttcgtctgtggccagcttggtgatttttgtcaatgacatcaatgatcaccctccggtctttgagcagcaagtatacagagtgaacctgagcgaggaggtgcctccggggagctacgtgagtggggtgtcggccaccgatggcgactctggtctgaatgctaacctgcgttacagcattgtctccggaaatgggttgggctggtttcacattagtgagcacagtggactggtgactaccagtgctgccgggggccttgaccgtgaacttgcttcccagattgtactaaatattagtgcccgagaccagggggtccaccccaaggtatcctatgcccagcttgtggtaactgtcctggatgtcaatgatgaaaagccggtgtttagccagcctgaaggttatgaggtgtctgtggttgaaaatgctccaactgggacagagttgctggttctcggggcaacagacagggatctgggtgacaatgggacagtgcgcttctccctgcaagaggcagagaatgaccaaaggctcttccgcctggatccggtatctgggaggctgagcactgcctcctccctggacagagaggaacaagccttctactgtctatcgattctagccacagacctgggttcccctccccagtcgtccacagctcaggtaaatgtcagtcttctggatataaacgataacagccctgtgttctacccagtccaatactttgctcatatccaggaaaatgagcctggaggtagctatgtcaccacagtgtctgctaccgacccagacatgggtcccaacggaactgttaagtacagtatatcagctggggacaggtctcggtttcagattcatgcgaagagtggggtcatttctacaaaaatggccctagatagagaagagaaaacagcataccagctgcaagtagtggccacagatgggggcaatctacagtctcccaaccaggccatcgtaactgtcacagtgttagacacacaagacaacccacctgtgttcagccaggctgcctacagctttgtggtctttgagaacgtggctctggggtatcatgtgggcagtgtttctgcaactacaatggacctcaatgccaacatcagttatctcatcactaccggggaccagcgagggatgtttgctatgaatcctgtcacagggcagctgaccacagcaagtgtcattgacagggaagaacagtccttttatcagctgaaaatagtggccagtgggggagccgtcacgggagacactgtggttaacatcacagtgaaagatttgaacgacaacgccccccacttcctccaggcagtggaaagcataaatgctgtggagaactggcaggcgggacacagcatcttccaggccaaagccgtggacccggatgagggtgtcaacggtagggtgctctatagtctgaagcagaaccccaagaacctgtttactattaatgagcagaatggtaacattagtctgctgggggccctagatgtccacgctggctcataccaggtagaaatcgtggcctctgatatgggtgtccctcagctgtcctccagtatccttctgacagtttacgtgcatgatgtaaatgacaacccaccagtgtttgatcagatttcctacgaggtcacactctccgagtcagagcctgtgaattctcgattcttcaaggtacaagcttcagacaaagattctggagcaaatggggaaatagcatacacgatcacagatgggaacaacggggatgcctttggcatattcccagatggccagctgtatataaaaagtgaactggatcgtgaacttcaggacagatatgtgttactggttgttgcctctgaccgggcagtggagcctctaagtgctactgtgaatgtcactgtgcttttagaagatgtgaatgacaacaggcctttgtttaatagcaccaattacacattttacttcgaagaggagcagagagctgggtccttcgtaggcaaagtcagtgctgtggacaaagacttcgggccaaatggggaagtcaggtatgcttttgaggtgacacagccaaatttcgagttgcacgctgtcactggggagatcaccagcactcacaagtttgatagggagtccctcatgaggagaagggggacagcagtattcagcttcacagtgacagccatggatcggggcctccctcagcccctcaaggaccaggccactgtgcatgtctacatgaaggacatcaacgacaatgcccctaagtttctaaaagacttttaccaagctacagtatccgagacggcgaccaatctgacccaggtcttacgagtgtctgcctccgacgtggacgagggaagcaacggtctcattcattactctatcctcaaaggaaacgaagaaagacagtttgccatagacagcttctctggacaggtgactcttgtaggcaagctagactatgaagcaacctcagcctactctctcctcatccaggcagtggattcaggggccattcccctcaactcaacctgcaccctgagtatcgatattttggatgagaatgataacaccccctctttcccaaagtcaacactattcgtggatgttttggaaaacatgaggattggcgaacttgtatcctccgtcactgccactgactctgactcgggtgtcaatgctgacctacactacactattacagggtcaaacaaccatgggacttttagtattagccccaacactgggagtatctttctggccaaaaagttggactttgagacacagtctttatataaattaaacataacagcgaaagaccaggggcggcctcctcgatcgtccacgatgtcagtggtgatccaggtacgggacttcaatgacaacccccccagctttcctccgggagacatttttaagtcaatagtggagaacatcccccttggtacgtctgttatttccgtgacagcgcatgacccagatgcagatatcaacgggcagctgtcttacgccatcatccaacagatgccccgaggcaaccacttcagcatagacgaagtcaaaggaaccatctacaccagtgctgagatcgaccgggaatttgccaacctcttcgagttaacggtgaaagccaatgaccaagcagtgccgatagaaaccaggaggtacgctctgaaaaatgtgaccattttggtgaccgacctcaatgacaatgtcccaatgtttatatctcaaaatgcactggctgcagacccgtcagccatgattggttctgtcctgactaccatcatggctgcagacccggatgaaggtgccaatggcgaggtcgagtatgagatactcaatggggacacagacacctttactgtggatcgttacagtggggatcttcgagtggcttctgccttggtgccttcacagctcatctacaaccttatcgtatcagccacagacctgggcccagagcggaggaagtcaaccacagagctaactgtcattctccagggcctagatgggcctgttttcacacagaccaagtacataaccatcttgaaggaaggggaacccattggcaccaatgtcatctccatagaagcagccagccccagagggtcagaagctccagtggaatattacatcgtttctgttcgttgtgaagagaagactgtcggacgtctctttaccattggacggcagacgggtgtcattcaaactgcagccattttggaccgggagcagggtgcgtgtctctacctggtggatgtttatgccatagagaagtcctctgcctttcccaggacacagagagcagaggtagaaataacacttcaagatatcaatgacaacccaccggtgtttccaacagacaccctggatctcaccgtggaagagaacattggagatggctcaaagattatgcagctgactgccatggatgctgatgagggtgccaacgctctggtcacgtatgctctcattagtggagctgatgacagcttccgaattgatcctgagtctggagacctgatagccaccaagaggttggacagggagcacaggtccaagtactcattgctggttcgtgcagacgatggcctccagtcctcagacatgaggataaatatcaccatcagcgacgtgaatgaccacacccccagattctccaggcccgtgtactctttcgacatcccagaagacacaacccctggttccctggtggcagctattttagcaacagatgatgactctggtgtgaatggagagatcagctatgtggtggaggaggatgacggggatggcgtgttctttctgaatctggtcactggtgtcttcaacctgacccgagcactggactatgaaacacagcagtattacatcctcacagtccgtgcggaggatggtgggggtcagtccaccaccatccgagcttacttcaacattcttgatgtcaacgacaatccacctgtcttcagcatgagctcctacagcacatccttaatggaaaacctgcctctcggatccacggtccttgtgtttaacgtcaccgatgcagatgatggtgtcaactctcagctgtcctacagcattgcttcaggtgacagccttgggcagtttgcagtggataagcacggagtcctcaaaaccctcaaagctttggaccgggaaagccagtccttctacaacctggtcattcaggtccatgacttgccccagcctcctacctccaggttcaccagcacagcccaagtttctatcatcttgttagatgtcaatgataacccacctatgtttctttcccccaagttgacatacattccagaaaacacacccattgatactgttgtcttcaaggctcaagcgacagacccagacagtggccccaacagctatattgagtatactctcctgaacccttcaggaaacaagttcagcattgggaccattgatggtgaagtgcatcttacaggagagctggacagagaggaagtttccaattacagcctgacagtggtggccacagacaaagggcagccacctctgtcgtcctctactgaggttgtggttatggtccttgacatcaatgataacaaccctgtttttgctcaggctatgtaccgagtgcagattaaggagaacatactcactggaacagatataatccaagtgtctgcagcagacaatgatgaaggtaccaacggacaggttcgctacggcatcgtgggtggaaacacacatcaagagttcaggatcgactcagtgacaggggccatcacagtggctaagtctttggatagagagacgacccctgcttacactttaactgttcaggccacagatcggggcagtagccccagaacggactcctgcacagtcgccatcactttacttgacatgaatgattttgtccctgtatttgagttgtctccgtattctgtgaatgttcctgaaaatttagggaccctgcccagagccattcttcaggtggtggcaagagacgatgatcaaggtccaaatagccagctctcatatgttttgcttggtggaaatgaagacaatgcttttgtcctcacggccagtggcgagcttcgggtgacccagagtctggaccgggaagcaagagatcacttcgtcctggttgtcacagctgctgatgcaggatccccagccttgacgggaacaggaacaatcaacatcattgtagatgatatcaacgacaacgtccccacttttgccaataacatgtacttaacttcaattgccgaggatgcccggacggggacagatgtcctgttggtgaatgcctcggatgcagatgctgcggcaaatgctgtcatcagttacagtatcatcggtggaaactcgcagttcaccatcaacccatccacgggacagatcatcactagtgctttgctggacagagaaaccaaagataattacactttggtcgtggttgccagtgatgctgggtctccagagtctctttccagctctactagtgtgcttgtgaccatcactgatgttaatgacaacccaccccggttccaacatcacccatacgtcacacacatcccatctcctactcctccaggttcctttgtttttgctgtcacagtcaccgacgcggacataggatctaattctgaactccattactcactctcaggtagaaactctgaaaaatttcacattgaccccctgaggggggcaatcatggcagctgggccactaagtggagcttcggaggtgacgttctctgtccatgtgaaagatggtggctcatttccaaagacagattctactacagtgactgttaggtttgccaacaaggcagatttccccaaagtcagagccaaagaacaaacattcatgtttccagaaaaccagccagtgggcactctggtcaccactatcacagggtcctctttgagaggagaaactttgtcttactacatcgcaagtggaaacctgggcgacactttccaaattgatccactaacaggacaggtgtccatcagccagcctctggactttgagaagatacaaaaatacgttgtttggatcgaagctagagatggaggcttccctcccttctcttcttatgagaagcttgacatcacggtgctggatatcaatgacaatgctccaacatttgaagaagacccatttgtatctgagatcctggaaaacctatcccctcggaaaatccttaccgtctcagcaacggacaaggacagtggtcccaatggacaactagactatgagattgtgaatggcaaccaggagagcagtttcaccatcaatcatgctacaggtgaaatcagaagcattcggccactagacagagaaaaaatatcccactatgaactcactgtgaaatcttcagacaaagggtccccttctcagagcacctccgtaaaagtgatcatcagtattttagatgaaaacgataacgcacctaggttttcacagatattcagtgcctatgtttctgaaaactcccccttgggatacacagttacccgtgtaacaacttctgatgaagacattgggataaatgcaattagtagatattccatagtggacacaagtcttccatttactattaaccccaacacaggagatattgtcatcagtcgacctttaaacagagaagatacagacagatacaggatccgagtgtccgcacatgattctggctggactgtaagtaccgatgtcaccatatttgtgactgacatcaatgacaacactccaagatttagcagaccctcttattatttagattgccctgaacttcctgagcttggctccagagtgactcaagtgtctgccacagatcctgatgaggggtcaaatgggcaggtgttttatttcataaagtcgcagtcagagtatttcagaatcaatgccaccactggagagattttcaacaagcaggtgttaaaataccaaaatgtcagtggcttcagtaatgtgaacatcaacaggcacagtttcattgtgacagcttctgaccgaggcaacccctccttactcagcgagaccacagttaccatcaacacggtggatagcaacgacaacccacctcagtttctccaaaataaatactttaccccagttaccaaaaacgttaaagttggtactaagttaatcaaagtgactgcggtggatgacaaagactttggattaaactctgaagtggagtatttcgtgtcggatgggaaccacctagggaagtttaagttagacaacgatactgggtggatttcaatagcctcttccttggtttctgacctaaatcaaaacttcctgatcagagtcacagcaaaggacaaagggaaccccccactttcctcccaagcagtcgttcacatcacagtcactgaagagaactaccacacccctgaattttcccagaaccatataagtgccaccatccctgagagccacagcattgggtctgtggtcagaactgtctctgccagagatcgtgacaccgccatgaatggtctgatcagttacaacattatttcggggaatgaagagggcatctttgcaatcaattcctcaacaggtgtggtaaccctagccaaagcccttgattatgagatgagctcaaagcacgagatgaccattagtgccacagatggaggatgggttgcgaggactggctactgcagcctgactgtgagtgtgatcgatgtgaatgacaattccccagtgtttgttcctgatgaatttttcccaactgttatggagaacgccccaagtgggaccactgttatccacctaaacgcaacagacgctgactcgggagccaatgctgtgattgcgtacactgtgcagtcatcagacagtgacctgtttgttattgaccccaacatgggagtcatcaccactcaaggcttcttggattttgaaaccaagcagagctaccacctcacagtgaaagccttcaacgtccccgacgaggaaaagtgcagctttgccactgtggatatccagttgaaggggacgaacgagtatgtgcctcgctttgtttctaagctctactactttgaggtgtcggaagcggcttctagggggactgctgttggggaagtgtttgccagtgacagggacatgggtgctgatggagaagtacactatttgatttttggaaatagtaggaaaaagggcttccagatcaacaagatgacagggcagatctacgtgtctgggcttcttgatagagagaaagaagaaagggtgtctttgaaagtgttagccaagaactttggcaacattcgaggtgcagatatcgatgaagtcacagtaaatatcactgtgcttgatgctaatgacccgccagtgtttagtctgagcacctatagagtccagatcagtgaaggggtccctattgggacccacgtgacctttgtgagtgcctttgactcagattccatcccgagctggagcaggttctcatacttcattgggtccgggaatgaaaatggtgccttttccattaacccacagacagggcagatcacagttacctcagggctggaccgggaaagcctgcctgtttataatctcactgtcctggctgtagactcagggactccctctgccacaggaagtgcttccttggtagttaccctggaagacatcaatgacaatggcccagtgttgactgtcagtgaaggggaagttctagaaaacaaacgaccaggaactctagtgatgaccctgcagtccacagatcctgacctccctccaaatcaaggccccttcaattattacctgctgagcacaggccccgctactaattatttcagtctcagtaccgctggcgttctaagcacaacccgggagatcgacagagagcagattgcggacttctatttgtctgtggtcaccagggattctggggctcctcaaatgtcatccacaggaactgtgcacatcactgttctagatcaaaatgacaacccctcacagtcccgcacagtggagatatttgttaattattacggcaacttatttcctggtggcactctgggctctgtgaagccccaggacccggatgtgttagacagtttccactgctccctcacttcaggagtcaccagcctcttcagtatcccagcgggcagctgtgacctgagttcccagcccagatccacagatggcacctttgatctgactgttgtcagcagcgatggggttcacagcacagtcaccaacaatattcgagttttctttgcaggattctccaatgctacgatagataacagcatcttgcttcgtgttggtgttcccacagtcaaggacttcctgactaatcactaccttcactttctgcgcattgctagttcccagcttacgggcctgggcacggcagtgcagctgtatgctgcctatgaggagaacaacagaacgtttcttttagcagctgtgaaacgaaataataaccagtatgtgaaccccagtggtgtagccacgttcttcgaaagcatcaaagagatcctcctccgtcagagtggtgtcaaggtcgaatctgtggatcacgacccatgcattcatgggccgtgtcagaacggaggcagctgccttcgaagacttgccgtgggctctgcgctgaagattcaggagagtctgcctgtcatcattgtggccaacgagcctctgcagccttcccagtgcaagtgtgtgccaggctatgctggaagctggtgcgaggtggatattgatgaatgcctcccagccccttgccacaatgggggcacctgccacaacttagttgggggattttcatgcagctgcccagagggcttcactgggagagcctgtgagagagacatcaatgaatgtttgcccagcccttgcaagcatggggccgtctgtcaaaatttcccagggggattcaactgcgtgtgcaaaactggatacacaggaaaaatgtgtgaatcttcagtcaattactgtgaatgcaacccctgcttcaatggtggttcctgtcagagtggtgtggagtcctattactgtcactgtccattcggcgtctttggcaagcactgtgagctgaacagttatggatttgaggaactgtcatacatggagtttcccagcctggaccccaacaacaactatatttatgtcaagtttgccaccatcaaaagccatgctttattgctttataactatgacaaccagacgggagagagggcagagttcctggctctggagattgccgaagaaagactcagattctcatacaatctgggcagtgggacgtacaaactgaccaccatgaagaaggtgtcggatggacagttccacacagtgattgcacggcgagcaggcatggcagcttctttaactgtggactcctgctcagagaatcaagagccaggctattgcactgtcagtaatgtggcggtttcagacgactggactcttgatgttcagccaaaccgagtcactgttggaggaatcagatcactagagccaatccttcagaggagggggcatgtggagagccacgattttgtgggctgtgtaatggagtttgctgtcaatggacgacccctggagcccagccaggctctggcagcacaaggcattcttgaccagtgtcctagattggaaggcacgtgtgctcgcaacccctgccagcatggtggcacctgtgtggacttctggtcatggcagcagtgtcagtgcatggagggtctgactgggaagtactgtgaaaaatctgtcactccagacactgccttgtcactagaaggcaaaggacgcctggactaccacatgagccagagtgagaagcgggaatacttgctgacccagagcatacgggacaccacattggagccttttggggtcaatagcctggaagtaaaattcaggactcggagtgagaatggcattttgatccatatccaggaaagcagcaattataccacagtgaagattaagaatggcaaagtgcactttacatcggatgcaggtgttgctgggaaggtggagcgaatcatcccagaagcatacattgcagatggccattggcataccttccggatctcaaagaatggaagcatcactgtgctgtctgtagacaggatacacaacagagacatcgtccaccccactcaggactttggtggcattgaggtgctttccatgtctctgggaggaatcccccccaaccaagcccatcgagacactcagacaggttttaatggctgcattgcctcagtgctctatggtggagagagcctccccttcagtgggaagcacagcttggcgtccatctccaaaacagacccttcagtgaagatcggctgtcggggccctaacatctgtgccagcaacccctgctggggcgatcttctgtgtatcaaccagtggtatgcctacaaatgtgtccctcctggtgactgtgcatcccacccttgccagaatggaggtagctgtgaaccaggtctgctctccggctacacttgcagctgtccagagtcgcacacagggaggacctgtgagacggtggtggcctgcctcggtgttctctgtcctcagggcaaggtgtgcaaggctggaagtccagggggacacgtgtgtgtccagagtcagggtccagatgagatatccttacctctgtgggcagtgcctgcaattgtgggcagctgtgccacagccctagcgctgctggtcctcagtttgattctatgcaaccagtgcaggggaaagatgcccaaaaaccccaaagaggagaagaagccgaaggagaagaagaaaaagggcagtgagaatgtagcttttgatgacccagacaacatccctccctatggggatgacctagctgttaggaagcaacctgaggggaatcccaagccggacatcatagagcgagagaacccctatctaatcttcgatgagactgacatcccccacaactcagagaccatccctagtgccccgctggcttcccctgagcaggagattgagcactatgacatcgacaatgccagcagtatcgcgccctcagatgcagacatcatccagcactacaaacagttccgaagccacacgcctaagttttccatccagaggcacagtcctctgggcttcgccaggcagtcccctatgcccttgggagcaagcagtctgacctaccagccttcttcatatggccaagggttaagaaccagctcccttagccactcggcctgccccactcccaaccctctgtccaggcacagcccagctcctttctccaaaccatctgccttctacaggaacagcccagcccgggaactgcatcttcctctgagggatgggggtacgctggaaatgcatggggacccctgccagcctggcatgttcaactatgccaccaggctagggagaagaagtaagagccctcaggccatggcatctcatggatcgagaccagggagtcgcctcaaacagcccatagcacagatccctctagaatcttctcctcccgtgggactctccatagaggaggtagagaggctcaacacccctcgacccagaaaccccagcatctgcagtgcagaccatgggaggtcctcttctgaagaagactgtagaaggcccctgtccagaaccaggaacccagcagatggcatccctgccccagaatcgtcttctgacagtgactcccatgattctttcacgtgctcagaaatggaatacgacagggagaagcccgtggtctacacttccaggatgcccaagttatctcaagtcaacgaatccgatgcagatgacgaagataattatggagccagactgaaacccagaaggtaccatggacgcagggctgaggggggaccagtgggtacccctgcagcagcgtctggggctgcagacagcaccctgaagctggggcagcaggcaggcaacttcaactgggacaaccttttgaactggggcccaggctttggtcattatgtggatgtctttaaagatttggcctctctcccagaaaaagcagctggtaacgaagaaggtaaaagtggggcagctaaaccagccgccaaagatggggaggcagaacagtatgtgtgaggtgtatgtgtgggcaagaaacactcaaagcacggtgtggcttcccaccagcgggggtcacctttgtaaaacaggccagtatagaccggtggccagggacacatttaaagttaaccacaaagctgctcacatagactccaaacgactgactctttaatttaaatgtgctcagtggaacggaatgatcctcctgcatgcatcttgttttgtgatgagttttggggcatgcaacattgggaagtgttgctttgtgtttggtttgtggttttttgccgtttataccattgcagttgtagactcgagcttgtgctttcacagtggggttgcttgtttccattgtagtgttacggttgtgttttaccttgcaagatcatttaagtttagccaacccgtgtggagtgcaggaaggaggacttcccaccttggagggatgaaaggctgcacatttaacactcttcagatgaatagattcatatgtgatgtcgctcagacacaataagggcttgttttacatgacggtgggtactaagaataagtcattttgttccgcactttaaagctttcttaaaaactgtgttgacacttgtgtgtctccgtgcgaacagtcatggcctcctgacttacctcaggcttccaggaagaacaagatgaccacatactgtccccagaacactgccacctttcctcttccgggataacattctcagcgcacttctaagagacaatttttaaagttatttattgaagaaaaaaagttctatgcttttaacattgtaacaaattgacctatgcaggccctaattggttttattttctatccattcaacatttaccccaagtacccagtttttataatttatataaagtcagattgcaatgtttcttactttccgtcgttttgtagatcatttttttaatactgtgtaaaacttttttttttttttttttacacctgagctgtggttttgatactgatattttcctatgccggaaacttttcttgctttcagggaaggtaagaaaatgtgttttttacatttgttacttatgtaacattcatattttcacagtttgatatctgtaacatactgtatgctttctacctgtaaatgccaacaatagaattaaaatatttatttaaaa

SEQ ID NO: 2 - DNA, 16120 bp, homo sapiens
  misc_feature: human Fat4 cDNA
atggacttagcaccagacagggctactggccgcccgtggctcccgttgcacactctatcagtatctcagctccttcgagtgttttggctactgtcattgcttccggggcaggcctgggtccacggggccgagccgcgccaggtgttccaagtgctggaagagcaacctccaggcactctggtaggcaccatccagacgcgccccggcttcacctacaggctcagcgaaagccacgccctgtttgccataaacagtagcaccggagccctgtacaccacctccaccatcgaccgcgagagcctgcccagcgacgtgatcaacctggtggtcctttccagcgcgcccacctaccccaccgaagtgcgagtgctggtgcgggacctcaatgacaacgcccccgttttcccggacccctctatcgtggtcactttcaaggaagacagtagcagcggacgccaagtcatcttagacaccgccaccgactcggacatcggctcaaacggtgtggaccaccgctcctaccgcatcatccgcggcaatgaggcggggcgcttccgtctggacatcaccctgaacccgagcggcgagggagcgttcctgcatctggtgtccaagggcggactggaccgtgaggtcactccgcagtaccagctcctggttgaggtggaggacaagggtgagcctaagcggcggggctaccttcaggtaaacgtgactgtgcaagacattaatgacaaccccccggtttttggcagttctcactaccaggcgggggtgcctgaggacgcggttgtgggttccagcgtcctccaggtggcggcggcggacgcggacgagggcaccaacgcggacatccgctatcgcctgcaggacgaggggacccccttccaaatggaccctgagacgggacttatcacggtgcgggagcccctggacttcgaagctcggcgccaatactcgcttacggtgcaggcgatggacagaggcgtgccttccctcactgggcgcgccgaggcgctgattcagctgctggacgtgaatgacaatgacccggtagtgaagttccgctacttcccggccacctcgcgctacgcctcggtagatgagaatgctcaagtgggcaccgtggtggctctgctcaccgtgacggacgcagattctcccgcggccaacgggaacatctccgtgcaaattctcgggggcaatgagcagcgccactttgaagtgcaaagcagcaaagtgccgaacctgagcctaatcaaggtggccagcgccttggaccgcgagcgcatcccttcctacaacctcacagtttccgtctctgataactacggggcgccccctggcgcagcagtccaggcgcgctcttctgtggcaagcctggtgatttttgttaatgacatcaatgaccatcctcctgtcttttcacagcaagtgtacagagtgaacctgagcgaggaggcgcctccgggaagctatgtgagtgggatatctgccactgatggcgactctggtctcaatgctaatctgcgttacagcattgtctctggcaatggactgggatggttccatatcagtgaacatagcggcctcgtgaccactgggtcctctgggggcctggaccgtgaacttgcttcccagattgttctgaatataagtgcccgggaccagggagttcaccccaaggtgtcctatgcccagcttgtagtaactctcctagatgtgaatgatgaaaagccagtatttagccagccagaagggtatgatgtgtctgtggttgagaatgccccaacagggacagaactgttgatgctcagggcaactgacggggacctgggtgacaacggaacagtgcgcttctccttacaagaggcagagactgaccggaggtccttccgtctggatcctgtgtctgggaggttgagtactatttcctccttggacagagaagagcaagccttctactccctgttggttctggccacagatctgggctcccctccccagtcatcaatggctcgcataaatgtgagtcttctggatataaatgataacagccctgtcttctacccggtccaatactttgctcacattaaggagaatgagcctggaggtagctacatcaccactgtgtctgccactgacccagacttgggtaccaatggtactgtcaaatatagcatatctgctggggacaggtctcggtttcaggtcaatgctcagagtggggttatttctacaagaatggccctagacagagaagaaaaaacagcttatcagttgcaaatagtagctactgatggtggcaatttacaatctcccaaccaggcaatagtaaccatcactgtattggacactcaagacaacccacctgtattcagtcaggttgcctacagctttgtggtttttgagaacgtggcgctgggatatcatgtgggtagtgtgtctgcatccaccatggatctcaattccaacatcagttatctcattactactggggatcagaaaggtatgtttgctatcaaccaggtcactgggcagcttaccacagcaaatgtgattgatagagaagagcaatccttttatcagctgaaggtagtggccagtgggggcacagtgactggagacactatggttaacataacagttaaggatttgaatgacaactctccccatttccttcaggcaatagagagtgtaaatgtggtggagaattggcaggcaggtcacagcattttccaggccaaagctgtggaccctgatgaaggtgtcaatggcatggtactctatagtctgaagcaaaaccccaagaacctgtttgctatcaatgaaaagaatggcactattagtctgcttgggcccctggatgttcatgctggctcctaccaaatagagatcttggcatctgacatgggtgtcccacagctctcctctagtgtcatcttaacagtttatgtccatgatgtaaatgacaattcaccagtgtttgaccaactctcttatgaagtcaccctttctgagtcagaacctgtgaattctcgattctttaaagtacaagcttctgataaggattcaggagcaaatggtgaaattgcatacaccattgctgaaggaaatacaggggatgcttttggcatattcccagatggtcaattgtatataaaaagtgaactggaccgtgaacttcaagacagatatgttttaatggttgttgcttctgacagagcagtggaaccccttagtgctactgtgaatgttactgtaattttagaagatgtaaatgataacagacctctttttaacagtaccaattacacattttacttcgaagaagagcagagggctgggtcgtttgtgggcaaagtaagtgctgtagataaagactttgggccaaatggagaagtaaggtattcttttgaaatggtgcagccagattttgagttgcatgccatcagtggggaaattacaaatactcatcagtttgacagggagtctcttatgaggcggagagggactgctgtgtttagctttacagtcatagcaacagatcaggggatccctcagcctctcaaggatcaggccactgtacatgtttacatgaaggatataaatgataatgctcccaaatttttaaaagacttttaccaagctacaatatcagaatcagcagccaatctgacacaagtgttaagagtatctgcctcagatgttgatgaaggtaataatggacttattcactattctataataaaaggaaatgaagaaagacagtttgctatagacagtacctctggtcaggtaacactaattggcaaattagactatgaagcaacacctgcctattcccttgtaattcaagcagtggattcagggacaatccccctcaattcaacgtgtactttaaatattgatattttagatgaaaatgacaataccccttctttccctaaatcaacactctttgttgatgttttggaaaacatgagaattggtgaactcgtgtcctctgttactgcaactgattccgattcaggtgacaatgctgatttatattacagtattactgggactaacaaccacggaacttttagcattagcccaaacactgggagtatttttcttgccaaaaaactggactttgaaacacagtctttgtataaattaaatataacagcaaaagaccaaggaagacctcctcgttcatctacaatgtcagtggttattcacgtgagggactttaatgacaatcctcctagctttcctcctggagatattttcaagtctattgttgagaacattcccatcggtacatctgtcatttcagtgactgcacatgaccctgatgcagacattaatggtcaactatcctacacaatcattcaacagatgccaagaggcaaccactttaccatagatgaagtcaaagggactatatatactaatgctgaaatagatcgggaatttgctaatctctttgagttgactgtaaaagccaatgatcaagctgtgccaatagaaactagacggtatgctttgaagaacgtgaccattttggttacagacctcaatgacaatgtcccaatgtttatatcacaaaacgcccttgctgcagacccatcagctgtgattggttccgttctgacaacaattatggctgctgacccagatgaaggtgctaatggagaaatagagtatgagatcatcaatggggacacagacaccttcattgttgatcgttatagtggagacctgagagtggcttcagcgttggtgccttcacagttgatctacaatctcatagtttcagcaacagaccttgggcctgaaaggaggaaatcgaccactgaattgaccatcattcttcagggccttgatggacctgtttttactcaacccaaatatataactattttgaaggaaggagaacccattggcacaaacgtgatatcaatagaagcagctagccccagaggatctgaggccccagtggagtattatattgtttcagttcgttgtgaagaaaaaactgttggacgcctctttactattggacgacatactggtataattcagaccgcagccattctggaccgggagcaaggagcatgtctttacctggtggatgtttatgccatagaaaaatcaactgcttttcccagaacacagagagcagaggtagaaataacacttcaggatatcaatgacaatccaccagtatttccaacggacatgctggatctcacggtagaggagaacattggagatggctctaagattatgcagctgacagccatggatgctgatgagggtgcaaatgctctcgtcacatacactatcattagtggagctgatgatagttttcgcatcgacccagaatccggagatctgatagcaaccaggcggttggacagggaacgccgctccaaatattcactgctagttcgtgctgatgatggtcttcagtcctcggatatgagaattaatatcactgtcagtgatgtgaatgaccatacacccaaattttccagacccgtgtactcttttgacattcctgaggacacaatccctggttctttggtagcagccattttagccacggatgatgactctggtgtgaatggagaaattacatatattgtgaatgaagatgatgaagatggcatctttttcctgaatcctattactggggtctttaatttgactcgattattagattatgaagtacagcaatattatatcctcactgttcgagcagaagatggtgggggacaatttactaccatcagagtttatttcaatattctagatgtaaatgataatccacctattttcagcttgaattcatacagcacatctttaatggagaatctacctgtgggatctactgttcttgtgtttaatgttactgatgcagatgatggcatcaactctcaattgacttatagcattgcttcaggtgatagccttgggcagtttactgttgacaagaatggtgtactcaaagtcctaaaagctttggatcgggaaagtcagtccttctacaacttggttgttcaagtgcatgacctgccacagattccagcctccagattcacaagcactgctcaagtctccattattttgttggatgtaaatgataacccaccgacatttctttcccctaaattgacatacattccagaaaatacacctattgatactgttgttttcaaagctcaagcaactgacccagatagtggcccaaacagctatattgagtacactctgctgaaccctttgggaaacaagttcagtattgggaccattgatggtgaagtgaggctcactggagaactggacagagaagaagtttctaattatactctaacagtggtggctacagacaaaggtcaaccatctctctcttcatctacagaggttgtagttatggtacttgacatcaatgataacaaccccatctttgcacaagctttgtataaagtggagattaatgaaaacacacttactggaacagatataatacaagtgttcgcagcagatggagatgaaggcacaaatggacaggttcgctatggcattgttaatggtaataccaatcaggaatttcggatagactctgtcacaggtgccatcactgtcgctaaacctttggatagagaaaagacccctacctaccatttaactgttcaggcaacagatcgaggcagcacacccagaactgatacctccacggtcagcattgttctactggatattaatgactttgttcctgtatttgagctatctccatattctgtaaatgtccctgagaatttagggacactacccagaacaattcttcaggtggtggcaagagatgatgatcgaggatctaacagcaaactctcatatgttctgtttggtggtaatgaagacaatgcttttactctctcagccagtggagaacttggagtaacacagagtctggatcgggaaacaaaagagcgctttgtcttaatgattacagctacagattcaggatcccctgccttgactggaactggaacaatcaacgtcatagtagatgatgtcaatgacaatgtccccacatttgccagtaaagcgtatttcacaacaattcctgaggatgcaccaactggaacagatgttttattggtaaatgcctcagatgctgatgcttcaaagaatgcagttataagttataggatcatcggtggaaactctcagttcacgatcaacccatcgacaggacaaatcatcaccagcgcattgttagatagggaaacaaaagataattatactttggtagtggtctgcagtgatgcgggatccccagagcctctttccagttccaccagtgtgcttgtcactgtgactgatgtcaatgacaatccaccaagatttcagcatcacccatatgtcactcacatcccatctcctactcttccaggttcctttgtctttgcggttacagtcacagatgctgatattggaccaaattctgaactgcattattctctttcgggtagaaattctgaaaaatttcacattgacccactgaggggagccattatggccgccggaccactaaacggagcttcagaagtgacattttctgtgcatgtaaaagatggtggctcatttccaaagacagattctacaacagtgactgttagattcgtgaataaggccgatttccctaaagtgagagccaaagaacaaacgttcatgtttcctgaaaaccaaccagtcagctctcttgtcaccaccatcacaggatcctctttaagaggagaacctatgtcatattatatcgcaagtgggaatcttggcaatactttccagattgatcagttaacagggcaggtgtctattagtcaacctctggattttgaaaagatacaaaaatatgttgtatggatagaggccagagacggtggtttccctcctttctcctcttacgagaaacttgatataacagtattagatgtcaatgataatgccccaatttttaaggaagacccatttatatctgaaatattggaaaacctttcccctcgaaaaatacttactgtttcggcaatggacaaggacagtggacccaatggacagttagattatgaaattgttaatggcaacatggaaaatagtttcagtatcaatcatgctactggtgaaattagaagcgttagacctttggacagggaaaaagtatctcattatgtcctaaccataaaatcatcagacaaagggtccccgtctcagagtacttcagtaaaagtcatgattaacattttagatgaaaatgataatgcccctaggttttctcagatatttagtgcccatgttcctgaaaattcccccttaggatacacagttacccgtgtcacaacttctgatgaagacattgggatcaatgcaattagtagatattctataatggatgcaagtcttccatttacaattaatcccagcacaggggatattgtcataagcagacctttaaatagggaagatacagaccgttacagaattcgagtttccgcacatgattctgggtggactgtaagtacagatgtcacaatatttgtgacagacatcaatgacaatgctccaagatttagcagaacttcctattatttagattgccctgaacttactgagattggctccaaagtaactcaggtatttgcaacagatcctgatgagggatcaaatggacaagtgttttatttcataaaatcccaatcagaatatttcaggattaatgccaccactggagagattttcaataaacagatcttaaaataccaaaatgtcactggcttcagtaatgtgaatatcaacaggcatagttttatagtgacatcttcagatcgaggtaaaccttccttaattagtgagacaacagttaccatcaatatagtggacagtaatgacaatgcacctcaatttcttaaaagtaaatatttcactccagtcaccaaaaatgttaaggttggtacgaagttaatcagagttacagcaatagatgacaaagattttggactgaattcagaagtggagtatttcatttctaatgataaccatttaggaaaatttaagttggacaatgatacggggtggatttcagtagcatcctccctgatttctgacttgaaccaaaacttttttatcacagtcactgcaaaggataagggaaaccctccactttcttcccaagcaactgttcacataactgtcactgaggaaaactaccatacacctgaattctctcaaagccacatgagtgcaaccatccctgagagccatagcattgggtccattgtcagaactgtttctgcaagagatagagatgcagcgatgaatggcttgattaagtacagcatttcttcaggaaatgaagaaggcatttttgcaatcaattcttctacaggtatattaacactagccaaagctcttgattatgagctatgccagaaacacgaaatgacgattagtgctatagatggaggatgggttgcaagaactggttactgcagtgtgaccgtaaatgtgattgatgtgaatgataattctccagtattcctctctgatgactatttccctactgttttggaaaatgccccaagtggaacaacagttatccacctaaatgcaacagatgctgactctggaacaaatgctgtgattgcgtatactgtacagtcatctgacagtgacctctttgtcattgaccctaacacaggagtcataaccactcaaggcttcttggattttgaaaccaagcagagctaccatcttactgtgaaagccttcaatgtccccgatgaggaaaggtgtagctttgccactgttaatatacaattaaaagggacaaatgaatatgtgccccgttttgtttccaaactttactattttgaaatctcagaagcagctcctaaaggtactattgttggagaagtgtttgctagcgaccgtgatttgggcactgatggggaggtacactatttgatttttggtaatagtcgaaagaagggtttccagatcaataagaagactggacagatttatgtttctggaattcttgatcgagaaaaagaagaaagggtgtctttgaaggtattggccaagaactttggcagcattagaggtgcagatatagatgaggtcactgtaaatgtcaccgtgcttgatgcaaatgacccacccatttttactctaaacatctacagtgtgcagatcagtgaaggggtcccaataggaactcatgtgacctttgtcagtgcctttgactcagactccatccccagctggagcaggttttcttacttcatcggatcagggaatgaaaatggtgccttttctattaatccgcagacaggacagatcaccgttactgcagaattagatcgagaaacccttcccatctataatctctcagttttggctgttgattcagggaccccctcagctacaggtagtgcctctttattagtcaccctggaagatataaatgataacgggcccatgctgactgtcagtgaaggagaagtcatggaaaacaaacggccaggcactttggtgatgacccttcagtccactgaccctgatctccctccaaatcaaggtccctttacttattacttgctgagcacaggtcctgccaccagttatttcagtctgagcactgctggagttctgagcacaaccagagagattgacagagagcagattgcagacttctatctgtctgtggttaccaaggattctggtgttcctcaaatgtcttccacaggaactgtgcatatcacagttatagaccaaaatgacaatccttcacagtctcggacggtggagatatttgttaattattatggtaacttgtttcccggtgggattttaggctctgtgaagccacaggatccagatgtgttagacagcttccactgctcccttacttcaggagttaccagcctcttcagtattccagggggtacttgtgatctgaattcccagccaaggtccacagatggcacgtttgatctgactgtccttagcaatgatggagttcacagcacagtcacgagcaacatccgagttttctttgctggattttccaatgccacagtggataacagcatcttacttcgtctcggcgtaccaacagtaaaggacttcttgaccaaccactatcttcattttttacgcattgccagctcacagctgacaggcttagggactgctgtgcaactgtacagtgcatatgaagagaacaatagaacgtttcttttggcagctgtgaagcgaaatcataatcagtatgtgaatcccagtggcgtagccaccttctttgaaagcatcaaagagatccttctccggcagagtggagtaaaggtggaatctgtggatcatgactcctgtgtgcatggcccatgtcagaatggagggagctgtctacgaagattggctgtgagctccgtattaaaaagccgtgagagtcttccagtcatcatcgtggcaaatgaacctctgcagcctttcttatgcaagtgtctgccaggatatgcgggtagctggtgtgaaatagatatagatgaatgtcttccatcaccttgccacagtggtggaacctgtcacaatttagtgggaggattttcatgcagctgcccagatggcttcactggtagggcgtgtgagagagatatcaatgagtgcctgcagagtccttgcaagaatggtgccatctgccagaattttccaggaagcttcaactgtgtttgcaaaactggatacacagggaaaatgtgtgaatcttcagtcaattactgtgaatgcaacccctgctttaatggtggttcctgccaaagtggtgtggattcttattattgtcattgtccatttggtgtctttggaaaacactgcgagttgaacagttatggatttgaggagttatcatacatggaatttccaagcttggaccccaataacaactatatttatgtcaaatttgccacgattaaaagtcatgccttattgctttacaactatgacaaccagacaggcgaccgggctgagtttttggcccttgaaattgccgaagaaagactaagattctcttataatttaggcagtggtacatataagctcaccaccatgaagaaggtgtcagatggacattttcacactgtgattgccaggagagcaggaatggcagcctccttaactgtggactcctgttctgagaaccaagagccaggatattgtactgtcagtaatgtggcagtttcagatgactggactcttgatgttcagccaaatagagttacagttggaggtatcagatctctagaaccaatccttcagagaagaggacacgtggaaagccatgattttgttgggtgtataatggagtttgcagtcaatggaaggcctctggaacccagccaagctttggcagcacaaggcatcctagatcaatgccctaggctggaaggcgcttgtactcgcagcccatgccaacatggtggcacatgtatggattactggtcatggcagcagtgtcattgcaaagagggactcactgggaaatactgtgaaaaatctgttactcctgacactgccttatcattagaaggcaaagggcgcttggactaccacatgagtcagaatgagaagcgggaatatttgttaaggcaaagcttacgaggtgccatgttggagccttttggtgtgaacagtctggaagtaaaatttaggaccagaagcgagaatggcgttttaatccatatccaagaaagcagcaattacactactgtgaagattaagaatggcaaagtatattttacatccgatgcaggaattgctgggaaagtggagagaaatattcctgaagtatatgttgcagacggccactggcacacttttctaattgggaaaaatggaacagcaacagtattgtctgttgacagaatatataacagagatattatccaccctactcaggacttcggtggccttgatgtgcttactatatcacttggaggaattccacccaatcaagcacatcgagatgcccaaacagcaggttttgatggctgcattgcttctatgtggtatggtggagaaagtcttcctttcagcgggaagcatagcttggcctccatctcaaaaacagatccctcagtgaagattggctgccgtggcccgaacatttgtgccagcaacccctgctggggtgatttgctgtgcattaatcagtggtatgcctacaggtgtgtccctcctggggactgtgcctcccacccgtgccagaatggtggcagctgtgagccaggcctgcactccggcttcacctgtagctgcccagactcgcacacgggaaggacctgtgagatggtggtggcctgtcttggcgtcctctgtcctcaggggaaggtgtgcaaagctggaagtcctgcggggcatgtctgtgttctgagtcagggccctgaagagatctctctgcctttgtgggctgtgcctgccatcgtgggcagctgcgcaaccgtcttggccctcctggtccttagcctgatcctgtgtaaccagtgcagggggaagaaggccaaaaatcccaaagaggagaagaaaccgaaggagaagaagaaaaagggaagtgagaacgttgcttttgatgaccctgacaatatccctccctatggggatgacatgactgtgaggaagcagcctgaagggaacccaaaaccagatatcattgaaagggaaaacccctaccttatctatgatgaaactgatattcctcacaactcagaaaccatccccagcgcccctttggcatctccagagcaggagatagagcactatgacattgacaacgccagcagcatcgccccttcggatgcagacatcattcaacactacaagcagttccgcagccacacaccaaaattttcaatccagaggcacagtcccctaggctttgcaaggcaatcccccatgcccttaggagcaagcagtttgacttaccagccttcatatggtcaaggtttgagaaccagctccctaagccactcagcatgcccaactcccaaccctctgtctcgacacagtccagcccctttctccaaatcttctacgttctatagaaacagcccagcaagggaattgcatcttcctataagggatggtaatactttggaaatgcatggtgacacctgccaacctggcattttcaactatgccacaaggctgggaaggagaagcaagagtcctcaggccatggcatcacatggttctagaccagggagtcgcctaaagcagccgattgggcagattccactggaatcttctcctccagtcggactttctattgaagaagtggagaggctcaacacacctcgccctagaaacccaagtatctgcagtgcagaccatgggaggtcttcttcagaggaggactgcagaaggccactgtctagaacaaggaatccagcggatggcattccagctccagaatcctcttctgatagtgactcccatgaatctttcacttgctcagaaatggaatatgacagggagaagccaatggtatatacttccagaatgcccaaattatctcaagtcaatgaatctgatgcagatgatgaagataattatggagccagactgaagcctcgaaggtaccacggtcgcagggccgagggaggacctgtgggcacccaggcagcagcaccaggcactgctgacaacacactgcccatgaagctagggcagcaagcagggactttcaactgggacaaccttttgaactggggccctggctttggccattatgtagatgtttttaaagatttggcatctcttccagaaaaagcagcagcaaatgaagaaggcaaagctgggacaactaaaccagtccccaaagatggggaagcagaacagtatgtgtgaagtttatgtactggcactataaaatataaaaacaagaaataatactcaaaccattgtaaagttgctgactaggttgggtcacatttgaaaaacaggccagtatggactagtggtggagggaaaactttaaaaataataaccacaatgctgctgaaacagactcacaacaactcttaatttaaacatgtgtggttgaatttatttccctgcatgcattgtgttttgtaactagttatgtggcatgcagcatttggaaaatttttcttatttaccagtgtttgatttgtgatttttaaaaattgatacctttaccattgcagaaaagaacttgtgctttcccagtggcgtatgtgtattgtttcaactgtattattataattatattttgcattgcaagattcttgatgttaaaccaatccttgtaaagtgtaaaaaggaaccctcctatcgtggaatgaaagattaagtatattaacacttttcagaatgatagtttctgtatttgatgttgctcagaaatgtctgagtatttgagtaagttttacatgacagtgggtactgaaattaagtcattttgttcagcactttaacgctttcttatagaattgtcttaaaacttctggatccttgagcaaatgattatagtctcctgactttcatgaggcttccattaggaacagaatgattgcatgttgtccccagaacactgccaccttgctatgcgaatgatgttctcagcagcacttctaagaaacactcttaaaagttatttattgaaaatttttcgtatgcttttaatattttaaagaattgacctaaggaaagcttatgattggacttattttccaaccagataacatttactctaagtacccagtttttataatttatatgaaatcagatttcaacacttactttgtcattttgtagatcattttttttaaatactgtgtaaaaactttttttacacctaagctgtgtttttgatactgatattttcctatgctgaatagttttcttactttcagggaaggtaagaaaatactttttttatatttgttacttatgtaacattcatatttttctcattttgatatttgtaacatactgtatgctttctacttgtaaatgtcaacaatagaattaaaatatttatttaaaataaaaa

SEQ ID NO: 3 - DNA, 16066 bp, rattus norvegicus
  misc_feature: Rat Fat4 cDNA
atgaacttggccgcaaaccggactccgggccgcaggcggttcccgctaccttccccatctctgggccagcttctgagagtctggggactgctgtcgctgctccccagtccggcccgagtccaagcggccgaacagcgccaggtgtttcaggtgatggaggagcaacccccagggactctggtgggcaccatcccaacgcgtcctggcttcacctatcggctcagtgaaagccacgccctgttcgctattaacagcagcactggagcgttgtacaccaccgctaccatcgaccgcgagagcctgcccagcgatgtggtcaacctggtggtcctttccagctcgcccacctaccccaccgaggtgcgcgtgctggtgcgtgacctcaacgacaatgccccagtcttcccagacccctcgatcgtggtcaccttcaaggaggatagtggcagcgggcgtcaggtcattctggacactgccaccgactctgacatcggctccaacggcgtggaccaccgctcctaccgcatcgtccgcggcaacgaggcgggccgctttcgcctggacatcactttgaaccccagtggcgagggagcattcctgcacctggtgtccaagggtgggctggaccgcgaggtcacacctcagtaccagctcctggtagaggtggaggacaagggtgagccaaagcggcggggttaccttcaggtcaacgtgactgtacaggacattaacgacaatcccccggtgtttggcagttcccattaccaggcgggggtgcctgaagacgcagttgtgggttccagtgtcctgcaggtggcggcggcggacgcagacgagggcaccaatgcggacatccgttaccggctgcaggacgagggaacccccttccagatggacccggagacggggctcatcaccgtgagagagccgctagactttgaggcccggcgacagtattcgctgacagttcaggccacggaccgaggcgtgccctcgctcaccgggcgtgccgaagcgcttatccagctgctagatgtcaatgacaacgaccctgtagtgaagtttcgctacttccctgccacctcgcgctacgcttcggtggatgagaacgcgcaggtgggtaccgtggtggctctgctcaccgtgaccgatgctgactccccagcagccaacggaaacatttcagtgcagattctcgggggtaatgagcagcgccactttgaggtacagaggagcaaggtgcccaacctgagcctcatcaaggtggccagcgccctggaccgcgagcgcatcccttcatacaatctcacagtctcggtctcggataactccggggcgccccctactgccgaggtccaggcccgctcctctgtggccagcttggtgatttttgtcaatgacatcaatgatcaccctccggtctttgagcagcaagtgtacagagtgaacctgagtgaggaagtacctccggggagctatgtgagtggggtgtctgccaccgatggtgactctggtctgaatgctaacctgcgttatagcattgtctctggaaatgggttgggctggtttcacattagtgagcacagtggcctggtgactaccagcgctgctgggggccttgaccgtgaacttgcttcccagattgtactgaatatcagtgcccgagatcagggagttcaccctaaggtatcctatgcccagcttgtggtcagtgtcctggatgttaatgaccaaaagcccgtgtttagccagcctgaaggctatgaggtgtctgtggttgagaatgctccaactgggacagagctgttggttcttggggcaacagatggggatctgggtgacaatgggacagtgcgcttctccctgcaagaggctgagagtgaccaaaggctcttccgcctggatccagtatctgggaggctgagtactatctcttccctggacagagaggaacaagccttctactgtctattgattctagccacagacctgggttcccctccccagtcctccaccgtgcaggtaaacgtcagtctccttgatataaacgacaacagccctgtgttctacccagtccaatactttgctcatatccaggaaaatgagcccggaggtagctatgtcaccacagtgtctgctactgacccagacatgggtcccaacggaactgttaagtacagtatatcagctggggacaggtctcggtttcagattcatgctaagagtggagtcatttctacaaagatggccctagacagagaagagaaaacagcataccagctgcaaatagtggccaccgatgggggtaatttacagtcccccagccaggccatagtaacagtcacagtgctagatacacaggacaacccacccgtattcagccaggctgcctatagctttgtggtctttgagaatgtggctctggggtatcatgtgggcagtgtttctgcaaccaccatggaccttaatgccaacatcagttacctcatcaccactggggaccagagaggtatgtttgctatgaatcctgttacagggcagctgaccacagcaagtgtcattgacagggaagaacagtccttttatcagctgagagtggtggccagtgggggggcagtcacaggagatgctgtggttaacatcacagtgaaagatttgaatgacaacgccccccacttcctccaggcagtggaaagcatagatgcagtggagaactggcaggcaggacacagcatcttccaggccaaagccgtggaccctgatgaaggtgtcaacggtagggtgctctatagtctgaagcagaaccccaagaacctgtttactattaacgagcagaatggtaacatcagtctgctgggtaccctagatgtccatgctggctcgtaccaggtagaaatcgtggcctccgatatgggggtcccccagctctcctccagcatcattctgacagtttatgtccacgatgtaaatgacaacccgccagtatttgatcagatttcctacgaggtcaccctttctgagtcagagcctgtgaactctcgattctttaaagtacaagctttggataaagattccggagcgaatggggagatagcatacgcgatcacagatgggaacagtggggaggcctttggcatattcccagatggtcagctgtatataaagagcgaactagatcgcgaacttcaggacagatatgtgttgctggttgttgcctctgaccgggcggtagagcctctaagtgctactgtgaacgtcactgtgattttagaagacgtgaatgacaacaggcctttgtttaatagcaccaattacacattttacttcgaagaggagcagagagccgggtcattcgtaggcaaagtcagcgctgtggacaaagactttgggccgaatggggaagtaaggtatgcttttgaggtggcacagccagatttcgagctgcatgccatcactggggagatcaccaacactcacaagtttgacagagagtccctcatgaggagaagggggacagcagtgttcagcttcacagtgatagccacggatcagggcctccctcagcctctcaaggaccaggccactgtgcatgtctacatgaaggacatcaacgataatgcccccaagtttctaaaagacttttatcaagctacagtatcagagacggcaaccaatctgacccaggtcttacgagtgtctgcctcggacgtggatgaggggaataatggcctcattcattactctatcctcaaaggaaatgaagagagacagttttccatagacaggttctctggccaggtgactctcgtaggcaagttagactatgaagcaaccccagcctattccctcctcatccaggcggtggattcagggaccgtccccctcaactcgacctgtaccctgagtattgatattttggatgagaatgataacactccctctttcccgaagtcaacactctttgtggatgttttggaaaacatgaggattggggaacttgtatcctctgtcactgccactgactcggactcaggtgtcaatgccgatctccactacactattacagggtcaaacaaccacgggacttttagtattagtccaaacaccgggagtatctttctggccaaaaagttggactttgagacacagtctttatataaattaaacataacagcgaaagaccaggggcggcctcctcggtcatccacaatgtcagtggtgatccatgtgagggacttcaatgacaaccccccgagctttcctccaggagacatttttaagtcaatcgtggagaacatcccccttggtacatctgttatttctgtgacagcacatgacccagatgcggacatcaatgggcagctgtcttatgccatcatccaacagatgccccgaggcaaccacttcagcatagacgaagtcaaaggaaccatctacaccagtgctgagattgacagggaattcgccaacctctttgagttaacggtaaaagccaatgaccaagctgtgccgatagaaaccaggaggtatgctttgaaaaatgtgaccattttggtgacggacctcaatgacaatgtcccaatatttatatcacaaaatgcactggctgcagacccttcagccatgattggttctgtcctgactaccatcatggctgcagacccggatgaaggtgccaatggagaggtggagtatgagatagtcaatggggacacagacaccttcactgtggatcgttacagtggagaccttcgagtggcttctgcactggttccttctcagctcatttacaaccttatagtatcagccacagaccttggcccagagagaaggaagtcaaccacagaactgactgtcatccttcagggcctagatgggcctgttttcacacagaccaaatacataaccatcttgaaagaaggggagccaattggcaccaatgtcatctccatagaagcagccagccccagagggtcagaagctccagtggaatattacatcgtttctgttcgttgtgaagagaagactgtcggacgtctgtttaccattggacgtcagactggtgtcattcaaactgcagccattttggaccgggagcagggagcatgcctctacctggtggatgtttatgccatagagaagtcgtctgcttttcccaggacacagagagcagaggtagaaataacacttcaggatatcaatgacaacccaccagtgtttccaacagacaccctggatctcactgtggaagagaacattggagatggctctaagattatgcagctgactgccatggatgctgacgagggtgcaaatgctttggtcacgtatgctctcattagtggagctgatgacagcttccggattgaccctgaatctggagacctgatagccaccaagaggctggatagggagcacaggtccaagtactcattgctggttcgtgcagatgacggcctccagtcctccgacatgaggataaacatcaccatcagcgatgtgaatgaccacacccccagattctccagacctgtgtactctttcgacatcccagaagacacaacccctggttccctggtggcagctattttagcaacagatgacgactctggtgtgaatggagagatcagctatgtggtggaggaagacgatggggatggtgtatttttcctgaatccagttactggtgtcttcaacttgacccgagcactggattatgaaaggcagcagtattacatactcacagtccgcgctgaggatggcggaggacagtttaccaccatccgagcttacttcaacatacttgacatcaatgacaatccgcctgtcttcagcctgagctcctacagcacatcgttaatggaaaacctgcctctgggatccaccgtccttgtgtttaacgtcaccgatgcagatgacggtgtcaactctcagctgtcctacagcattgcttcaggggacagccttggacagtttgcagtggataagcatggagtcctcaaagccctcaaagctttggaccgggaaagtcagtccttctacaacctggtcattcaggtccatgacttgccccagcctcctgcctccagattcacaagcacagcccaagtctccatcatcttgttagatgtcaatgataacccacccatgtttctttcccccaagttgacgtacattccagaaaacacacccattgatactgttgtcttcaaggctcaagcgacagacccagacagtggccccaacagctatattgagtatactctcctgaaccctgcaggaaacaagttcagcattgggaccattgatggtgaggtgcatcttactggagagctggacagagaggaagtttccaattacagcctaacagtggtggccacagacaaagggcaaccacctctgtcgtcctctactgaggttgtggttatggtccttgacatcaatgacaacaacccagtttttgcccaggctatgtaccgagtgcagattaaggagaacacactcactggaacagatataatccaagtgtctgcagcagataatgatgaaggcaccaatgggcaggttcgctatggcatcgtgggtggaaacacacatcaggagttcaggatcgactcggtgacaggggccatcacagtggctaagtctttggatagagagacaatccctgcttacattttaactgttcaggccacggatcggggcagtagccccagaacagattcctgcacggtcgccatcactttacttgacatgaatgattttgttcctgtatttgagctgtctccgtattctgtgaatgttcctgaaaatttaggggccctgcccagaaccattctccaggtggtggcaagagatgatgatcaaggaccaaatagccagctctcatatgttttgcttggtggaaatgaagacaatgcctttgcccttactgccagtggtgagcttcgggtgacccagagtctggatcgggaagcaagagatcacttcgtcttggttgtcacagctgcagatgcaggatcaccagccttgacaggaacagggacaatcaacatcattgtagatgatatcaatgacaatgtccccactttcgccagtaacatgtactttactgcaattcctgaggatgccccgacggggacagatgtcctgttggtaaatgcctcagatgcagatgctgcaacaaatgctgtcatcagtatcatcggtggaaactcacagttcaccatcaacccatccacgggacagatcatcactagtgcactgctggacagagaaaccaaagataattacactttggtcgtggttgccagtgatgctggatctccagagtctctttccagctctactagtgtgctggtgtctgttaccgatgttaatgacaacccaccccggttccagcatcacccatatgttacacacatcccatctcctactcctccaggttcctttgtttttgctgtcacggtcactgacgctgacattggacccaattctgaactccattactcactctcaggtagaaactctgaaaaatttcacattgaccctctgaggggggcaatcatggcagctgggccactaagtggagcttctgaggtgacattctctgtccatgtgaaagatggcggctcatttccaaagacagattctaccacagtgactgttaggtttgccaacaaggcagatttccccaaagtcagagccaaagaacaaacattcatgtttccagaaaaccagccagtgggcactctggtcacaaccatcacggggtcctccttgagaggagaaactttgtcctactacatcgcgagtgggaacctgggcaacgctttccaaattgatccactaacaggacaggtgtccatcagccaacctttggactttgagaagatacaaaaatacattgtttggatcgaagccagagatgggggcttccctcccttctcttcttatgagaagcttgacatcacggtgctagatatcaatgacaatgccccggcttttgaagaagatccttttgtgtctgagatcctggaaaacctatcccctcgaaaaatccttactgtttcagcaacagacaaggacagtggtcccaatggacaactagcttatgagattgtgaatggtaaccaggaaagcagtttcacgattaatcacgctacaggtgaaatcagaagcattcggccactagacagagaaaaaatatcttattatgagctcactgtgaaatcttcagacaaagggtctccttctcagagcacctctgtcaaagtaattatcagtgttttagatgaaaatgataatgcacctaggttttctcagatattcagtgcctatgtttccgaaaactctcccttgggatatacagttacccgtgtcacaacttctgatgaagacattggcataaatgcaattagtagatattccatagcggacacagctcttccatttaccatcaaccccaacacaggggatattgtcatcagtcgacctttaaacagagaagatacagatcgatacaggatccgtgtgtccgcacatgattctggctggaccgtaagtactgatgtcaccatattcgtgactgacatcaatgacaacactccaagatttagcagaccctcttattatttagattgccctgaacttactgaacttggctccagagttactcaagtatctgccactgatcccgatgaggggtcaaacggacaagtgttttatttcataaagtcgaagtcagagtatttcagaataaatgccaccactggagagattttcaacaagcaggtgttaaaataccaaaatgtcagtggcttcagtaatgtgaacatcaacaggcacagtttcattgtgacagcttctgatcgaggcaacccttccttactcagtgagaccacggttactatcaacacagtagacagcaatgacaacccacctcagtttctcaaaaataaatactttaccccagttaccaaaaacgttaaagtcggtactaagttaatcaaagtgacagcggtagatgacaaagactttggattaaactctgaagtggagtactttgtgtctaatgggaaccacctagggaagtttaaattagacaatgatactgggtggatttcaatagcgtcttccttggtttctgacctaaatcaaaacttcttcatcagagtaacagcaaaggacaaagggaatcccccactttcctcccaagtagtcattcacattacagtcactgaagagaactaccacacccctgaattctcccagagccatataagtgccaccatccctgagagccacagcattgggtctgtggtcagaactgtctctgccagagatcgtgacacagccatgaacggtctgatcagctacaacatttcttcagggaatgaagagggcatctttgctatcaattcctcaacaggtgtggtaaccctagccaaagcccttgattatgagttgagctcgaagcatgagatgaccattagcgccacagatggaggatgggttgcgaggactggctattgcagcctaactgtgagtgtgatcgacgtgaatgacaattctccagtgtttgttcctgatgaattcttcccaactgttatggagaatgccccaagcgggaccactgttattcacctaaatgcaacagatgctgactcgggagccaatgctgtgattgcatacactgtgcagtcatcagacagtgatctgtttgtcattgatcccaacacaggggtcatcaccactcaaggcttcttggattttgaaacgaagcagagctaccacctcacagtgaaagccttcaatgtccctgatgaggaaaagtgcagctttgccactgttaacatccagttgatgggaaccaatgagtatgtgcctcgctttgtttccaagctctactactttgaagtgtcagaagcggctcctagggggactgctgttggagaagtgtttgccagtgacagggacatgggtgctgatggagaagtgcactatttgatctttggaaacagcaggaagaagggcttccagatccacaagatgacagggcagatctatgtgtctgggcttcttgatagagagaaagaagaaagggtgtccttaaaagtgttggccaagaactttggcaacatccggggcgcagacattgatgaagtcactgtgaacatcacggtgcttgatgctaatgacccacccgtgtttagtctgaacacctatagagtccagatcagtgaaggggtccctattgggacccacgtgacctttgtgagtgcctttgactcagattccatccctagctggagcaggttctcatacttcattgggtctggaaatgaaaatggtgccttttccattaacccacagacaggacagatcacagttacctcagggctggaccgggaaagcctgcctgtttataatctcactgtcttggctgtagactcagggactccctctgccacaggaagtgcttccttgttagttaccctagaagacatcaatgacaatggcccagtgttgactgtcagtgaaggagaagttctagaaaacaaacgcccaggaactctagtgatgaccctgcagtccacggatcctgaccttccccccaatcaaggccctttcaactattacctgctgagcacaggccctgctactaattatttcagtctcaacaccgctggagttttaagcacaacccgggaaattgaccgagagcagattgcagacttctatctgtctgtggtcaccagagattctggggctcctcagatgtcatcgacaggaactgtgcacatcactgttctagatcaaaatgacaacccctcgcagtcccgcacagtggagatatttgttaattattatggcaacttatttcctggcgggactctgggctctgtgaagccccaggatccggatgtgttagacagtttccactgttccctcacatcaggagtcacaagtctcttcagtatcccagcaggcagctgtgacctgagttcccagcccagatccacagacggcaccttcgacctgactgtggttagcagcgatggggttcacagcacagtcaccaacaatatccgagttttctttgcaggattctctaatgctacgatagataacagcattttgcttcgtgtcggtgttcccacagtcaaggacttcctgactaatcactaccttcactttctgcgcattgctagttcccagcttaccggcctgggcactgcggtgcagctgtatgctgcttatgaggagaacaacagaacgtttcttttagcagctgtgaaacgtaatcataaccaatatgtgaaccccagcggtgtagccaccttcttcgaaagcatcaaagagatccttctccggcagagtggagtcaaggtcgaatctgtggatcatgacccatgcattcatgcgccgtgtcagaacggaggcagctgccttcgaaggcttgctgtgggctctgcgctgaagatccaggagagtctgcctgtcatcattgtggccaatgaacctctgcagcctttccagtgtaagtgtgttccaggctatgctggaagctggtgtgaagttgatattgatgaatgccttccagccccttgccacaatggggggacctgccacaacttagttgggggattttcatgcagctgctcggagggcttcactgggagagcctgtgagagagacattaacgaatgtctgcccagcccttgcaagcatggagccgtctgtcagaatttcccagggggattcaactgtgtgtgcaaaactggatacacaggaaaaatgtgtgaatcttcagtcaattactgtgaatgcaatccctgcttcaatggtggttcctgtcagagcggtgtggagtcatattactgccactgtccattcggtgtctttggcaagcactgtgaactgaacagttatggatttgaggaactgtcatacatggagtttcctagcctggaccccaacaacaactatatttatgtcaagtttgctactatcaaaagtcatgctttattgctttataactacgacaaccagacaggagagcgggcagagttcctggccttggaaattgctgaagaaaggctcagattctcatataatcttggcagtgggacatacaaactgaccaccatgaagaaggtgtcagatggacaattccacacagtgattgctcggcgcgcaggaatggcagcctcgttaactgtggactcctgctcggagaatcaagagccaggctattgcactgtcagtaatgtggctgtttcagatgactggactcttgatgtacagccaaaccgagtcactgttggaggaatcagatcactagagccaatccttcagaggaaggggcatgtggagagccacgattttgtgggctgtataatggagttcgctgtgaatggacggcccctggagcccagccaggctctggcagcacaaggcatccttgatcagtgccctagactagaaggcacctgtgctcgaaacccctgccagcacggtggcacctgtgtggacttctggtcatggcagcagtgtcagtgcatggagggcctgactgggaagtactgtgaaaaatctgtcactccagacactgccttgtcactagaaggcaaaggacgcctggactaccacatgagccagagcgagaagcgggaatacttgctgacgcagagcatacgggaagccacattggagccttttggtgtcaatagcctggaagtaaaattcaggactcggagtgagaatggcattttgatccatatccaggaaagcagcaattataccacagtgaagattaagaacggcaaagtgcactttacatcagatgcaggtgttgctgggaaagtggagcggatcatcccggaagcatacgttgcggatggccattggcataccttccggatctcaaagaatggaagctccactgtgctgtctgtagaccggacgcacagcagagacgttgtccaccccacgcaggactttggtggtatcgaggtgctttccatgtccctgggaggaatccctcccaaccaagcccatcgggacacgcagacgggttttaacggctgcatcgcctcagtgctctatggcggagagagccttcccttcagtgggaaacacagcttagcgtccatctccaaaaccgacccttcagtgaagattggctgtcggggtcccaacatctgtgctagcaacccctgctggggcgaccttctgtgtatcaaccagtggtatgcctacaaatgcgtccctcctggtgactgtgcatcccacccttgccagaatggaggtagctgtgagccaggtctgctctctggctacacgtgcagctgtccagagtcacacactgggaggacctgtgagacagtggtggcttgcctcggtgttctctgtcctcagggcaaggtttgcaaggctggaagtcctgggggacacgtgtgtgtccagagtcagggtccagatgagatctccttacctctgtgggctgtgcctgcaatcgtgggcagctgtgccacagccctagcactgctcgtcctcagcttgattctatgcaaccagtgcagggggaagatgcccaaaaaccccaaagaggagaagaagccaaaggagaaaaagaaaaagggcagtgagaatgtggcttttgatgacccagacaacatccctccctatggggatgacctagctgttaggaagcaacccgaggggaatcccaagccggacatcatagagcgagagaacccttacctaatcttcgatgagactgacatcccccacaactcagagaccatccccagtgccccactggcttcccctgagcaggaaattgaacactacgacatcgacaatgccagcagtatcgcgccctctgatgcagacatcatccagcactacaaacagttccgaagccacacgcctaagttttctatccagaggcacagtcctctgggcttcgccaggcagtcccctatgcctttgggagcaagcagtttgacctaccagccttcttcatatggtcaggcattaagaaccagctccctcagccactcggcctgccccactcccaaccccttgtccagacacagcccggctcctttctccaaaccatctgctttctacaggaacagcccagcccgggaactgcatcttcctctgcgggatggggggacgctggaaatgcatggggacccctgtcagcctggcatgttcaactatgccaccaggctaggtagaagaagtaagagcccccaggccatggcatcacatggctcgagacctgggagtcgcctcaagcagcccatagcacagatccccctagaatcttctcctcctgtgggactgtccattgaggaggtagagaggctcaatacccctcgccccagaaaccccagcatctgcagtgcagaccatgggagatcctcttctgaagaagactgtagaaggcctctgtccagaaccaggaacccagcagatggcatccctgccccagaatcttcttctgacagtgactcccatgattccttcacatgctcagaaatggagtacgacagggagaagccggtggtctacacttccagaatgcccaagttatctcaagttaatgaatccgatgcagatgacgaagataattatggagccagactgaaacccagaaggtaccatggacgcagagctgaggggggaccggtgggtacccctgcagcagcatctggggctgcagacagcaccctgaaactgggacagcaggcaggcaacttcaactgggacaatcttttgaactggggcccgggctttggccactatgtggatgtctttaaagatttggcctctctcccagaaaaagcagctggtaatgaagaaggtaaaagcggggcagctaaaccagccgccaaagacggggaagcagaacagtacgtgtgaggtgtatgtgtgggcaagaaacattcaaaaagatggtgtggcttcccaccagcggggtcacctttgtaaaacaggccagtatagaccggtggccagggagggacacgtttaaaattaaccacaaagctgctcacatagactccaagtgactctttaatttaaatgtgctcggtggaacagaatgagcctcctgcatgcatcttgttttgtgatgagttttggggcatgcaacattggggagttttgctttgtgtttggtttgtggttttttgcagtttataccattgcagttgtagacatgagcttgtcttttcacagtggggttgcttgtttccgttgtgttttaccttgcaagatcatttgagtttaaccaacctgcgtaaagtgcaggaaggagcgcttcccatcttggagggatgaaagactgcacattaaccctcttcagacgtatagattcatacgtgatgttgctcagacacgataagggcttgttttacatgacggtgggtactaggcttaagtcattttgttccgcactttaaagctttcttaaaaactgtgtagacacacctgtgtctctgtgcgaactgtcatggcctcctgacttacctcaggcttccaggaagaacaagatgaccacatactgtccccagaacactgccacctttctcttctggaataacgttctcagcgcacttctaagagacaatttttaaagttatttattgaaaaaaaaagttctatgcttttaacattgtaacaaattgacctatgcaggccctaattggttttattttctagccattcaacatttaccccaagtacccagttttcataatttatataaaatcagattgcagcgtttcttactttctgttgttttgtagatcattttttaatactgtgtaaaacttttttttttttacacctgagctgtggttttgatactgatattttcctatgccggaaacttttcttgctttcagagaaggtaagaaaatgtgttttttacatttgttacttatgtaacattcatattttcacagtttgatatctgtaacatactgtatgctttctacctgtaaatgccaacaatagaattaaaatatttatttaaaataccgtg

SEQ ID NO: 4 - protein, 4981 aa, mus musculus
  MISC_FEATURE: mouse Fat4 protein
MNLAANRAPGRRRLPLPSPSLCQLLRVWGLLSLLPGSARVQAAEQRQVFQVMEEQPPGTLVGTIPTRPGFTYRLSESHALFAINSSTGALYTTATIDRESLPSDVVNLVVLSSSPTYPTEVRVLVRDLNDNAPVFPDPSIVVTFKEDSGSGRQVILDTATDSDIGSNGVDHHSYRIVSGNEAGRFRLDITLNPSGEGAFLHLVSKGGLDREVTPQYQLLVEVEDKGEPKRRGYLQVNVTVQDINDNPPVFGSSHYQAGVPEDAVVGSSVLQVAAADADEGTNADIRYRLQDEGTPFQMDPETGLITVREPLDFEARRQYSLTVQATDRGVPSLTGRAEAFIQLLDVNDNDPVVKFRYFPATSRYASVDENAQVGTVVALLTVTDADSPAANGNISVQILGGNEQRHFEVQRSKVPNLSLIKVASALDRERIPSYNLTVSVSDNSGAPPTAEVQARSSVASLVIFVNDINDHPPVFEQQVYRVNLSEEVPPGSYVSGVSATDGDSGLNANLRYSIVSGNGLGWFHISEHSGLVTTSAAGGLDRELASQIVLNISARDQGVHPKVSYAQLVVTVLDVNDEKPVFSQPEGYEVSVVENAPTGTELLVLGATDRDLGDNGTVRFSLQEAENDQRLFRLDPVSGRLSTASSLDREEQAFYCLSILATDLGSPPQSSTAQVNVSLLDINDNSPVFYPVQYFAHIQENEPGGSYVTTVSATDPDMGPNGTVKYSISAGDRSRFQIHAKSGVISTKMALDREEKTAYQLQVVATDGGNLQSPNQAIVTVTVLDTQDNPPVFSQAAYSFVVFENVALGYHVGSVSATTMDLNANISYLITTGDQRGMFAMNPVTGQLTTASVIDREEQSFYQLKIVASGGAVTGDTVVNITVKDLNDNAPHFLQAVESINAVENWQAGHSIFQAKAVDPDEGVNGRVLYSLKQNPKNLFTINEQNGNISLLGALDVHAGSYQVEIVASDMGVPQLSSSILLTVYVHDVNDNPPVFDQISYEVTLSESEPVNSRFFKVQASDKDSGANGEIAYTITDGNNGDAFGIFPDGQLYIKSELDRELQDRYVLLVVASDRAVEPLSATVNVTVLLEDVNDNRPLFNSTNYTFYFEEEQRAGSFVGKVSAVDKDFGPNGEVRYAFEVTQPNFELHAVTGEITSTHKFDRESLMRRRGTAVFSFTVTAMDRGLPQPLKDQATVHVYMKDINDNAPKFLKDFYQATVSETATNLTQVLRVSASDVDEGSNGLIHYSILKGNEERQFAIDSFSGQVTLVGKLDYEATSAYSLLIQAVDSGAIPLNSTCTLSIDILDENDNTPSFPKSTLFVDVLENMRIGELVSSVTATDSDSGVNADLHYTITGSNNHGTFSISPNTGSIFLAKKLDFETQSLYKLNITAKDQGRPPRSSTMSVVIQVRDFNDNPPSFPPGDIFKSIVENIPLGTSVISVTAHDPDADINGQLSYAIIQQMPRGNHFSIDEVKGTIYTSAEIDREFANLFELTVKANDQAVPIETRRYALKNVTILVTDLNDNVPMFISQNALAADPSAMIGSVLTTIMAADPDEGANGEVEYEILNGDTDTFTVDRYSGDLRVASALVPSQLIYNLIVSATDLGPERRKSTTELTVILQGLDGPVFTQTKYITILKEGEPIGTNVISIEAASPRGSEAPVEYYIVSVRCEEKTVGRLFTIGRQTGVIQTAAILDREQGACLYLVDVYAIEKSSAFPRTQRAEVEITLQDINDNPPVFPTDTLDLTVEENIGDGSKIMQLTAMDADEGANALVTYALISGADDSFRIDPESGDLIATKRLDREHRSKYSLLVRADDGLQSSDMRINITISDVNDHTPRFSRPVYSFDIPEDTTPGSLVAAILATDDDSGVNGEISYVVEEDDGDGVFFLNLVTGVFNLTRALDYETQQYYILTVRAEDGGGQSTTIRAYFNILDVNDNPPVFSMSSYSTSLMENLPLGSTVLVFNVTDADDGVNSQLSYSIASGDSLGQFAVDKHGVLKTLKALDRESQSFYNLVIQVHDLPQPPTSRFTSTAQVSIILLDVNDNPPMFLSPKLTYIPENTPIDTVVFKAQATDPDSGPNSYIEYTLLNPSGNKFSIGTIDGEVHLTGELDREEVSNYSLTVVATDKGQPPLSSSTEVVVMVLDINDNNPVFAQAMYRVQIKENILTGTDIIQVSAADNDEGTNGQVRYGIVGGNTHQEFRIDSVTGAITVAKSLDRETTPAYTLTVQATDRGSSPRTDSCTVAITLLDMNDFVPVFELSPYSVNVPENLGTLPRAILQVVARDDDQGPNSQLSYVLLGGNEDNAFVLTASGELRVTQSLDREARDHFVLVVTAADAGSPALTGTGTINIIVDDINDNVPTFANNMYLTSIAEDARTGTDVLLVNASDADAAANAVISYSIIGGNSQFTINPSTGQIITSALLDRETKDNYTLVVVASDAGSPESLSSSTSVLVTITDVNDNPPRFQHHPYVTHIPSPTPPGSFVFAVTVTDADIGSNSELHYSLSGRNSEKFHIDPLRGAIMAAGPLSGASEVTFSVHVKDGGSFPKTDSTTVTVRFANKADFPKVRAKEQTFMFPENQPVGTLVTTITGSSLRGETLSYYIASGNLGDTFQIDPLTGQVSISQPLDFEKIQKYVVWIEARDGGFPPFSSYEKLDITVLDINDNAPTFEEDPFVSEILENLSPRKILTVSATDKDSGPNGQLDYEIVNGNQESSFTINHATGEIRSIRPLDREKISHYELTVKSSDKGSPSQSTSVKVIISILDENDNAPRFSQIFSAYVSENSPLGYTVTRVTTSDEDIGINAISRYSIVDTSLPFTINPNTGDIVISRPLNREDTDRYRIRVSAHDSGWTVSTDVTIFVTDINDNTPRFSRPSYYLDCPELPELGSRVTQVSATDPDEGSNGQVFYFIKSQSEYFRINATTGEIFNKQVLKYQNVSGFSNVNINRHSFIVTASDRGNPSLLSETTVTINTVDSNDNPPQFLQNKYFTPVTKNVKVGTKLIKVTAVDDKDFGLNSEVEYFVSDGNHLGKFKLDNDTGWISIASSLVSDLNQNFLIRVTAKDKGNPPLSSQAVVHITVTEENYHTPEFSQNHISATIPESHSIGSVVRTVSARDRDTAMNGLISYNIISGNEEGIFAINSSTGVVTLAKALDYEMSSKHEMTISATDGGWVARTGYCSLTVSVIDVNDNSPVFVPDEFFPTVMENAPSGTTVIHLNATDADSGANAVIAYTVQSSDSDLFVIDPNMGVITTQGFLDFETKQSYHLTVKAFNVPDEEKCSFATVDIQLKGTNEYVPRFVSKLYYFEVSEAASRGTAVGEVFASDRDMGADGEVHYLIFGNSRKKGFQINKMTGQIYVSGLLDREKEERVSLKVLAKNFGNIRGADIDEVTVNITVLDANDPPVFSLSTYRVQISEGVPIGTHVTFVSAFDSDSIPSWSRFSYFIGSGNENGAFSINPQTGQITVTSGLDRESLPVYNLTVLAVDSGTPSATGSASLVVTLEDINDNGPVLTVSEGEVLENKRPGTLVMTLQSTDPDLPPNQGPFNYYLLSTGPATNYFSLSTAGVLSTTREIDREQIADFYLSVVTRDSGAPQMSSTGTVHITVLDQNDNPSQSRTVEIFVNYYGNLFPGGTLGSVKPQDPDVLDSFHCSLTSGVTSLFSIPAGSCDLSSQPRSTDGTFDLTVVSSDGVHSTVTNNIRVFFAGFSNATIDNSILLRVGVPTVKDFLTNHYLHFLRIASSQLTGLGTAVQLYAAYEENNRTFLLAAVKRNNNQYVNPSGVATFFESIKEILLRQSGVKVESVDHDPCIHGPCQNGGSCLRRLAVGSALKIQESLPVIIVANEPLQPSQCKCVPGYAGSWCEVDIDECLPAPCHNGGTCHNLVGGFSCSCPEGFTGRACERDINECLPSPCKHGAVCQNFPGGFNCVCKTGYTGKMCESSVNYCECNPCFNGGSCQSGVESYYCHCPFGVFGKHCELNSYGFEELSYMEFPSLDPNNNYIYVKFATIKSHALLLYNYDNQTGERAEFLALEIAEERLRFSYNLGSGTYKLTTMKKVSDGQFHTVIARRAGMAASLTVDSCSENQEPGYCTVSNVAVSDDWTLDVQPNRVTVGGIRSLEPILQRRGHVESHDFVGCVMEFAVNGRPLEPSQALAAQGILDQCPRLEGTCARNPCQHGGTCVDFWSWQQCQCMEGLTGKYCEKSVTPDTALSLEGKGRLDYHMSQSEKREYLLTQSIRDTTLEPFGVNSLEVKFRTRSENGILIHIQESSNYTTVKIKNGKVHFTSDAGVAGKVERIIPEAYIADGHWHTFRISKNGSITVLSVDRIHNRDIVHPTQDFGGIEVLSMSLGGIPPNQAHRDTQTGFNGCIASVLYGGESLPFSGKHSLASISKTDPSVKIGCRGPNICASNPCWGDLLCINQWYAYKCVPPGDCASHPCQNGGSCEPGLLSGYTCSCPESHTGRTCETVVACLGVLCPQGKVCKAGSPGGHVCVQSQGPDEISLPLWAVPAIVGSCATALALLVLSLILCNQCRGKMPKNPKEEKKPKEKKKKGSENVAFDDPDNIPPYGDDLAVRKQPEGNPKPDIIERENPYLIFDETDIPHNSETIPSAPLASPEQEIEHYDIDNASSIAPSDADIIQHYKQFRSHTPKFSIQRHSPLGFARQSPMPLGASSLTYQPSSYGQGLRTSSLSHSACPTPNPLSRHSPAPFSKPSAFYRNSPARELHLPLRDGGTLEMHGDPCQPGMFNYATRLGRRSKSPQAMASHGSRPGSRLKQPIAQIPLESSPPVGLSIEEVERLNTPRPRNPSICSADHGRSSSEEDCRRPLSRTRNPADGIPAPESSSDSDSHDSFTCSEMEYDREKPVVYTSRMPKLSQVNESDADDEDNYGARLKPRRYHGRRAEGGPVGTPAAASGAADSTLKLGQQAGNFNWDNLLNWGPGFGHYVDVFKDLASLPEKAAGNEEGKSGAAKPAAKDGEAEQYV

SEQ ID NO: 5 - protein, 4983 aa, homo sapiens
  MISC_FEATURE: human Fat4 protein
MDLAPDRATGRPWLPLHTLSVSQLLRVFWLLSLLPGQAWVHGAEPRQVFQVLEEQPPGTLVGTIQTRPGFTYRLSESHALFAINSSTGALYTTSTIDRESLPSDVINLVVLSSAPTYPTEVRVLVRDLNDNAPVFPDPSIVVTFKEDSSSGRQVILDTATDSDIGSNGVDHRSYRIIRGNEAGRFRLDITLNPSGEGAFLHLVSKGGLDREVTPQYQLLVEVEDKGEPKRRGYLQVNVTVQDINDNPPVFGSSHYQAGVPEDAVVGSSVLQVAAADADEGTNADIRYRLQDEGTPFQMDPETGLITVREPLDFEARRQYSLTVQAMDRGVPSLTGRAEALIQLLDVNDNDPVVKFRYFPATSRYASVDENAQVGTVVALLTVTDADSPAANGNISVQILGGNEQRHFEVQSSKVPNLSLIKVASALDRERIPSYNLTVSVSDNYGAPPGAAVQARSSVASLVIFVNDINDHPPVFSQQVYRVNLSEEAPPGSYVSGISATDGDSGLNANLRYSIVSGNGLGWFHISEHSGLVTTGSSGGLDRELASQIVLNISARDQGVHPKVSYAQLVVTLLDVNDEKPVFSQPEGYDVSVVENAPTGTELLMLRATDGDLGDNGTVRFSLQEAETDRRSFRLDPVSGRLSTISSLDREEQAFYSLLVLATDLGSPPQSSMARINVSLLDINDNSPVFYPVQYFAHIKENEPGGSYITTVSATDPDLGTNGTVKYSISAGDRSRFQVNAQSGVISTRMALDREEKTAYQLQIVATDGGNLQSPNQAIVTITVLDTQDNPPVFSQVAYSFVVFENVALGYHVGSVSASTMDLNSNISYLITTGDQKGMFAINQVTGQLTTANVIDREEQSFYQLKVVASGGTVTGDTMVNITVKDLNDNSPHFLQAIESVNVVENWQAGHSIFQAKAVDPDEGVNGMVLYSLKQNPKNLFAINEKNGTISLLGPLDVHAGSYQIEILASDMGVPQLSSSVILTVYVHDVNDNSPVFDQLSYEVTLSESEPVNSRFFKVQASDKDSGANGEIAYTIAEGNTGDAFGIFPDGQLYIKSELDRELQDRYVLMVVASDRAVEPLSATVNVTVILEDVNDNRPLFNSTNYTFYFEEEQRAGSFVGKVSAVDKDFGPNGEVRYSFEMVQPDFELHAISGEITNTHQFDRESLMRRRGTAVFSFTVIATDQGIPQPLKDQATVHVYMKDINDNAPKFLKDFYQATISESAANLTQVLRVSASDVDEGNNGLIHYSIIKGNEERQFAIDSTSGQVTLIGKLDYEATPAYSLVIQAVDSGTIPLNSTCTLNIDILDENDNTPSFPKSTLFVDVLENMRIGELVSSVTATDSDSGDNADLYYSITGTNNHGTFSISPNTGSIFLAKKLDFETQSLYKLNITAKDQGRPPRSSTMSVVIHVRDFNDNPPSFPPGDIFKSIVENIPIGTSVISVTAHDPDADINGQLSYTIIQQMPRGNHFTIDEVKGTIYTNAEIDREFANLFELTVKANDQAVPIETRRYALKNVTILVTDLNDNVPMFISQNALAADPSAVIGSVLTTIMAADPDEGANGEIEYEIINGDTDTFIVDRYSGDLRVASALVPSQLIYNLIVSATDLGPERRKSTTELTIILQGLDGPVFTQPKYITILKEGEPIGTNVISIEAASPRGSEAPVEYYIVSVRCEEKTVGRLFTIGRHTGIIQTAAILDREQGACLYLVDVYAIEKSTAFPRTQRAEVEITLQDINDNPPVFPTDMLDLTVEENIGDGSKIMQLTAMDADEGANALVTYTIISGADDSFRIDPESGDLIATRRLDRERRSKYSLLVRADDGLQSSDMRINITVSDVNDHTPKFSRPVYSFDIPEDTIPGSLVAAILATDDDSGVNGEITYIVNEDDEDGIFFLNPITGVFNLTRLLDYEVQQYYILTVRAEDGGGQFTTIRVYFNILDVNDNPPIFSLNSYSTSLMENLPVGSTVLVFNVTDADDGINSQLTYSIASGDSLGQFTVDKNGVLKVLKALDRESQSFYNLVVQVHDLPQIPASRFTSTAQVSIILLDVNDNPPTFLSPKLTYIPENTPIDTVVFKAQATDPDSGPNSYIEYTLLNPLGNKFSIGTIDGEVRLTGELDREEVSNYTLTVVATDKGQPSLSSSTEVVVMVLDINDNNPIFAQALYKVEINENTLTGTDIIQVFAADGDEGTNGQVRYGIVNGNTNQEFRIDSVTGAITVAKPLDREKTPTYHLTVQATDRGSTPRTDTSTVSIVLLDINDFVPVFELSPYSVNVPENLGTLPRTILQVVARDDDRGSNSKLSYVLFGGNEDNAFTLSASGELGVTQSLDRETKERFVLMITATDSGSPALTGTGTINVIVDDVNDNVPTFASKAYFTTIPEDAPTGTDVLLVNASDADASKNAVISYRIIGGNSQFTINPSTGQIITSALLDRETKDNYTLVVVCSDAGSPEPLSSSTSVLVTVTDVNDNPPRFQHHPYVTHIPSPTLPGSFVFAVTVTDADIGPNSELHYSLSGRNSEKFHIDPLRGAIMAAGPLNGASEVTFSVHVKDGGSFPKTDSTTVTVRFVNKADFPKVRAKEQTFMFPENQPVSSLVTTITGSSLRGEPMSYYIASGNLGNTFQIDQLTGQVSISQPLDFEKIQKYVVWIEARDGGFPPFSSYEKLDITVLDVNDNAPIFKEDPFISEILENLSPRKILTVSAMDKDSGPNGQLDYEIVNGNMENSFSINHATGEIRSVRPLDREKVSHYVLTIKSSDKGSPSQSTSVKVMINILDENDNAPRFSQIFSAHVPENSPLGYTVTRVTTSDEDIGINAISRYSIMDASLPFTINPSTGDIVISRPLNREDTDRYRIRVSAHDSGWTVSTDVTIFVTDINDNAPRFSRTSYYLDCPELTEIGSKVTQVFATDPDEGSNGQVFYFIKSQSEYFRINATTGEIFNKQILKYQNVTGFSNVNINRHSFIVTSSDRGKPSLISETTVTINIVDSNDNAPQFLKSKYFTPVTKNVKVGTKLIRVTAIDDKDFGLNSEVEYFISNDNHLGKFKLDNDTGWISVASSLISDLNQNFFITVTAKDKGNPPLSSQATVHITVTEENYHTPEFSQSHMSATIPESHSIGSIVRTVSARDRDAAMNGLIKYSISSGNEEGIFAINSSTGILTLAKALDYELCQKHEMTISAIDGGWVARTGYCSVTVNVIDVNDNSPVFLSDDYFPTVLENAPSGTTVIHLNATDADSGTNAVIAYTVQSSDSDLFVIDPNTGVITTQGFLDFETKQSYHLTVKAFNVPDEERCSFATVNIQLKGTNEYVPRFVSKLYYFEISEAAPKGTIVGEVFASDRDLGTDGEVHYLIFGNSRKKGFQINKKTGQIYVSGILDREKEERVSLKVLAKNFGSIRGADIDEVTVNVTVLDANDPPIFTLNIYSVQISEGVPIGTHVTFVSAFDSDSIPSWSRFSYFIGSGNENGAFSINPQTGQITVTAELDRETLPIYNLSVLAVDSGTPSATGSASLLVTLEDINDNGPMLTVSEGEVMENKRPGTLVMTLQSTDPDLPPNQGPFTYYLLSTGPATSYFSLSTAGVLSTTREIDREQIADFYLSVVTKDSGVPQMSSTGTVHITVIDQNDNPSQSRTVEIFVNYYGNLFPGGILGSVKPQDPDVLDSFHCSLTSGVTSLFSIPGGTCDLNSQPRSTDGTFDLTVLSNDGVHSTVTSNIRVFFAGFSNATVDNSILLRLGVPTVKDFLTNHYLHFLRIASSQLTGLGTAVQLYSAYEENNRTFLLAAVKRNHNQYVNPSGVATFFESIKEILLRQSGVKVESVDHDSCVHGPCQNGGSCLRRLAVSSVLKSRESLPVIIVANEPLQPFLCKCLPGYAGSWCEIDIDECLPSPCHSGGTCHNLVGGFSCSCPDGFTGRACERDINECLQSPCKNGAICQNFPGSFNCVCKTGYTGKMCESSVNYCECNPCFNGGSCQSGVDSYYCHCPFGVFGKHCELNSYGFEELSYMEFPSLDPNNNYIYVKFATIKSHALLLYNYDNQTGDRAEFLALEIAEERLRFSYNLGSGTYKLTTMKKVSDGHFHTVIARRAGMAASLTVDSCSENQEPGYCTVSNVAVSDDWTLDVQPNRVTVGGIRSLEPILQRRGHVESHDFVGCIMEFAVNGRPLEPSQALAAQGILDQCPRLEGACTRSPCQHGGTCMDYWSWQQCHCKEGLTGKYCEKSVTPDTALSLEGKGRLDYHMSQNEKREYLLRQSLRGAMLEPFGVNSLEVKFRTRSENGVLIHIQESSNYTTVKIKNGKVYFTSDAGIAGKVERNIPEVYVADGHWHTFLIGKNGTATVLSVDRIYNRDIIHPTQDFGGLDVLTISLGGIPPNQAHRDAQTAGFDGCIASMWYGGESLPFSGKHSLASISKTDPSVKIGCRGPNICASNPCWGDLLCINQWYAYRCVPPGDCASHPCQNGGSCEPGLHSGFTCSCPDSHTGRTCEMVVACLGVLCPQGKVCKAGSPAGHVCVLSQGPEEISLPLWAVPAIVGSCATVLALLVLSLILCNQCRGKKAKNPKEEKKPKEKKKKGSENVAFDDPDNIPPYGDDMTVRKQPEGNPKPDIIERENPYLIYDETDIPHNSETIPSAPLASPEQEIEHYDIDNASSIAPSDADIIQHYKQFRSHTPKFSIQRHSPLGFARQSPMPLGASSLTYQPSYGQGLRTSSLSHSACPTPNPLSRHSPAPFSKSSTFYRNSPARELHLPIRDGNTLEMHGDTCQPGIFNYATRLGRRSKSPQAMASHGSRPGSRLKQPIGQIPLESSPPVGLSIEEVERLNTPRPRNPSICSADHGRSSSEEDCRRPLSRTRNPADGIPAPESSSDSDSHESFTCSEMEYDREKPMVYTSRMPKLSQVNESDADDEDNYGARLKPRRYHGRRAEGGPVGTQAAAPGTADNTLPMKLGQQAGTFNWDNLLNWGPGFGHYVDVFKDLASLPEKAAANEEGKAGTTKPVPKDGEAEQYV

SEQ ID NO: 6 - protein, 4979 aa, rattus norvegicus
  MISC_FEATURE: rat Fat 4 polypeptide
MNLAANRTPGRRRFPLPSPSLGQLLRVWGLLSLLPSPARVQAAEQRQVFQVMEEQPPGTLVGTIPTRPGFTYRLSESHALFAINSSTGALYTTATIDRESLPSDVVNLVVLSSSPTYPTEVRVLVRDLNDNAPVFPDPSIVVTFKEDSGSGRQVILDTATDSDIGSNGVDHRSYRIVRGNEAGRFRLDITLNPSGEGAFLHLVSKGGLDREVTPQYQLLVEVEDKGEPKRRGYLQVNVTVQDINDNPPVFGSSHYQAGVPEDAVVGSSVLQVAAADADEGTNADIRYRLQDEGTPFQMDPETGLITVREPLDFEARRQYSLTVQATDRGVPSLTGRAEALIQLLDVNDNDPVVKFRYFPATSRYASVDENAQVGTVVALLTVTDADSPAANGNISVQILGGNEQRHFEVQRSKVPNLSLIKVASALDRERIPSYNLTVSVSDNSGAPPTAEVQARSSVASLVIFVNDINDHPPVFEQQVYRVNLSEEVPPGSYVSGVSATDGDSGLNANLRYSIVSGNGLGWFHISEHSGLVTTSAAGGLDRELASQIVLNISARDQGVHPKVSYAQLVVSVLDVNDQKPVFSQPEGYEVSVVENAPTGTELLVLGATDGDLGDNGTVRFSLQEAESDQRLFRLDPVSGRLSTISSLDREEQAFYCLLILATDLGSPPQSSTVQVNVSLLDINDNSPVFYPVQYFAHIQENEPGGSYVTTVSATDPDMGPNGTVKYSISAGDRSRFQIHAKSGVISTKMALDREEKTAYQLQIVATDGGNLQSPSQAIVTVTVLDTQDNPPVFSQAAYSFVVFENVALGYHVGSVSATTMDLNANISYLITTGDQRGMFAMNPVTGQLTTASVIDREEQSFYQLRVVASGGAVTGDAVVNITVKDLNDNAPHFLQAVESIDAVENWQAGHSIFQAKAVDPDEGVNGRVLYSLKQNPKNLFTINEQNGNISLLGTLDVHAGSYQVEIVASDMGVPQLSSSIILTVYVHDVNDNPPVFDQISYEVTLSESEPVNSRFFKVQALDKDSGANGEIAYAITDGNSGEAFGIFPDGQLYIKSELDRELQDRYVLLVVASDRAVEPLSATVNVTVILEDVNDNRPLFNSTNYTFYFEEEQRAGSFVGKVSAVDKDFGPNGEVRYAFEVAQPDFELHAITGEITNTHKFDRESLMRRRGTAVFSFTVIATDQGLPQPLKDQATVHVYMKDINDNAPKFLKDFYQATVSETATNLTQVLRVSASDVDEGNNGLIHYSILKGNEERQFSIDRFSGQVTLVGKLDYEATPAYSLLIQAVDSGTVPLNSTCTLSIDILDENDNTPSFPKSTLFVDVLENMRIGELVSSVTATDSDSGVNADLHYTITGSNNHGTFSISPNTGSIFLAKKLDFETQSLYKLNITAKDQGRPPRSSTMSVVIHVRDFNDNPPSFPPGDIFKSIVENIPLGTSVISVTAHDPDADINGQLSYAIIQQMPRGNHFSIDEVKGTIYTSAEIDREFANLFELTVKANDQAVPIETRRYALKNVTILVTDLNDNVPIFISQNALAADPSAMIGSVLTTIMAADPDEGANGEVEYEIVNGDTDTFTVDRYSGDLRVASALVPSQLIYNLIVSATDLGPERRKSTTELTVILQGLDGPVFTQTKYITILKEGEPIGTNVISIEAASPRGSEAPVEYYIVSVRCEEKTVGRLFTIGRQTGVIQTAAILDREQGACLYLVDVYAIEKSSAFPRTQRAEVEITLQDINDNPPVFPTDTLDLTVEENIGDGSKIMQLTAMDADEGANALVTYALISGADDSFRIDPESGDLIATKRLDREHRSKYSLLVRADDGLQSSDMRINITISDVNDHTPRFSRPVYSFDIPEDTTPGSLVAAILATDDDSGVNGEISYVVEEDDGDGVFFLNPVTGVFNLTRALDYERQQYYILTVRAEDGGGQFTTIRAYFNILDINDNPPVFSLSSYSTSLMENLPLGSTVLVFNVTDADDGVNSQLSYSIASGDSLGQFAVDKHGVLKALKALDRESQSFYNLVIQVHDLPQPPASRFTSTAQVSIILLDVNDNPPMFLSPKLTYIPENTPIDTVVFKAQATDPDSGPNSYIEYTLLNPAGNKFSIGTIDGEVHLTGELDREEVSNYSLTVVATDKGQPPLSSSTEVVVMVLDINDNNPVFAQAMYRVQIKENTLTGTDIIQVSAADNDEGTNGQVRYGIVGGNTHQEFRIDSVTGAITVAKSLDRETIPAYILTVQATDRGSSPRTDSCTVAITLLDMNDFVPVFELSPYSVNVPENLGALPRTILQVVARDDDQGPNSQLSYVLLGGNEDNAFALTASGELRVTQSLDREARDHFVLVVTAADAGSPALTGTGTINIIVDDINDNVPTFASNMYFTAIPEDAPTGTDVLLVNASDADAATNAVISIIGGNSQFTINPSTGQIITSALLDRETKDNYTLVVVASDAGSPESLSSSTSVLVSVTDVNDNPPRFQHHPYVTHIPSPTPPGSFVFAVTVTDADIGPNSELHYSLSGRNSEKFHIDPLRGAIMAAGPLSGASEVTFSVHVKDGGSFPKTDSTTVTVRFANKADFPKVRAKEQTFMFPENQPVGTLVTTITGSSLRGETLSYYIASGNLGNAFQIDPLTGQVSISQPLDFEKIQKYIVWIEARDGGFPPFSSYEKLDITVLDINDNAPAFEEDPFVSEILENLSPRKILTVSATDKDSGPNGQLAYEIVNGNQESSFTINHATGEIRSIRPLDREKISYYELTVKSSDKGSPSQSTSVKVIISVLDENDNAPRFSQIFSAYVSENSPLGYTVTRVTTSDEDIGINAISRYSIADTALPFTINPNTGDIVISRPLNREDTDRYRIRVSAHDSGWTVSTDVTIFVTDINDNTPRFSRPSYYLDCPELTELGSRVTQVSATDPDEGSNGQVFYFIKSKSEYFRINATTGEIFNKQVLKYQNVSGFSNVNINRHSFIVTASDRGNPSLLSETTVTINTVDSNDNPPQFLKNKYFTPVTKNVKVGTKLIKVTAVDDKDFGLNSEVEYFVSNGNHLGKFKLDNDTGWISIASSLVSDLNQNFFIRVTAKDKGNPPLSSQVVIHITVTEENYHTPEFSQSHISATIPESHSIGSVVRTVSARDRDTAMNGLISYNISSGNEEGIFAINSSTGVVTLAKALDYELSSKHEMTISATDGGWVARTGYCSLTVSVIDVNDNSPVFVPDEFFPTVMENAPSGTTVIHLNATDADSGANAVIAYTVQSSDSDLFVIDPNTGVITTQGFLDFETKQSYHLTVKAFNVPDEEKCSFATVNIQLMGTNEYVPRFVSKLYYFEVSEAAPRGTAVGEVFASDRDMGADGEVHYLIFGNSRKKGFQIHKMTGQIYVSGLLDREKEERVSLKVLAKNFGNIRGADIDEVTVNITVLDANDPPVFSLNTYRVQISEGVPIGTHVTFVSAFDSDSIPSWSRFSYFIGSGNENGAFSINPQTGQITVTSGLDRESLPVYNLTVLAVDSGTPSATGSASLLVTLEDINDNGPVLTVSEGEVLENKRPGTLVMTLQSTDPDLPPNQGPFNYYLLSTGPATNYFSLNTAGVLSTTREIDREQIADFYLSVVTRDSGAPQMSSTGTVHITVLDQNDNPSQSRTVEIFVNYYGNLFPGGTLGSVKPQDPDVLDSFHCSLTSGVTSLFSIPAGSCDLSSQPRSTDGTFDLTVVSSDGVHSTVTNNIRVFFAGFSNATIDNSILLRVGVPTVKDFLTNHYLHFLRIASSQLTGLGTAVQLYAAYEENNRTFLLAAVKRNHNQYVNPSGVATFFESIKEILLRQSGVKVESVDHDPCIHAPCQNGGSCLRRLAVGSALKIQESLPVIIVANEPLQPFQCKCVPGYAGSWCEVDIDECLPAPCHNGGTCHNLVGGFSCSCSEGFTGRACERDINECLPSPCKHGAVCQNFPGGFNCVCKTGYTGKMCESSVNYCECNPCFNGGSCQSGVESYYCHCPFGVFGKHCELNSYGFEELSYMEFPSLDPNNNYIYVKFATIKSHALLLYNYDNQTGERAEFLALEIAEERLRFSYNLGSGTYKLTTMKKVSDGQFHTVIARRAGMAASLTVDSCSENQEPGYCTVSNVAVSDDWTLDVQPNRVTVGGIRSLEPILQRKGHVESHDFVGCIMEFAVNGRPLEPSQALAAQGILDQCPRLEGTCARNPCQHGGTCVDFWSWQQCQCMEGLTGKYCEKSVTPDTALSLEGKGRLDYHMSQSEKREYLLTQSIREATLEPFGVNSLEVKFRTRSENGILIHIQESSNYTTVKIKNGKVHFTSDAGVAGKVERIIPEAYVADGHWHTFRISKNGSSTVLSVDRTHSRDVVHPTQDFGGIEVLSMSLGGIPPNQAHRDTQTGFNGCIASVLYGGESLPFSGKHSLASISKTDPSVKIGCRGPNICASNPCWGDLLCINQWYAYKCVPPGDCASHPCQNGGSCEPGLLSGYTCSCPESHTGRTCETVVACLGVLCPQGKVCKAGSPGGHVCVQSQGPDEISLPLWAVPAIVGSCATALALLVLSLILCNQCRGKMPKNPKEEKKPKEKKKKGSENVAFDDPDNIPPYGDDLAVRKQPEGNPKPDIIERENPYLIFDETDIPHNSETIPSAPLASPEQEIEHYDIDNASSIAPSDADIIQHYKQFRSHTPKFSIQRHSPLGFARQSPMPLGASSLTYQPSSYGQALRTSSLSHSACPTPNPLSRHSPAPFSKPSAFYRNSPARELHLPLRDGGTLEMHGDPCQPGMFNYATRLGRRSKSPQAMASHGSRPGSRLKQPIAQIPLESSPPVGLSIEEVERLNTPRPRNPSICSADHGRSSSEEDCRRPLSRTRNPADGIPAPESSSDSDSHDSFTCSEMEYDREKPVVYTSRMPKLSQVNESDADDEDNYGARLKPRRYHGRRAEGGPVGTPAAASGAADSTLKLGQQAGNFNWDNLLNWGPGFGHYVDVFKDLASLPEKAAGNEEGKSGAAKPAAKDGEAEQYV

SEQ ID NO: 7 - DNA, 4200 bp, mus musculus
  misc_feature: mouse Yap1 cDNA
gtttgggcgtctggagccaaagtttctgtctcagttgggacgccgccgcggccgggggcaaagaaagggaggaaggaaggagctcgcggaggggaggggaggagaggggaggcggcctcgggcaaggagtgcagggcgatgcgggcgcgcgtcgcagccccccgaacctgagcgcagtgccccgagcgtcgaacgaggccgcagccatggagcccgcgcaacagccgccgccccagccggccccgcaaggccccgcgccgccgtccgtgtctccggccgggacccccgcggccccgcccgcacccccggccggccaccaggtcgtgcacgtccgcggggactcggagaccgacttggaggcgctcttcaatgccgtcatgaaccccaagacggccaacgtgcctcagaccgtgcccatgcggcttcgcaagctgcccgactccttcttcaagccgcctgagcccaagtcccactcgcgacaggccagtactgatgcaggtactgcgggagctctgactccacagcatgttcgagctcactcctctccagcctccctgcagctgggtgccgtttctcctgggacactcacagccagtggcgttgtctctggccctgccgctgcccctgcagctcagcatctccggcagtcctcctttgagatccctgatgatgtaccactgccagcaggctgggagatggccaagacatcttctggtcaaagatacttcttaaatcacaacgatcagacaacaacatggcaggacccccggaaggccatgctttcgcaactgaacgttcctgcgcctgccagcccagcggtgccccagacgctgatgaattctgcctcaggacctcttcctgatggatgggagcaagccatgactcaggatggagaagtttactacataaaccataagaacaagaccacatcctggctggacccaaggctggaccctcgttttgccatgaaccagaggatcactcagagtgctccagtgaagcagcccccacccttggctccccagagcccacagggaggcgtcctgggtggaggcagttccaaccagcagcagcaaatacagctgcagcagttacagatggagaaggagagactgcggttgaaacaacaggaattatttcggcaggcaatacggaatatcaatcccagcacagcaaatgctccaaaatgtcaggaattagctctgcgcagccagttgcctacactggagcaggatggagggactccgaatgcagtgtcttctcctgggatgtctcaggaattgagaacaatgacaaccaatagttccgatccctttcttaacagtggcacctatcactctcgagatgagagcacagacagcggcctcagcatgagcagctacagcatccctcggaccccagacgacttcctcaacagtgtggatgaaatggatacaggagacaccatcagccaaagcaccctgccgtcacagcagagccgcttccccgactacctggaagccctccctgggacaaatgtggaccttggcacactggaaggagatgcaatgaacatagaaggggaggagctgatgcccagtctgcaggaagcgctgagttccgaaatcttggacgtggagtctgtgttggctgccaccaagctagataaagaaagctttctcacgtggttatagagctgcagggagccactctgagtctgtgagggatccacagagcctaagatgtgcacgcctgaaattcagataagtcagtgggggttctctggctaacacagaaaacagatgaaccagtgtccatcgttgttccgcttttctctgcccgtcgctgctcttacgttggttgctgacctcttcacggccggctctaaagaacccgaaccgcagacagattcctttgttaactctgctatgataactacgttctctgggattgctgggggatggcctgctggataatggatgttctgccttttgtccggtggtcctttcaccatcactttaactgaacacacagactgggaactgaatgctctagaacattgttcaagaggtggtttcttcagctgccttgggtccaacaagccagaggcattgcgtctgatctcgtggaggacggaggggacccacgctgaagactggtgaactttccattcttctgttagcgatgccgttaggccatagtgacctgggtcttatcttagacgcttatgaggcatgagacagcttccatagaaatatattaattattaccacatactctagattaggtttgaatgaatattttctgtgggtgttttggttggtttttctctgcccccccccccttttttgtggttggtcttggtggaacgtaggcaaattaatgaattcgtttatagctgtagcttggggtgggcaataccattcttttggtgggaaatctgtatttcttggttttttaacatcctatttaaatcttaaatcttggttatctcctctctacatatatacacactcttattatgtctatggtagtgtgatagcagaatatatctttataaacatttctttcctttccagtttattttttaaatggtagccttgaacatatacatttagacacagtagtttatgtttcacaaatactttaaattctggttggagcaattgcaggtgtttgagggagggtgggcagtgttatagaaagggctctagtgggtaaagccaagaggcgcctcttgccctctgtgcactgtggtgacaacagaagccaacagtgagtggccttacttcccttagcctgatttcatgccatgtagtgtctgagctgtaagctttagagaggcaggcctgccttctgcctccacctcagctttgggtgtacactcagatactgagactgataatccacgaggtcaggttgagtgtcacctacaagatcatgcttaggaaacctcctcccgtgtctgctgagtggaccctggggtagtgtgcagaatgctgcgaggtcataggtaaagtgatcagactagcaagtgtggggtagttggactgagcagagctacaatgaagcatgattatggctttatttttgcaagattactagagtatgtgtctccaggagtaataatggtttccaaagagtattttttaaaggaacaaaataggcatgaattaactctgtgtataagctatgaagtaatagttggttatgaattatagtggcaccagctagcacctctgtcatttaagggtctttgagtgcttctagaagaatcccttattttcaagggttcataatgaacaacaattttcccccctggctaaagctgctactataaaatcctcagcttaggaagatagaaatcttttggccctttgatttagtatagcacaagaatgaagtagaaaagttttattcagtactttgagtgctcaaaaaatacaatcagtgtattatatgtgattcataggtcagtcatttgaggccattgactctaaggcttttaattagaaactagttgaagactcacccttgaattaagtctggggaaatggcctcccggaggatgcagtgactgtagaacagagatgaaattctactctagttgacaattgagcagatggatgatccacaacagtgcgtggggaatgggcagatgataggcgtgccgagcgcctatgattggccagcagctgcagaataaaggatggcgtcttctgtgggcagactagctgtagtgtggggagggggaggggaggtgggcatgagctgttgaaaggttaccgggctatgatatagaaaagcttttatgaacttgtttctcttcagtaaagacttatctcgctctcagtaaagaagttcctaagtttgcagctgcttaacctcccccacaaggtggatgtgccgtcttcctcctccctcagcatggtgcgccttgttatacctcaatgtccagcagcccctggtgcctgacagtgctctctgaccttatcaaaccctccatgctgactgtgggagatttaagcaccctgactctcactaaatttgaatcacggcctgaacgtagtctttcttgttgtgtccagcagtgtttaatgcttcctggctgtagtaggtagcatacaggtaagaacttaactggttattcttgaatttcttttacaataaaccaatttttataatcttaaaaaaaaaaaaaaaaaaaaaaaaaaaaaaaaa

SEQ ID NO: 8 - DNA, 5396 bp, homo sapiens
  misc_feature: human Yap 1 cDNA
gccgccgccagggaaaagaaagggaggaaggaaggaacaagaaaaggaaataaagagaaaggggaggcggggaaaggcaacgagctgtccggcctccgtcaagggagttggagggaaaaagttctcaggcgccgcaggtccgagtgcctcgcagcccctcccgaggcgcagccgccagaccagtggagccggggcgcagggcgggggcggaggcgccggggcgggggatgcggggccgcggcgcagccccccggccctgagagcgaggacagcgccgcccggcccgcagccgtcgccgcttctccacctcggcccgtggagccggggcgtccgggcgtagccctcgctcgcctgggtcagggggtgcgcgtcgggggaggcagaagccatggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatagagccctcaggcagactgaattctaaatctgtgaaggatctaaggagacacatgcaccggaaatttccataagccagttgcagttttcaggctaatacagaaaaagatgaacaaacgtccagcaagatactttaatcctctattttgctcttccttgtccattgctgctgttaatgtattgctgacctctttcacagttggctctaaagaatcaaaagaaaaaaactttttatttcttttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattagtcacataatgaccttgattttattttaggagcttataaggcatgagacaatttccatataaatatattaattattgccacatactctaatatagattttggtggataattttgtgggtgtgcattttgttctgttttgttgggttttttgttttttttgtttttggcagggtcggtgggggggttggttggttggttggttttgtcggaacctaggcaaatgaccatattagtgaatctgttaatagttgtagcttgggatggttattgtagttgttttggtaaaatcttcatttcctggttttttttaccaccttatttaaatctcgattatctgctctctcttttatatacatacacacacccaaacataacatttataatagtgtggtagtggaatgtatccttttttaggtttccctgctttccagttaatttttaaaatggtagcgctttgtatgcatttagaatacatgactagtagtttatatttcactggtagtttaaatctggttggggcagtctgcagatgtttgaagtagtttagtgttctagaaagagctattactgtggatagtgcctaggggagtgctccacgccctctgggcatacggtagatattatctgatgaattggaaaggagcaaaccagaaatggctttattttctcccttggactaatttttaagtctcgattggaattcagtgagtaggttcataatgtgcatgacagaaataagctttatagtggtttaccttcatttagctttggaagttttctttgccttagttttggaagtaaattctagtttgtagttctcatttgtaatgaacacattaacgactagattaaaatattgccttcaagattgttcttacttacaagacttgctcctacttctatgctgaaaattgaccctggatagaatactataaggttttgagttagctggaaaagtgatcagattaataaatgtatattggtagttgaatttagcaaagaaatagagataatcatgattatacctttatttttacaggaagagatgatgtaactagagtatgtgtctacaggagtaataatggtttccaaagagtattttttaaaggaacaaaacgagcatgaattaactcttcaatataagctatgaagtaatagttggttgtgaattaaagtggcaccagctagcacctctgtgttttaagggtctttcaatgtttctagaataagcccttattttcaagggttcataacaggcataaaatctcttctcctggcaaaagctgctatgaaaagcctcagcttgggaagatagatttttttccccccaattacaaaatctaagtattttggcccttcaatttggaggagggcaaaagttggaagtaagaagttttattttaagtactttcagtgctcaaaaaaatgcaatcactgtgttgtatataatagttcataggttgatcactcataataattgactctaaggcttttattaagaaaacagcagaaagattaaatcttgaattaagtctggggggaaatggccactgcagatggagttttagagtagtaatgaaattctacctagaatgcaaaattgggtatatgaattacatagcatgttgttgggattttttttaatgtgcagaagatcaaagctacttggaaggagtgcctataatttgccagtagccacagattaagattatatcttatatatcagcagattagctttagcttagggggagggtgggaaagtttggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgtattgggtagcattgggataagattttaactgggtattcttgaattgcttttacaataaaccaattttataatctttaaatttatcaactttttacatttgtgttattttcagtcagggcttcttagatctacttatggttgatggagcacattgatttggagtttcagatcttccaaagcactatttgttgtaataacttttctaaatgtagtgcctttaaaggaaaaatgaacacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtacttacataaattgttacattattttttcttatgtaatacctttttgtttgtttatgtggttcaaatatattctttccttaaactcttaaaaaaaaaa

SEQ ID NO: 9 - DNA, 1471 bp, rattus norvegicus
  misc_feature: rat Yap 1 cDNA
atggagcccgcgcaacagccgccgccccagccggccccgcaaggccccgcgccgccgtcggtgtctccggccgggacccccgcggccccgcccgcacccccggcgggtcaccaggtcgtgcacgtccgcggggactcggagaccgacctggaagcgcttttcaacgccgtcatgaaccccaagacggccaacgtgccacagaccgtgcccatgaggcttcgcaagctgcccgactccttcttcaagccgcctgagcccaagtcccactcgcgacaggccagtaccgatgcgggcactgctggagccctgactccccagcacgttcgagctcactcgtctccagcctccctgcagctgggggccgggacactcacggccagtggtgttgtctctggcccggccgccacccctgctgctcaacatctcagacagtcttcctttgagatccctgatgatgtaccattgccagcaggctgggagatggccaagacctcttctggtcagagatacttcttaaatcacaatgatcagacaacaacatggcaggacccccggaaggccatgctctcccaactgaacgttcctacatctgccagcccagcagtgccccagacgctgatgaactctgcctcagggcctcttcctgatggatgggagcaagccatgactcaggatggagaagtttactacataaaccataagaacaagaccacatcctggctggacccaaggcttgaccctcgttttgccatgaaccagaggatcactcagagtgctccagtgaagcagcccccacccttggctccccagagcccacagggaggcgtcctgggtggaggcagctcaaaccagcagcagcagatacagctgcagcagctacagatggagaaggagaggctgcgattgaaacagcaggagttatttcggcaggcaatacggaatatcaatcccagcacagcaaatgctccaaaatgtcagaccgtcagagcgggaattagctctccgcagccagttgccctcactggagcaggatggagggactcagaatgcagtgtcttctcccgggatgactcaggaattgaggacaatgacaaccaatagttcagatccctttcttaacagtggcacctatcactctagagatgagagcacagacagtggcctcagcatgagcagctacagcatccctcggaccccagacgacttcctgaacagcgtggatgaaatggacacaggagacaccatcagccagagcaccctgccgtcccagcagagccgcttccctgattacctggaggccctccctgggacaaatgtggaccttggcacactggaaggagatgcaatgaacatagaaggggaggagctgatgcccagccttcaggaagcgctgagttccgaaatcctggatgtggagtctgtgttggctgccaccaagttagataaagaaagctttctcacgtggttatag

SEQ ID NO: 10 - protein, 488 aa, mus musculus
  MISC_FEATURE: mouse Yap1 polypeptide
MEPAQQPPPQPAPQGPAPPSVSPAGTPAAPPAPPAGHQVVHVRGDSETDLEALFNAVMNPKTANVPQTVPMRLRKLPDSFFKPPEPKSHSRQASTDAGTAGALTPQHVRAHSSPASLQLGAVSPGTLTASGVVSGPAAAPAAQHLRQSSFEIPDDVPLPAGWEMAKTSSGQRYFLNHNDQTTTWQDPRKAMLSQLNVPAPASPAVPQTLMNSASGPLPDGWEQAMTQDGEVYYINHKNKTTSWLDPRLDPRFAMNQRITQSAPVKQPPPLAPQSPQGGVLGGGSSNQQQQIQLQQLQMEKERLRLKQQELFRQAIRNINPSTANAPKCQELALRSQLPTLEQDGGTPNAVSSPGMSQELRTMTTNSSDPFLNSGTYHSRDESTDSGLSMSSYSIPRTPDDFLNSVDEMDTGDTISQSTLPSQQSRFPDYLEALPGTNVDLGTLEGDAMNIEGEELMPSLQEALSSEILDVESVLAATKLDKESFLTWL

SEQ ID NO: 11 - protein, 504 aa, homo sapiens
  MISC_FEATURE: human Yap1 polypeptide
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 12 - protein, 366 aa, rattus norvegicus
  MISC_FEATURE: rat Yap1 polypeptide
MEPAQQPPPQPAPQGPAPPSVSPAGTPAAPPAPPAGHQVVHVRGDSETDLEALFNAVMNPKTANVPQTVPMRLRKLPDSFFKPPEPKSHSRQASTDAGTAGALTPQHVRAHSSPASLQLGAGTLTASGVVSGPAATPAAQHLRQSSFEIPDDVPLPAGWEMAKTSSGQRYFLNHNDQTTTWQDPRKAMLSQLNVPTSASPAVPQTLMNSASGPLPDGWEQAMTQDGEVYYINHKNKTTSWLDPRLDPRFAMNQRITQSAPVKQPPPLAPQSPQGGVLGGGSSNQQQQIQLQQLQMEKERLRLKQQELFRQAIRNINPSTANAPKCQTVRAGISSPQPVALTGAGWRDSECSVFSRDDSGIEDNDNQ

SEQ ID NO: 13 - DNA, 8951 bp, mus musculus
  misc_feature: mouse Amotl1 cDNA
accagcgaccctcctgggtagcacgctggcgactgggagcgcggggcgcccagggacccggcagcggggtggggtgggtgggactggagggggaacggggcatctctctgctgcccgagtgcagaggcacccttcgccgcgcctcccagccgccatccccctgccgccattcccctgccgccgcgttgcccaagccaggatcgtcccatgatcgccccatgcggagggcaaagtcgcgccggggaccttgcgagcctgtgcttagagctcctcctcccatctgctacagccccagcagtcctgtgcagattctagaagaccccgcctacttctacccagacttgcagctctattctgggaggcatgaagcatctactttgacagtggaggcaagtggaggcctaagggggaaaagcgtagaggaccctctctccagcttccactccccgaacttcctgaggacgcccgaggtggaaatgagaggttctgaggatgtagcctctggaagagtgttacagcggctgatccaggaacaactgcgctatggcaccccaactgagaacatgaacctgctggccattcagcaccaggccacagggagtgcagggccagcccacgccaccaccaacttttcttccacggaaaccctcactcaagaagatccacaaatggtctatcagtcggcccgccaagaaccgcagggtcaagagcatcagggagacaatacggtgatggagaagcaggtccggtccacacagcctcagcagaacaacgaggagctccccacgtatgaggaagccaaggcccagtcccagttcttcaggggacagcagcagcagcagcaacagcagcagcagcaacagcagcagcaacagcagcagggacaggggccccttagccacacttattacatggccggaggtaccagtcagaagtcccgcactgaggggaggcccacagtgaaccgggccaacagtggacaggcgcataaggatgaggccctgaaagaactaaagcagggccatgtccggtccctcagtgagaggatcatgcagctgtcactggagaggaatggggctaagcaacacctccccagctctggaaatggaaagagcttcaaagctggaggagagccatccccagctcagcctgtctgtaaagcactggaccctcgtggccctccacctgaataccccttcaagaccaagccaatgaaatccccagtcagcaagaaccaagatcacggtctttactacaatgaccagcaccctggggtactccatgagatggtcaaaccttacccagcacctcagcctgcgagaacagaagtggccgtcctgaggtatcagccacccccggagtatggcgtcaccagccggccctgccagctgccttttccatctacggtgcagcagcatagccccatgtcctctcagacctcctccatcggtggtactctgcactccgtctccctgcctcttccacttcccataagcctggcggcttcacagcccctaccagcctcccccaaccagcagcttggaccggatgcctttgcgattgtggagcgagcccagcaaatggtagagatcctgacagaggagaaccgtgtgcttcaccaggagcttcagggctgctatgacaacgctgacaagctccacaagtttgaaaaagagctgcagagtatttcggaggcctacgagagcctggtcaagtccaccaccaagcgtgagtctctggacaaggcaatgagaaccaagctcgaaggcgagataaggagacttcatgacttcaacagagatctccgagatcgactggagacagccaacaggcagctgtccagcagggaatacgatgggcatgaagacaaagctgcagagagccattacgtgtcccagaacaaagaattcttgaaggaaaaggaaaagttggaaatggagttggcagcagtgcgtacggcaagtgaggaccatcggaggcacatcgagatcctggaccaggctttgagcaatgcgcaggctagagtgatcaaactggaggaagagttacgagagaagcaagcctatgtggaaaaggtggagaagctgcagcaggccctgacccagctgcagtcagcgtgcgagaagcgagggcagatggaacgcaggttgcggacctggctggagagggagctagacgctctgaggacacagcagaaacatggcacaggccctccagtcagtctcccagaatgtaatgctcctgccctcatggagctggtgagggagaaggaggagcggatcctcgccttggaggccgacatgaccaagtgggagcagaagtacctggaagagagcaccatccggcactttgccatgagcgcagctgccgctgccacagccgagagggacaccaccatcagcaaccactcgaggaatggcagctatggggagagctccctggaggcccacatctggccagaggaagaagaagtggtgcaggccaacaggaggtgtcaggacatggagtacactattaaaaacctccatgccaaaatcatagagaaggatgccatgataaaggtcctgcagcagcgatcccgtaaggatgctgggaagacggactctgccagcctgaggcctgcccgctctgtcccatccatcgctgcggccactgggacacattctcgccagacttctcttaccagcagccagctgacagaagagaaaaaggaagagaagacgacctggaaagggagtataggattcctgctgggaaaggaacaccagggacaggcatctgcccctctgctgccgaccacacctgcctctgcattgtcccttccggcctctaccacatcggccagcagcacccacgccaagacgggcagcaaggacagcagcacacagaccgacaagagcacggagctcttctggcccagcatggcttccctccccagccgtggcaggctgagcacagccccttccaacagccccatcctaaagcatccagctgccaaaggagccgtggagaagcaggagaactctcctggtcatgggaaggcatcggagcacagaggccgagtcagcaacttgctgcacaagcctgagttcccagatggcgagatgatggaagtgctcatctagcgtcgtcctgtggcgcttgccagcatgctgacagatgagaacagaggctgaaagaagagctccagatgggcagttgcagtagcaactgcaaaaccaggacaggtgtggactcgtcacaatgttaccaaaacagctggctcactatctgcagagggagtgagagaggacgcatgtaggcttggaagcagagtagagaaggaggaggagtggaattttccattgtagagaaattgtatgtcttagggaggctccatgcttccccattcttggcttataaacagatgaagaagagctgggccttccttggaatccccttgttccctttgccttttagcaggtttggtttaagaagaatctgctagaaatcctgaaagcccatctcggcataatggtgtgtgtgtgtgtgtgtgtgtgtgtgtgtgtgtgtgtgtgtgtgtgtggtgtccttagcaaatacatccaggataatggggaatttcaagttttgtcaccttcttggctgtttcgtttgtggtttgtgatttcagagagtgtgaagctttgcttgtttggattggatggtattttctaagcaggatgtgagcaagatgcccttgccctcacccatcctgatttctttgagcttatctgtctcatctctacttactattttattgttgtatttttcctgtttaattagacatagcagagttccttatatatgggacttaggagactaccatccttgctgtggtgtccagatggacagagcctcgtgttggcaccccaactgctcttcccacctttgcttagcagaactgcagctgtcagccttattgtgagctagagatacctggctgaggagtccatgagttgctgacataacatcatgcctctgatgtgcatctctctcacgtccacactcttgctaggaggttgacaattacaccccatcgccctgtggggaagacttccttgtgaataggtgccacaggaacccatgatggcttacccagagctctctaggtagcatgggaggtctcagcaggcatccccagcctttcttgtatttgaagctctcaggtggtcttttgttgctctcccctgccccattctgccctggttgctgttgataagcacagtgccatccctgatcaattctttttccctgtgaatttgtttcataacccatcagactggtagacacacagcacaatcagtatcacagaaagggacacgacttacctgaagtgaggcagcaaagtcatggaaatgagtccctatgaacaagaggcaggacctgaggctgctgccacaagctctcatgttcattgctgtggaagctggcttcctggaaaccccagaagaagctgcccaggggagggtgaactatctccagatcagtaaggtttatttccaagtccccttcctcatcactcctttttggctcaagagaaaaatgaaaagtggtaattctgtgacgtcaagtccctcttaagtgcagcactgaaaatagcttggctcagagagctctgcttggatcatgtgtcctaggactcatctctaggatgagactgtggcctccccaggttgaggtggtacatgtattgtgagagacgtagcctggagaacagagaagaagagaggcaaggggaggagagagcagtggtgtttccctacaggaccctggaccatctgacaagacagaggaggtgggactagactacccacactccacttggcagtgggaaccaggctgggagcctcccaccccctccttaactcttctctcagatcttgcagcctgagtactgagcttgccctgtgccttgtggggaagggagtgagaaggcagcataagaggtcttgccagttgagacagaagcaggtaggtgcaggaactattttggatagggcatgttgttcagttgatggcaactaattctcttaagaacatattgtctcctattaatgttggggacaaacccagaaagggaagggtatagagatggggaattggtgagatcttccagccctgagaccatgtctgagccaccccaaacttaggctatctcccagcataggagtgagacactgcccagtttactcaaattctagtgtacgtgttccagacacattgcacacacagacagaaagtctcctgttccagccccctgcctccctgcgttgtcagtctgtttcagttgtctgaaaacaaaggggacacagaccagggctttaactgcaagtgaactgtggcttttgttcaagagtccgtgttgagagagatgggctatgggctttcagatagaggcccttcagtcttctgtccttgtaattctcttgaagggtgcatgatagaggagccccaggcactcgggagcctcgctgtgctctggggagttcatgggatctaaactctttcgacaccaaacaacagtactacataggtattttggtacaaactgaaagcactagaaaaagtgcctgttgaatggtcctcaaagcaccagagaaacgaggccaggttctccctcctggttgttcagatgcagaagctggacccaggaggcatacagcccatctccaagcttcacttaaagcaaagactcagaacaagaggctcagtcacaggctgcacggccttgaacatgcgcagcgctatgcctggtgctctgtggcccaatttgtgatacttgtgtgcacacagggaagctgtcacacctctcagggcctgaaaacaggactaaacccaagcctcatataactaaccacaccgccaccatccacaggcgtggatggaagtcttcatgttctgttataagcacaactacaaagcaaattaaaaaaataaataaataaaagtccttcctcatagtgcagaaggctcgttttcagatctcttggagcatttgatcatagaacttgagggtcttctcttgggataaagaaaggataaataaaggctgtggcacttagtggtggttggccagcatcctgctttgtccttgagtgagcagtgtggtgatgtctgaatacttccaggtgtcgtgtggaacctgagaagtattgagatcatgtctctgtccctgtggtttcagtaagtagattcttctatgtgtaagcactatttctctctggaggttcatagcactaagctgccaagaaaaacacgttcatttgcaaagaattaagagtgtgtgctaaacacttgacattgttctttgtttctcagaagctagtgaccagcaacgtggagtgttcagcaagtccccaccatgcctgcagggtggggctgctcccttccacacccagctttccttaggggtggagacagggaatggtgggacctgtgttgttccgtcccgcagaggtctctctttggagcccatgtataaggcttgacatttttctgtgagtagatttagaggcagttgttccaagtgaattctggcctcagtacaaagaatccagtggttctgacactcgggacaaaagctctggtatcccctgtagagaggctctgtcctgctttgctccagccactcacacatagttaataaagtggctgaagctcctcccttcccagagctgctgtgctggggacgctgtctgcagcccgcccgagtcccagagattggtgtgagcagtcagataaggctgatctgggagacggaggaggtggccatgacacaaggtgttgtcaggagaagatgattgtgacacaaggtgttgtcaggaggagatgactgtgacacaaggtgtcttcaagcagctcctttgtgagtaggctgcagagagccatcctgagtgggaggttgtgttgtttaaacacatggtgtcttctttgtctgcactggatggagcagggtttgtttctatgtgttctccaggtgagctcacaccctctttaagaaaaggggtcttgtggatgttggaagcaaggtctgtcaaacattcttaaatggtgatgctagagaaaagcgagccccctgtaacttccctaaaagccgactcagccactaagtatacatgttgactttcttctactttttttctagttaatcagtgtgatttaaatgtctttagctgatgtgaggactttttaaaaggcagtagtttgtggtgcagctccgtggaacaggttgtgtaataagaaacctcagaagccaggcttcaagggggccgggtgtgtgtgtgtgtgcggggtgcacccaggccagcctttcagtggattcctattctcatttagtaattgaatggtatattacattatatgtaagtgcatacatataaggtggaatttttctcaatataaaaaaagaaagaaagaaagaaagaaagaaagaaagaaagaaagagaaaaagaaagaaagaaatgtaagaaagagaaagaaatccttttgttttcctgtgagctcgctccttggggaggactacagggctccccatgtaaaatggctgttgactcctctagcacaatggcctccaaggtctggcctgctcctcggattccctttgaagtgagaggtcattgtcttttgagaatggatttaagttttgaaaatagcccacattgttccagcacaagggaacgaacatagggcaactagtcagaatcagaaccccagggccatcagatgacttggtgaaaaggcacttccaccaaagccaagacctgagtttgatccccaggaaccccatagtaggagagagccaactgcagcaagttgtcctctgactcagacacacacacacacacacacacacacacacacacacacgcgcgcgcacgcgcgcgcacatacatacacatgcacacacacacttacatgcacacacatgaacatgcacatgcacacacacacatgcatacatacatacacatgcacacacacatatatgcacacacatgaacatgcacacacacatacatgcacacacacatacatgcacacacatgcacacgcacacactcgtgcgcacacacagtttttagaagactacctaatgtgtaggaaaagggttattagtacctgcaacctctagggcctcgttttcaagcccgcttctctctccaggcttcatatgtgtgcaacccctgcatacactacagacctaagtacatataaatatgaacagccaataggtactctgtaaaatggatattcttaagtgcatgttattgttgtgtgtgccgtgactgaggtgtgtgttcgtcatgagactttgcttcttagctgaattaagactctgccctccctccgaggcaggaaataggctgcagcttctggaatcagcttctcacgttttcccgcctttgttgtgtttactcgtggctttgtctgagagtctgttctcacatacacacctgtaagtatttattacaaggtagaatggggacaggcagccacagtacttgtatttgaatgtttccagggagggacaatggagccccctcctcagtgaggtgctcacagaatctgtgtcaaattctgtgaaccagggccaacactggtaccagcttcaccctgacctggggccactgccctctctctccactgccttgtgtgtgtataggtgtggttgtgtgtgtgtgtctctgctgggggcatgtgccgcacttcccagcggactctggtatccatgtgtaacctttggcacctgtgttactggactgcattgacaggtggtgtacagatctgtacagatgggtctgtgtgaagagttggggagtcccccccacagtgaatatggatactgttggtcagccaaagattttcctattctttgctgcttaaactttgtgccttaatattgtaaataataaatgtttaaaacagcaaa

SEQ ID NO: 14 - DNA, 8999 bp, homo sapiens
  misc_feature: human Amotl1 cDNA
cggacggcggcgggagcgcgcgagaagctctaggacccagcagcggttgtcgggtttggggctggaggtgaagccctgtgtgaatggggttgattgtccggcgccacttccccgcgctgcccggcagccgtcttccccagccgagggactgaactagccatgatcgcctcatgtggagggcaaagttgcgccggggaacttgtgagcctgcggtgaaagggtccccttctgcttgttatagccccagtagtcctgtccaggttctagaagactccacctacttttccccagactttcagctctattctgggaggcatgaaacatctgctttgacggtggaggcaaccagtagcatcagggaaaaagttgttgaagatcctctttgtaacttccactccccaaacttcctgaggatctcagaggtggaaatgagaggttccgaggatgcggcagctggaacagtattgcagcggctgatccaggaacaactgcggtatggcaccccaaccgagaacatgaacttgctggccattcagcaccaggccacagggagtgcaggaccagcccatcctacaaacaacttttcttccacggaaaacctcactcaagaagacccacaaatggtctaccagtcagcacgccaagaaccgcagggtcaagaacaccaggtggacaatacggtgatggagaaacaggtccggtccacgcagcctcagcagaacaacgaggaactgcccacttacgaggaggccaaagcacagtcgcagttcttcagggggcagcagcagcagcaacagcagcagggggcggtgggccatggttactacatggcagggggcaccagtcagaagtcccgaactgaggggaggcccactgtgaaccgtgccaacagtggacaggcgcataaggacgaggcgctgaaggaactgaagcagggccacgtccgctcgctcagcgagagaatcatgcagctgtccctggagaggaatggggccaagcaacaccttcccggctcggggaatggaaagggcttcaaagtaggaggggggccctcccctgcccagcctgcaggtaaagtgctggaccctcggggtcctccacctgagtaccccttcaagaccaagcaaatgatgtccccagtcagcaagacccaggagcacggacttttttatggtgaccagcaccccgggatgctccacgagatggtcaagccctaccctgctcctcagcctgtgagaacagatgtggccgtcctgcggtaccagccaccccctgagtatggggtaacgagccgcccatgccaacttccgttcccatcaaccatgcagcagcacagccccatgtcctcccagacctcttccgccagcgggccactgcactctgtctccctgccgcttccactcccgatggccctgggtgctccacagcccccgcctgccgcctcccccagccagcagcttggtccagatgcctttgcgattgtggagcgagcccagcaaatggtggagatattaacagaggagaaccgggtgcttcaccaggaacttcagggttactacgacaatgccgacaagctccacaagtttgaaaaagaacttcagagaatttcggaagcctatgaaagtctggtcaagtctaccaccaagcgagaatcgctggacaaggccatgagaaacaaattggaaggcgagattagaagacttcatgatttcaacagagacctccgagatcgactagagactgctaacaggcaactatccagcagggaatacgaagggcatgaagacaaagctgcagaggggcattatgcttcccagaacaaagaattcttgaaggaaaaggagaaattagaaatggagttagcagcagtgcggactgcaagtgaggaccatcggagacacatcgagatcctggaccaggctttgagcaacgcccaggccagggtcatcaagctggaagaggagttacgagagaagcaagcatatgttgagaaagttgagaagctgcagcaggccctgacccagctgcagtctgcatgtgagaagcgagaacagatggagcggagactgcggacttggctggagagagagctggatgcactgagaacccagcagaaacatggaaatggccagccagccaacatgccggaatacaatgccccagccctcctggaacttgtgcgggagaaggaggagcggatcctggccctggaggccgacatgacaaagtgggagcagaagtacctggaggagagcaccatccgacactttgccatgaatgccgcagccactgcagcagctgagagggacaccacgatcatcaaccactcacggaatggcagctacggagagagctcgctggaggcccacatctggcaagaggaggaggaggtggtgcaggccaacagaaggtgtcaggacatggaatacactattaaaaatctccatgccaaaatcatagagaaagatgctatgattaaggtcctgcagcagcgatctcgtaaagatgccgggaagacagactcctccagcctacgtcctgcccgctccgttccatccatagcagcagctactgggacacactctcgccagacctctcttaccagcagccagctggctgaggaaaagaaggaagagaagacctggaaggggagcataggattgctgctggggaaggagcaccatgagcatgcctctgccccactgctgccacccccacccacctcagcactgtcctccatagcctccactacggcagccagcagtgcccacgccaagacaggcagcaaggacagcagcacacagactgacaagagtgccgagctcttctggcccagcatggcctcccttcccagccgcggccggctgagcacgacccctgctcacagccccgtcctgaaacacccagcggccaaagggaccgcagagaaactggagaactctcctggccatgggaagtcgcctgaccacagaggccgggtcagcagcttgctgcacaagcccgagttccctgatggagagatgatggaagtcctcatctaactgccatccctgtggaatttcagtacagaacactgacaaacaaggaaagcggcagagaaagaagaaagacctagaaggttgtagatgggaaatcaggaatgatttgaactgataaagatttcagactcataagaacacattttataaatgttaaacacaaaaactacatgactgaagatagaagagaatgcgatggattttattacacatggtggaagagagaagaggcgtgtaggtttgcaaacaaagttaagaaataggaaactgaatttttcattgtacagaaaatgtatctcttggggagggcctgtgtacccccattctctgattataaacagataaacccaaatgtggatcatccttggaatacccccattctccttgcctcttagcatgtttgatttaagaaaagcctccaggaaatcttgagagcctgtcttggcataatgcagtgtcacctgtgccaaccatggggtgtcccgtgagtgtgagtgtggcagtgagaagaagcatcgagggcgaggggaatatgaaagttggtcttcacctaattacctgtttggtttggattttcaagagatgattgggcttcttagcttttctgaattctgcagtattttctaaacaggatagattatgatctcggtccccatccatcctaacttctgagttcatctctgtctctgctttacagtctttaattttttgttgttccttcatttttttctttttaataagacatagcagaattccttaggtatgggatttatgagaccagtacctgaactgtcatcattcctactgtgtttgatagtgaccagtaggacaaagccacgtgccgacttcctggctgcttcacctttgcatagctgggttgcagctgcgaacctcattctgactgtgaactaagcaagctttgaggaggcaggagattccgtgggtccctgtgacatattgcattaagatcgtggcacttgcttttttgtctttcacatcggctgtcttgcaaggagcttgacatttccaaattccattgctctgtttggggaagacttaggacagcactccaggaaacagatgacaatttacagacagttgtctcagtggtttccagaatctcaccagcctcccgtatagcttcttgtattgaggctcatgagcgtattcacactctttcctcccccgcgcacgcctctgcttgctctcgatcagcatccagacctccgtggcaccaggttgcctctgttgtgaacttcttttgtaacctaccaggccagttgtatatacagtgttgtcaggtttttccgggtttcagttttgaggcagtttattcaaagtgagacagaagtgccacggaaaagaggcaggagtgtgttaggtggtgggtccattagacctctgctgtgacattgcatattcagctctgcagacactggcttcctttacaaatctaagagatgctggattagaaaagaggagaagctcatcgggccatcagagagatgctcctacagggtcctgatgtttttgtacctccaggtcagctggatcgctgttttcccagttccctttgctcatgcttacttagaggaagaaagaaaggggggtacctcttccaagtaccttctaaatgaaacactcaagagagtgctactcaggaaactttgcttggatcctaaaatggactggtcttgggtgtgtaaccccggtgaagttatagcctccccaaattgaggtgacagaaggaagacaagaggtgtaagctggagagggaagggaagaaatcagtggctttggccagcctctgtgccacccagtacgacagaggagtgggaactggccctctggggctctgcttggccataggcactgcacattgtgccacctgctcatcacctcctctagtctcacactgagcatcggagtacctgttgtgcagacaggaaaactgaggagctctgagaggctgagcatggagctcaccccatgccatagggtgtgggaagagggcacaggaggcctcatccatgggggaaagggttgaggatggacatgggtggggagagggcatagacatcccttcctaatctctgttcccaccacatttcataggagatgagttaggagatgacagctaactctcttaaggacattttgaccccagtttatgttggggatggaccagaaaggaaaatgtctagagataggaaggtaaatcaactctgccagcccctaccatcaggtctgggccaccccaaacttgggctgcctcccttagacataggttagagtgagagacccagctgtttctcccgaagcctcgtcttcatgccccagacacctgattgccccaatcaggtgtcagacttgcttgctcttcgaggtccccctgagctgccaagtgttttcattgtaaacagacacatcgccatgtttcagggcttcaacggcattgtattttggactttgaccgtattttttttctcttaacctactgagactcttccttaagaacaggatctatagctttaaaatgtcttcttctatgggaaatttcctgccaagccagggcactaaattctttcatcattagagctttcctgaagtccggccatatactgagtacctgctctgagttctgggcagcttacaatatcttaaacagattttcagaaaaataatgcaactatattttctagtgtcaactgtatgcaaacagtgtggtaaataccttttaattagtcctccacccacctggagtacagggggtgcgtttattattctgatttcactgatgaggaaatgggctcagaaaggttaagtcccttcctgaaatctcacagccaggagaagctaggatgatttaaaacaaagtttggagaggcactgggcatagaccctggctgtacagcctctaacatgcggcactgcatcttggcagtctctatgcctgtctgtgatgtgtgtgtgcacgtgtaactacacacacacacacacacacacacacacacacacacagctatcacacatctcagagcctaagaaataggactaagcccagaactcctagaatcacctataaatgctaggcatagatggaaattattgtgttccaccagaagcacagctccaaactatacctaaaaaatatttctgcacttcccagagacctggacttcaaactttcccagtggagcctgattatagaacttgagggtcctatctcaggatgaaggggagaggccctggcttcacgggaaggtattccagcattgttctgcttcacccttgactgcgttgtctggcagtttctgtgtgctgccaggatattatatggaactggagaagttggagtcaggtctctgaagctagagtttcactaattagatgcctctgtacatgagaactattactgtctgcaggtccatatagctaagctgccaggaaaaacacattatcttccaaaactttcagagcatgtgcagaaccctttcttagcgttttcttctcagcattttctctgcctcccagaggctggcagccagtgacactgcagagttcagcatgttctaaccatgcacgcagggcaggggctgccttggccctcctcaggctttcgttgggagagcaggcagtggtggagcccttctgggtgcagtcctctggggttgctctttggaactcatgtatgagtttgactccacaaggcttggcatggataccaaaacagttgcaacaaattagttctgaacctggaacagagaattcagtgcttctgttactcaggaaggaggtgttcagaatgccccgtgcagagcagccagtcattactcttgtttgttctcacctgggtgcgcaccctcatgatgcagtggctgtagcaccttcatgccaggtgctgagagaatgggaaattcttcctccccattgacctgagtcccagagacttagggacacagacttcaggtgaggctgcggacctcagaagcagtggataatagattggggcattaaaagcttttgaggcagggggctcatgttttgactgcagggagttatgctgagcaaagagatgtgtttttcaaaaccagggttcaaaaccagtgtccacgctggagtaagtggagcatgcttctctgtgttctctgaatgatcttgcactcctcttaagcaaaggagtaccatgaccatagtcagtgggatcccacaaatgttcttaaatgggtaaggctttaagtagccagagagtatccagcctaccattggcttctccacatcctaaaacctgagacagccttggtatatgctttataaatgtttcttttcttgttgtttaagtaattaaagtgtttaaaatgtcttcattagatgtgacgattgtttaatgagtttgcctctgacgtgtggctccatgggagataggcaaagtaattaagaagttaccagaaattggtcggctggggaaatgcaaaagttagcatttcagtagtgaatttctcctggaacaaatgagcaatttttcctctttctcttaagtagtatacccttttctcacttagtaatttaatggtatataaagacatgtgtataagtgagtgcatacatatgaggtatgactatagggttgtttgtgggaatttcttttcctaacatacagaagatcaaagtgttcatctcaccccgccctccttaaaaggtgtcttttgggagactatgtgctcattgactatagtgctgccaagtaaaaatatcttgggaactcttctactagaatggccttcagggcttggcatgttcctttggtttacccttagagatgagaaatcctcctcctttgaggatggatttaagttctggaaataatctcaagtgcttgatagcacagttggatgaaaaaagatggcaattaaggtaagttacaccatttttgtttctaaaaaaatccctaagaaatttcttggaatgagtctttggcctcagagcctctcaaagtgtccacttcaaggggggatcatcctcattagcacacagatttttaaaaatcaattctcttgccatgcctcctatgtgttcacatctctgcatacactacagatataagtgcataatcattcatataaacatctggtaggtattctgtaaaactgtgtttactttagtgcatgttattgtcatgttatgatgtgactggggtgtttctttgtcatgaaactttgcttcttcacagaattagaatactgctctctctatattgaactacatatacagcgttttcttgtatcagcccccaaagtctggatgcccggtgttgtgtttacatgtgattgtgcctaggagtctgttcacatagagacacctgtaagtatttattacaaaacggaatgtaagcaaatatatccacattggttttatttgaatcaaggtgtttttttgttttttgttttttttccttttgaggaggaacagggagcctcctcctccatgagcacttacagaattgtgtaaaattctgtgaaacagtggtaagcatgggcacccgatttcagctgtcctgctgccgctgccctccaacctgctctgtgtgtgtgtgtcgctgtgcttggtggcagtgtgccgtgctcgtgccggctcttcccagcagactggtatctggctgtaacgtttgacgtcttcatattgccagtctgtattgaggggtgatgtacatggccatacagccaaatgggtctgtgtaccagtgtggggattccaagaacactgcctgtcccccacagcaaatattgatgctgttggtcagccaaagattttcctctcctttgctgcttaaacttgtgccttaatattgtacataataaatggataaaatggcaaaatgactcttttctctcgcttgctcctttcttccttaaagaacttataaactgatgctcaataaatgtttcatttcattgtacccca

SEQ ID NO: 15 - DNA, 8127 bp, rattus norvegicus
  misc_feature: rat Amotl1 cDNA
  misc_feature (554)..(555): n is a, c, g, or t
gatctctgccccgacatgcaggcgtttgatgagagctgcatgcacgaggggctaggtggaagcatggatctcagtggaaactggactctggaggacggaagctggctgtcacccactggggatcgccacttccccaacggcagaggcgagagtgattttgtggacaccactcccgcatctttcagcccggaggagttagctcctccccccatctgctacagccccggcagtcctgcgcagattctagaagaccacgcctacttctacccagacttgcagctctattctgggaggcatgaagcacctactttgacagtggaggcaaacggaggcctgagggggaaaagcggtaagtcctcttgctgttggagtgcacgttgggagcccagagggtcggcgtttctcattttcacctcttctacattaagaaacccctgtgcccgtgactttagtttccaccactttcacacaatggtttcgatgacaatccggaaactctacaagggacaggggccgtcgagcacactttactacatgttccgcgaggtactggnnagtcagaagtcccgcaccgaggggaggcccaccgtgaaccgggccaacagcggacaagcacataaggatgaggccctaaaagaactaaagcagggccatgtccggtccctcagtgagaggatcatgcagctgtcgctggagaggaatggaactaagcaacacctccccagctcgggaaatggaaagagcttcaaagctggaggagagccatctcctgcccagcccgtctgtaaagcactggaccctcgtggccctccacctgaataccccttcaagaccaagccaatgaaatccccagtcagcaagaaccaagatcacggtctttactacagtgaccagcaccctggggtactccacgagatggtcaaaccttacccagcacctcagcctgcgagaacagaagtggccgtcctgaggtaccagccacccccagagtatggcgtcaccagtcgcccctgccagctgccttttccctccacggtgcagcagcatagccccatgtcctctcagacttcctccatcagtggtcctctgcactcggtctccctgcctcttccacttcccatgagcctggcggctccacagcccccaccggcctcccccagccagcagctcggaccagatgccttcgcgattgtggagagagcccaacaaatggtagagatcctcacagaggagaaccgtgtgcttcaccaggagcttcagggctgctatgacaacgccgacaagctccacaagtttgaaaaagagctgcagagtatctcagaggcctatgagagcctggtcaagtccaccaccaagcgtgagtccctggacaaggcaatgagaaacaagctcgaaggcgagataaggaggcttcatgacttcaacagagatctccgagatcgactggagacagccaacaggcagctgtccagcagggaatatgaagggcacgaagacaaagctgcagagagccatcacatgtcccagaataaagaattcttgaaggagaaggagaagttggaaatggagctggcagcagtgcggacggcgagcgaggaccaccggaggcatattgagatcctggaccaagctttgagcaatgcccaggccagagtgatcaagctggaggaagagttacgagagaagcaagcctatgtggagaaggtggagaagctgcagcaggccctgacccagctgcagtctgcgtgtgagaagcgagggcagatggagcgcaggttgcggacctggctggagagggagctggatgctctgaggacacagcagaaacatgggaatggccagcccgtcaatctcccggaatataatgctcctgccctcatggagcttgtgcgggagaaggaggagcggattctagccctggaggccgacatgaccaaatgggagcagaagtacctggaagagagcaccatccgacactttgccatgagcgccgccgccgctgccacggccgagagggacactaccatcagcaaccactcaaggaatggcagctatggtgagagctccctggaggcccacatctggccagaggaagaggaggtggtgcaggccaacaggcggtgtcaggacatggagtacactattaaaaacctccatgccaaaatcatagagaaggatgccatgataaaagtcctgcagcagcgatcccgtaaggatgctgggaagactgactcttccagtctgcggcctgcccgctctgtcccatccattgctgcggccaccgggacacattcccgtcagacttcactgaccagcggccagctggcagaagacaagaaggaagagaagacctggaaggggagtataggattcctgctggggaaggagcaccagggacaggcttctgcccctctgctgccagccacacctgcctctgcactgtccccgccgacctccaccacatcagccagcagcacccacgccaagacaggcagcaaggacagcagcacgcagaccgacaagatcactgagctcttctggcccagcatggcttccctcccgagccgcggcaggctcagcaccgccccttccaacagccccgtcctgaaacatccagctgccaaaggaaccgtggagaaacaggagaactctcctgggcacgggaagtcatcagaacacagaggccgagtcagcagcttgttgcagaagcctgagttcccggatggagagataatggaagtgctcatctaaccgtcgtcctgtggagtgtggtgccagcatgctgacagacgcactagaggctgcaagaagagagaagatccagatggtcagttgcagtagcaactgcaaaaccaggacagctgtggacaccgtgtcacagtgttaccaacgcagctggctcgtgtcacagtgttaccaacgcagctggctcgtgtcacagtgttaccaacgcagctggctcacttgtctgcagagggagtgaaagagaacgcatgcaggctgagaagcagagttgaggaggagtggaattttccattgtagagaaattgtatgttttggggaggttccatgcttccccgttcttggctgataaacagatgaagaaaagccagcctttccttggaatgaccttcttccctttgtctcttagcaggtttggtttaagaagaatcttggcataatgtagtgtgtgtgtgtgtgtgtgtgtgtgtgtgtgtgtgtgtgtgtgtgtgtgtgtgtgtccttagcagatacatccaggataatgggggattccacaggcaagttttgtcaccttcttggctgttttcatttgtggtttgtggtttcagagagtgtgaggctttgcttgtctggattgaatggtattttctgagcaggatgggagagagatgcccttggccctcatccgccctgacttctttgaacttatctgtcttgtctctacttattattttgttgtaatttttttctgtttaattagacatagcagagttccttacgtatgggacttaggagactaccatccatgctgtggtgtccaattggacagagcctcctgttgacaccccggctgcttccccacacctctgcttagccaaactgcagctgtcaatcttactgtgagctagggaaacttggatgaggagtccatgagtcgatagatagggtaacatcgtgccctgatgtgcctctcattcacatccgctctcttgcaaggaacttgacagtcacaccccaatgccctgtaggaaggcttccttgtgagcaggcaccacaggaaccccatgatggcttatgcagagctctctgggtggctgcgagatctcacaggcatcccccagccactttctgtatcggaagctctcagttctgttcattgctccctctggcctccttctgccctggctcctgttgatgagcacagtgccgtccctgatcagttctttcttcctgtgaatttgcttcgtagcccatcaggctggtagactggcagcacggtcagtgtcacccgagggacaggacttccctgaagtgaagcagcaatgtcatggaagcgaacccctgtgagcgagagacaggccccgaggctgctgccacaaggtctcatgttcattgctggggacactagcttcctacaaaccccaggagacgctgcccatagcacgggtggcccttcaggctcctgaactatctatccccagatcagtaggatttgttttccaagtccccttcctcattccttctttgctcaagagaaaaaagaaaagtggtaactctgtgactctaagtccctcttaaatgcagcgctgaatctagcttggctcggagatctctgacttgtccctagggtgagactgtggcctccccaggttgaggtggcacgtgtattgtgagagattagcctggagagcagagaagagggggaggggggatagggaggagagagcaatcgtgtctccctacaggaccctgggccatctgacaagacagaggaggtgggactggcttccccatgctgcacatggcagtgggaaccaggcttggagtcccccaccccctccttaacctcttctctcagaccttgtagccctagcttgccctgtgctctgcagggaaggggatgagaggacagcataagaagtcttgccagttgaggtggggctggaagatgaagcaggtgggtgtaggaatcattttggatagggcatgttgtttggttgacggcaactaattctcttaagagcatattgtctccagttaatgttggagacaacccagaaagggaagggtatagagatgggtaattaatgagatattccagccctggagcaatgtctgagccaccccaaacttaggctgtctcccagcataggagtggggcactgccccttactcaaattctagtctaagtgttccagacacattgtacacttggacaggaaatctcctgttccagcataccctctcccccataccctctcccccataccccctcccccataccttctcccccatactgttagtacgtttcagttgttcaaaagcaaagggggcgcagactaggctttaactgcatgtggagtgtagattttgcctaagaatccatgttgaagaagagagaggctatgtctttaaggtcgaggcccttcagtcttctgtccttgtaattctcttgaagcgtgagccccaggcactcagaagccatgttgtactccgggaagttcatgggacctgaactccttcaacaccgggaacaacagtactgcataggtactttggtactgactgtgcaagcactagaaaaagtacccgttgaatggccctcacagcatcagggaaacgaggccgagttcactctcctgattgctcagatgcagaagctggacccgtgaggtgtaccagcccacctccaagcttgacacagaggctagcacttaaagcaaagactcagaagaggagtctcagtcacaggctgcacagccttgaacatgatatctagaactatgcctgggtgatcggtggcccaacttgtgacacttgtgtgcacatagggaagctgacgctcctctcggggcctgaaaacaggactaaacccaaaccccatataactaaccataccactgccatccacaggcgtggatggaagtcttcacggtctgttacaagcacaagtacaaagccgataaaaataagtcaaagcccttcctcgtggtgcagagggctcctcctcagatctcttggagcctttgatcatagagcttgacggtctcctctcgggataaaggacaggtaaaggctgtggccttcagcggtgactgccccacaccctgctttccttaggggtggagacaaggaatggtggggccagtgttgttcagagagaagtctctctttggagcccatgtatggggcttgaccttttgctgtgagtgcatttagaggcaagtgaattctggcctcagtacagagaacccagcggtttcagcactcaagaaagaagccgcagtgtcccctacagagaggctctgtcctgcttggccccagccacctgcgccacacatttaataaagtggctgaaactcctcctttccaagagctagtgtgctgggtcctgtcctccccacagccttctgagtcccagagacttgtgtgagcagtcggataaggccaatctgggagacaggggatgaccatgacacaaggtgtttccaggcaggtcccttgtgcgtaggctgcagaaagctgtcctgactgggagcttgggttgtacgaacacatggtgccttctttgtctgtgctgaacgagtagggtgtgtttctctgtgttctctgagtaagatcacaccctctttaagcaaaggggtgttgtggatattagaagcaagattgataaatgttcttaaatggtggtgctagagaaaagtcagtccccgtaacttcctaaaagctgagtcagccagtctctaagtatgtacatgttggctttcttctattttttttctagttaatcagtgtgttttaaatgtttttagttgatgtgaggatttttttaaaggcatttgtgtgtggtgtggctccatgggagataggctgtgtaataagaaccctcagaagccagccagtcatccaaggggcgcccaagttcagtggatttcttcaggaattggagagcagtatttttccctcttttcagtagtatgcctgttctcatttagtaatttaatggtatattgcattatatgtgaatgcatacataaacggtggaatttctcttaatatccaaaaaagtccttctgttgccctctgagctctctccttggggaggactgcagggctcactgtgtaaaatggttgtggactcctctagcagaatggcttccaaggcctggcccattcctcagattccctttgaagtgagaggtcattgtcttttgagaatggatttaagttttggaaataccccacgctgttcacagtgcaagcgactgaacacagaacaattagtcagaatcagaatcccagggccatcaagatgactccacggaaaagggactgccaccaaccagagccaagacctgagtttggtccccaggaaccccatggcagcaggagagagtcagccgatgcaagtgtcacacacacacataaacacacacatacacatgcaacacatacacacaaacacatacatacacacacacaaacacatacacacatgcacacacacacacacacacacacacacacacacagagtttttggaagaatatctatgatgtaggaaaaggtttgttagtacctgcaacctctagggcctcatttccaagcctgctgcttctcttgccaggacttatacgtgtgcaacccctgcgtacactacagacctaagtacatactcgctaacatgaacacccagtaggtattctgtaaaacggatatacttaagtgcatgttattgtcgtgtgtgccgcgactgagctgtgtctttgtcatgaaactttgcttctttgccaaattaagactctgccctccctccgaggaaggaaatatacagcagtttctggaatcagcttctcaagttttcccgcctgtgttgtgtttacttgtggctttgcctgagagtctgtcctcacgtacacacctgtgagtatttattacaaggcagaacggggacaggcgtagccatagtacttgtgtttgaatgtttccagggagggacaatggagccccctcctcagtgaggtgctcacagaatctgtgtcaaattctgtgaacggggtccaacactagtaccagcttcaccccaaccctgagtgccctggagccactgccctctctactgccttgtgtgtgcatgggtgtggttgtgtgtgtgtctctgttggggcatgtgccgcatttcgcagtggactttggtatccacatgtaacctttggcatctgtgttcctggaccgcattgacagatctgtggtgtgcagatctgtgtagatgggtctgtgtgcaaagtggggagtcccctgaaagtgactatggatattgttggtcagccaaagactttcctattctttgctgcttaaactttgtgccttaatattgtaaatataaacggttaaaacaagcaa

SEQ ID NO: 16 - protein, 968 aa, mus musculus
  MISC_FEATURE: mouse Amotl1 polypeptide
MRRAKSRRGPCEPVLRAPPPICYSPSSPVQILEDPAYFYPDLQLYSGRHEASTLTVEASGGLRGKSVEDPLSSFHSPNFLRTPEVEMRGSEDVASGRVLQRLIQEQLRYGTPTENMNLLAIQHQATGSAGPAHATTNFSSTETLTQEDPQMVYQSARQEPQGQEHQGDNTVMEKQVRSTQPQQNNEELPTYEEAKAQSQFFRGQQQQQQQQQQQQQQQQQQGQGPLSHTYYMAGGTSQKSRTEGRPTVNRANSGQAHKDEALKELKQGHVRSLSERIMQLSLERNGAKQHLPSSGNGKSFKAGGEPSPAQPVCKALDPRGPPPEYPFKTKPMKSPVSKNQDHGLYYNDQHPGVLHEMVKPYPAPQPARTEVAVLRYQPPPEYGVTSRPCQLPFPSTVQQHSPMSSQTSSIGGTLHSVSLPLPLPISLAASQPLPASPNQQLGPDAFAIVERAQQMVEILTEENRVLHQELQGCYDNADKLHKFEKELQSISEAYESLVKSTTKRESLDKAMRTKLEGEIRRLHDFNRDLRDRLETANRQLSSREYDGHEDKAAESHYVSQNKEFLKEKEKLEMELAAVRTASEDHRRHIEILDQALSNAQARVIKLEEELREKQAYVEKVEKLQQALTQLQSACEKRGQMERRLRTWLERELDALRTQQKHGTGPPVSLPECNAPALMELVREKEERILALEADMTKWEQKYLEESTIRHFAMSAAAAATAERDTTISNHSRNGSYGESSLEAHIWPEEEEVVQANRRCQDMEYTIKNLHAKIIEKDAMIKVLQQRSRKDAGKTDSASLRPARSVPSIAAATGTHSRQTSLTSSQLTEEKKEEKTTWKGSIGFLLGKEHQGQASAPLLPTTPASALSLPASTTSASSTHAKTGSKDSSTQTDKSTELFWPSMASLPSRGRLSTAPSNSPILKHPAAKGAVEKQENSPGHGKASEHRGRVSNLLHKPEFPDGEMMEVLI

SEQ ID NO: 17 - protein, 956 aa, homo sapiens
  MISC_FEATURE: human Amotl1 polypeptide
MWRAKLRRGTCEPAVKGSPSACYSPSSPVQVLEDSTYFSPDFQLYSGRHETSALTVEATSSIREKVVEDPLCNFHSPNFLRISEVEMRGSEDAAAGTVLQRLIQEQLRYGTPTENMNLLAIQHQATGSAGPAHPTNNFSSTENLTQEDPQMVYQSARQEPQGQEHQVDNTVMEKQVRSTQPQQNNEELPTYEEAKAQSQFFRGQQQQQQQQGAVGHGYYMAGGTSQKSRTEGRPTVNRANSGQAHKDEALKELKQGHVRSLSERIMQLSLERNGAKQHLPGSGNGKGFKVGGGPSPAQPAGKVLDPRGPPPEYPFKTKQMMSPVSKTQEHGLFYGDQHPGMLHEMVKPYPAPQPVRTDVAVLRYQPPPEYGVTSRPCQLPFPSTMQQHSPMSSQTSSASGPLHSVSLPLPLPMALGAPQPPPAASPSQQLGPDAFAIVERAQQMVEILTEENRVLHQELQGYYDNADKLHKFEKELQRISEAYESLVKSTTKRESLDKAMRNKLEGEIRRLHDFNRDLRDRLETANRQLSSREYEGHEDKAAEGHYASQNKEFLKEKEKLEMELAAVRTASEDHRRHIEILDQALSNAQARVIKLEEELREKQAYVEKVEKLQQALTQLQSACEKREQMERRLRTWLERELDALRTQQKHGNGQPANMPEYNAPALLELVREKEERILALEADMTKWEQKYLEESTIRHFAMNAAATAAAERDTTIINHSRNGSYGESSLEAHIWQEEEEVVQANRRCQDMEYTIKNLHAKIIEKDAMIKVLQQRSRKDAGKTDSSSLRPARSVPSIAAATGTHSRQTSLTSSQLAEEKKEEKTWKGSIGLLLGKEHHEHASAPLLPPPPTSALSSIASTTAASSAHAKTGSKDSSTQTDKSAELFWPSMASLPSRGRLSTTPAHSPVLKHPAAKGTAEKLENSPGHGKSPDHRGRVSSLLHKPEFPDGEMMEVLI

SEQ ID NO: 18 - protein, 911 aa, rattus norvegicus
  MISC_FEATURE: rat Amotl1 polypeptide
  misc_feature (180)..(180): Xaa can be any naturally occurring amino acid
MQAFDESCMHEGLGGSMDLSGNWTLEDGSWLSPTGDRHFPNGRGESDFVDTTPASFSPEELAPPPICYSPGSPAQILEDHAYFYPDLQLYSGRHEAPTLTVEANGGLRGKSGKSSCCWSARWEPRGSAFLIFTSSTLRNPCARDFSFHHFHTMVSMTIRKLYKGQGPSSTLYYMFREVLXSQKSRTEGRPTVNRANSGQAHKDEALKELKQGHVRSLSERIMQLSLERNGTKQHLPSSGNGKSFKAGGEPSPAQPVCKALDPRGPPPEYPFKTKPMKSPVSKNQDHGLYYSDQHPGVLHEMVKPYPAPQPARTEVAVLRYQPPPEYGVTSRPCQLPFPSTVQQHSPMSSQTSSISGPLHSVSLPLPLPMSLAAPQPPPASPSQQLGPDAFAIVERAQQMVEILTEENRVLHQELQGCYDNADKLHKFEKELQSISEAYESLVKSTTKRESLDKAMRNKLEGEIRRLHDFNRDLRDRLETANRQLSSREYEGHEDKAAESHHMSQNKEFLKEKEKLEMELAAVRTASEDHRRHIEILDQALSNAQARVIKLEEELREKQAYVEKVEKLQQALTQLQSACEKRGQMERRLRTWLERELDALRTQQKHGNGQPVNLPEYNAPALMELVREKEERILALEADMTKWEQKYLEESTIRHFAMSAAAAATAERDTTISNHSRNGSYGESSLEAHIWPEEEEVVQANRRCQDMEYTIKNLHAKIIEKDAMIKVLQQRSRKDAGKTDSSSLRPARSVPSIAAATGTHSRQTSLTSGQLAEDKKEEKTWKGSIGFLLGKEHQGQASAPLLPATPASALSPPTSTTSASSTHAKTGSKDSSTQTDKITELFWPSMASLPSRGRLSTAPSNSPVLKHPAAKGTVEKQENSPGHGKSSEHRGRVSSLLQKPEFPDGEIMEVLI

SEQ ID NO: 19 - DNA, 15 bp, artificial
  siRNA against Fat4
ccgacccgagagatt

SEQ ID NO: 20 - DNA, 13 bp, artificial
  siRNA against Fat4
ccgccggaacgtt

SEQ ID NO: 21 - DNA, 13 bp, artificial
  siRNA against Amotl1
accaccaggggtt

SEQ ID NO: 22 - DNA, 16 bp, artificial
  siRNA against Amotl1
gagaccagaggccatt

SEQ ID NO: 23 - DNA, 17 bp, artificial
  siRNA against Amotl1
cacacagcccagaaatt

SEQ ID NO: 24 - DNA, 15 bp, artificial
  siRNA against  Yap1
gcagagaaccaaatt

SEQ ID NO: 25 - skipped

SEQ ID NO: 26 - RNA, 21 nt, artificial
  siRNA anti-Fat4
cggugcuagauaucaaugauu

SEQ ID NO: 27 - RNA, 21 nt, artificial
  siRNA anti-Yap1
ggucagagauacuucuuaauu